SEQUENCE LISTING (2 sequences)

SEQ ID NO: 1 - skipped

SEQ ID NO: 2 - RNA, 10 nt, synthetic construct
  source 1..10: mol_type = other RNA
aagcacaatc

(19) United States
(12) Patent Application Publication
(10) Pub. No.: US 2026/0176618 A1
(43) Pub. Date: Jun. 25, 2026

(54) CHEMICAL STABILITY OF MRNA

(71) Applicant: ModernaTX, Inc., Cambridge, MA (US)

(72) Inventors: Paul Yourik, Cambridge, MA (US); Caroline Köhrer, Cambridge, MA (US); David Reid, Cambridge, MA (US); Jamie Gilmore, Cambridge, MA (US)

(73) Assignee: ModernaTX, Inc., Cambridge, MA (US)

(21) Appl. No.: 19/126,788

(22) PCT Filed: Nov. 2, 2023
(86) PCT No.: PCT/US2023/078516

§ 371 Date: May 2, 2025

Related U.S. Application Data
(60) Provisional application No. 63/422,103, filed on Nov. 3, 2022.

(51) Int. Cl. C12N 15/11; A61K 9/00; A61K 9/51; A61K 31/7115; A61K 39/00; A61K 48/00; C12N 15/67; C12N 15/85; C12P 19/34

(52) U.S. Cl. CPC C12N 15/11; A61K 9/0019; A61K 9/5123; A61K 31/7115; A61K 39/00; A61K 48/0066; C12N 15/67; C12N 15/85; C12P 19/34; C12N 2830/50

(57) ABSTRACT

Aspects of the disclosure relate to mRNAs comprising a relatively low abundance of cytidine: adenosine (CA) dinucleotides that benefit from increased stability relative to mRNAs containing more CpA dinucleotides. The disclosure also relates to methods of modifying an mRNA sequence to improve stability. In some aspects, the disclosure relates to mRNAs comprising modified mRNA sequences with relatively reduced numbers of CpA dinucleotides, and compositions comprising mRNAs with relatively reduced numbers of CpA dinucleotides.

### RELATED APPLICATION

This application claims the benefit under 35 U.S.C. § 119 (e) of U.S. Provisional Application No. 63/422,103, filed Nov. 3, 2022, the contents of which are incorporated by reference herein in their entirety.

### BACKGROUND

Recently, messenger ribonucleic acid (mRNA)-based therapeutics have shown promise, e.g., as vaccines for infectious diseases. However, mRNAs are susceptible to cleavage through multiple pathways, such as hydrolysis of phosphodiester bonds. Unlike DNA and self-amplifying RNAs, which can generate additional mRNAs after introduction into cells, cleavage of administered mRNAs reduces the amount of protein that can be translated.

### SUMMARY

Described herein are RNAs (e.g., mRNAs) in which CpA dinucleotide content has been reduced, relative to a wild-type nucleic acid sequence, or minimized, to improve stability of the RNA. The disclosure is based, at least in part, on the discovery by the inventors that the phosphodiester bond between the cytidine and adenosine nucleotides of the CpA dinucleotide may be particularly susceptible to non-enzymatic cleavage (e.g., via spontaneous hydrolysis). These results are surprising, in part because previous reports in the literature suggested that the UA dinucleotide, rather than CA, is particularly susceptible to cleavage. See, e.g., Kierzek, Nucleic Acids Res. 1992. 20 (19): 5079-5084; and Kaukinen et al., Nucleic Acids Res. 2002. 30 (2): 468-474. Without wishing to be bound by theory, the inventors posit that reducing the abundance of CpA dinucleotides in an RNA sequence reduces the frequency of such spontaneous cleavage, thereby improving stability of the RNA (e.g., in stored RNA compositions). Such improved RNA stability provides multiple benefits in the production of RNA therapeutics and prophylactics. For example, the improved stability of RNAs in stored RNA compositions allows efficacy to be maintained for longer durations, thereby improving the efficiency of RNA manufacturing.

Reducing CpA dinucleotide content may be achieved by modifying one or more codons in the open reading frame (ORF) of the RNA without changing the amino acid sequence of an encoded protein. For example, one or more UCA codons encoding serine may be changed to UCU, UCC, or UCG, which still encode serine but do not contain a CpA dinucleotide. This same approach may be used to reduce or eliminate the presence of CpA dinucleotides in codons encoding proline, threonine, and/or arginine. The only amino acids that must be encoded by a codon containing a CpA dinucleotide are histidine (encoded by CAU and CAC) and glutamine (encoded by CAA and CAG), and so the theoretical minimum of CpA dinucleotides in an RNA sequence is limited only by the number of histidine and glutamine residues present in an encoded protein. As another example, methionine, isoleucine, threonine, lysine, and asparagine must be encoded by codons beginning with an adenosine (A) nucleotide, and so a preceding codon that ends in a cytidine (C) nucleotide will result in a CpA dinucleotide at the junction between the two codons. To eliminate such CpA dinucleotides, a first codon ending in a cytidine (C) nucleotide that immediately precedes a second codon encoding methionine, isoleucine, threonine, lysine, or asparagine may be changed to a codon that encodes the same amino acid as the first codon, but does not end in a C nucleotide. A first codon “immediately precedes” a second codon in a nucleic acid sequence if there are no intervening nucleotides between the last nucleotide of the first codon and the first nucleotide of the second codon (e.g., in the sequence GACAUG, the first codon (GAC) encoding aspartate immediately precedes the second codon (AUG) encoding methionine). The same approach may be applied to codons preceding serine- or arginine-encoding codons that begin with adenosine nucleotides. Alternatively, one or more serine-or arginine encoding codons that begin with adenosine nucleotides may be changed to codons that encode the same amino acid, but do not begin with adenosine nucleotides.

In addition or as an alternative to modifying the ORF, other untranslated regions (UTRs) of the RNA, such as the 5′ and 3′ UTRs, may be modified to reduce CpA dinucleotide abundance. In such UTRs, one or more nucleotides of a CpA dinucleotide may be mutated to eliminate CpA dinucleotides from the UTRs. Alternatively, a minimum number of CpA dinucleotides that are present in regulatory motifs may be maintained in a UTR. For example, a Kozak sequence that serves as the site of translation initiation may comprise one or more CpA dinucleotides, to allow efficient translation, while other CpA dinucleotides are eliminated to improve stability without reducing translation efficiency.

Codon and UTR modification to reduce CpA dinucleotide content may comprise specific substitutions maintain other features of an mRNA, such as nucleotide composition, codon optimality, and/or structure, within a desired range. For example, RNAs having higher % G/C contents (percentage of nucleotides in a sequence being guanosine or cytidine nucleotides) may be more stable than RNAs having lower % G/C contents. Without wishing to be bound by theory, the inventors posit that the formation of intramolecular secondary structures contributes to RNA thermodynamic stability, with G/C-rich RNAs forming more and stronger secondary structures. Thus, in modifying a codon to remove a CpA dinucleotide, a specific codon may be substituted to maintain or increase the % G/C content of the resulting RNA sequence. For example, a first codon ending in a cytidine nucleotide and preceding a second codon beginning with an adenosine nucleotide may be replaced by a codon ending in a guanosine nucleotide, if possible, to avoid reducing the % G/C content of the RNA sequence.

Accordingly, some aspects of the disclosure relate to a non-naturally occurring mRNA encoding a polypeptide, the mRNA comprising an open reading frame (ORF) encoding the polypeptide, wherein the ORF comprises a number of CpA dinucleotides that is greater than or equal to a theoretical minimum and less than or equal to 300% of the theoretical minimum.

Some aspects of the disclosure relate to a non-naturally occurring mRNA encoding a polypeptide, the mRNA comprising an open reading frame (ORF) encoding the polypeptide, wherein the ORF comprises a number of CpA dinucleotides that is: (i) greater than or equal to a theoretical minimum; and (ii) no more than 11 CpA dinucleotides per 100 nucleotides of the ORF greater than the theoretical minimum.

Some aspects of the disclosure relate to a non-naturally occurring mRNA encoding a polypeptide, the mRNA comprising an open reading frame (ORF) encoding the polypeptide, wherein the ORF comprises a CpA dinucleotide content of 6.5% or less.

Some aspects of the disclosure relate to an mRNA encoding a polypeptide, the mRNA comprising an open reading frame (ORF) encoding the polypeptide, wherein the mRNA has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%, wherein each of the uridine nucleotides of the ORF comprises a chemical modification, wherein: (a) fewer than 30% of amino acids that immediately precede an isoleucine residue in the polypeptide are encoded by codons in the ORF that end in cytidine nucleotides; (b) fewer than 30% of amino acids that immediately precede a methionine residue in the polypeptide are encoded by codons in the ORF that end in cytidine nucleotides; (c) fewer than 30% of amino acids that immediately precede a threonine residue in the polypeptide are encoded by codons in the ORF that end in cytidine nucleotides; (d) fewer than 30% of amino acids that immediately precede an asparagine residue in the polypeptide are encoded by codons in the ORF that end in cytidine nucleotides; (e) fewer than 30% of amino acids that immediately precede a lysine residue in the polypeptide are encoded by codons in the ORF that end in cytidine nucleotides; (f) fewer than 30% of amino acids that immediately precede a serine residue, wherein the serine residue is encoded by a codon in the ORF beginning with an adenosine nucleotide, are encoded by codons in the ORF that end in cytidine nucleotides; and/or (g) fewer than 30% of amino acids that immediately precede an arginine residue, wherein the arginine residue is encoded by a codon in the ORF beginning with an adenosine nucleotide, are encoded by codons in the ORF that end in cytidine nucleotides.

Some aspects of the disclosure relate to a lipid nanoparticle comprising an mRNA described herein, and an ionizable cationic lipid, a non-cationic lipid, a sterol, and a polyethylene glycol (PEG)-modified lipid.

Some aspects of the disclosure relate to a pharmaceutical composition comprising a lipid nanoparticle described herein, and a pharmaceutically acceptable excipient.

Some aspects of the disclosure relate to a method of producing a modified mRNA sequence comprising an ORF encoding a polypeptide, the method comprising modifying a reference mRNA sequence comprising a reference ORF to produce the modified mRNA sequence by: (a) replacing one or more codons in the reference ORF comprising a CpA dinucleotide with a codon that encodes the same amino acid but does not comprise a CpA dinucleotide; and/or (b) replacing one or more codons in the reference ORF that: (1) ends in a cytidine nucleotide; and (2) is immediately followed in the reference ORF by a codon that encodes an isoleucine, methionine, threonine, asparagine, or lysine, or a codon that encodes a serine or arginine and begins with an adenosine nucleotide, with a codon encoding the same amino acid as the replaced codon but does not end in a cytidine nucleotide.

### BRIEF DESCRIPTION OF THE DRAWINGS

FIG. 1 shows the results of sequencing mRNA fragments generated by spontaneous cleavage of a reference mRNA, as a frequency map of cleavage positions, used to determine the positions of spontaneous (non-enzymatic) cleavage. Sequencing reads were aligned to the full-length mRNA sequence, with the 3′ end of the read indicating the nucleotide in the mRNA sequence where cleavage occurred.

FIGS. 2A-2C show the effects of % G/C content and CpA dinucleotide abundance on mRNA structure and stability. FIGS. 2A and 2B show the kinetics of mRNA purity, as measured by FACE, during storage of unformulated mRNA at 40° C. (FIG. 2A) or 25° C. (FIG. 2B), for each of three mRNAs containing reduced CpA dinucleotide contents and for a control mRNA. FIG. 2B shows the kinetics of mRNA purity, as measured by reverse-phase ion pair (RPIP) chromatography, during storage of the same mRNAs formulated in lipid nanoparticles (LNPs) at 25° C.

FIGS. 3A-3C show the effects of CpA dinucleotide content in in vitro expression of a protein encoded by an mRNA. Lipid nanoparticles containing mRNAs were added to EXPI293 cells, and cells were analyzed by staining with an antibody specific to the protein, followed by flow cytometry to determine the percentage of cells expressing the encoded protein (Ag⁺ cells) (FIG. 3A), total fluorescence measured by the product of median fluorescence intensity and the frequency of protein-expressing cells (FIG. 3B), and normalized total fluorescence measured as the product of FIG. 3B divided by the product measured for mock-transfected cells (FIG. 3C).

FIG. 4 shows the effects of CpA dinucleotide abundance on immunogenicity of mRNAs comprised in lipid nanoparticles (LNP-mRNA compositions). Mice were administered two doses of the same LNP-mRNA composition on days 1 and 22, with sera collected on day 21, three weeks after administration of the first dose, and day 36, 14 days after administration of the second dose. All mRNAs tested encoded the same antigen with the same amino acid sequence, but individual mRNAs differed in CpA dinucleotide content.

### DETAILED DESCRIPTION

Aspects of the disclosure relate to non-naturally occurring (modified) mRNAs containing relatively reduced abundances of CpA dinucleotides, and methods of improving mRNA stability by reducing the number of CpA dinucleotides in the mRNA sequence. The disclosure is based, in part, on the discovery by the inventors that the CpA dinucleotide is the most susceptible to spontaneous cleavage in mRNAs containing 1-methylpseudouridine nucleotides in place of conventional uridine nucleotides. The compositions and methods described herein are useful, in some embodiments, for providing RNA therapeutics with improved stability, increased expression of encoded proteins, and/or improved efficacy.

Some aspects of the disclosure relate to a non-naturally occurring mRNA encoding a polypeptide, the mRNA comprising an open reading frame (ORF) encoding the polypeptide, wherein the ORF comprises a number of CpA dinucleotides that is greater than or equal to a theoretical minimum and less than or equal to 300% of the theoretical minimum.

Some aspects of the disclosure relate to a non-naturally occurring mRNA encoding a polypeptide, the mRNA comprising an open reading frame (ORF) encoding the polypeptide, wherein the ORF comprises a number of CpA dinucleotides that is: (i) greater than or equal to a theoretical minimum; and (ii) no more than 11 CpA dinucleotides per 100 nucleotides of the ORF greater than the theoretical minimum. In some embodiments, the number of CpA dinucleotides per 100 nucleotides of the ORF greater than the theoretical minimum is no more than 10, no more than 9, no more than 8, no more than 7, no more than 6, no more than 5, no more than 4, no more than 3, no more than 2, or no more than 1.

Some aspects of the disclosure relate to a non-naturally occurring mRNA encoding a polypeptide, the mRNA comprising an open reading frame (ORF) encoding the polypeptide, wherein the ORF comprises a CpA dinucleotide content of 6.5% or less. In some embodiments, the ORF comprises a CpA dinucleotide content of 6.0% or less, 5.5% or less, 5% or less, 4.5% or less, 4% or less, 3.5% or less, 3.0% or less, 2.5% or less, 2.0% or less, 1.5% or less, 1.0% or less, or 0.5% or less.

In some embodiments, (a) fewer than 30% of amino acids that immediately precede an isoleucine residue in the polypeptide are encoded by codons in the ORF that end in cytidine nucleotides; (b) fewer than 30% of amino acids that immediately precede a methionine residue in the polypeptide are encoded by codons in the ORF that end in cytidine nucleotides; (c) fewer than 30% of amino acids that immediately precede a threonine residue in the polypeptide are encoded by codons in the ORF that end in cytidine nucleotides; (d) fewer than 30% of amino acids that immediately precede an asparagine residue in the polypeptide are encoded by codons in the ORF that end in cytidine nucleotides; (e) fewer than 30% of amino acids that immediately precede a lysine residue in the polypeptide are encoded by codons in the ORF that end in cytidine nucleotides; (1) fewer than 30% of amino acids that immediately precede a serine residue, wherein the serine residue is encoded by a codon in the ORF beginning with an adenosine nucleotide, are encoded by codons in the ORF that end in cytidine nucleotides; and/or (g) fewer than 30% of amino acids that immediately precede an arginine residue, wherein the arginine residue is encoded by a codon in the ORF beginning with an adenosine nucleotide, are encoded by codons in the ORF that end in cytidine nucleotides. In some embodiments, the nucleotide sequence of the mRNA comprises a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%.

In some embodiments, one or more nucleotides of the mRNA comprises a chemically modified nucleotide. In some embodiments, each uridine nucleotide of the mRNA comprises a chemically modified nucleotide.

Some aspects of the disclosure relate to an mRNA encoding a polypeptide, the mRNA comprising an open reading frame (ORF) encoding the polypeptide, wherein the mRNA has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%, wherein each of the uridine nucleotides of the ORF comprises a chemical modification, wherein: (a) fewer than 30% of amino acids that immediately precede an isoleucine residue in the polypeptide are encoded by codons in the ORF that end in cytidine nucleotides; (b) fewer than 30% of amino acids that immediately precede a methionine residue in the polypeptide are encoded by codons in the ORF that end in cytidine nucleotides; (c) fewer than 30% of amino acids that immediately precede a threonine residue in the polypeptide are encoded by codons in the ORF that end in cytidine nucleotides; (d) fewer than 30% of amino acids that immediately precede an asparagine residue in the polypeptide are encoded by codons in the ORF that end in cytidine nucleotides; (e) fewer than 30% of amino acids that immediately precede a lysine residue in the polypeptide are encoded by codons in the ORF that end in cytidine nucleotides; (f) fewer than 30% of amino acids that immediately precede a serine residue, wherein the serine residue is encoded by a codon in the ORF beginning with an adenosine nucleotide, are encoded by codons in the ORF that end in cytidine nucleotides; and/or (g) fewer than 30% of amino acids that immediately precede an arginine residue, wherein the arginine residue is encoded by a codon in the ORF beginning with an adenosine nucleotide, are encoded by codons in the ORF that end in cytidine nucleotides.

In some embodiments, the chemically modified nucleotide comprise N1-methylpseudouridine.

In some embodiments, fewer than 15% of serine residues, fewer than 27% of proline residues, fewer than 28% of threonine residues, and fewer than 23% of alanine residues in the polypeptide are encoded by codons in the ORF comprising a CpA dinucleotide. In some embodiments, (a) no serine residue of the polypeptide is encoded by a codon in the ORF comprising a CpA dinucleotide; (b) no proline residue of the polypeptide is encoded by a codon in the ORF comprising a CpA dinucleotide; (c) no threonine residue of the polypeptide is encoded by a codon in the ORF comprising a CpA dinucleotide; and/or (d) no alanine residue of the polypeptide is encoded by a codon in the ORF comprising a CpA dinucleotide.

In some embodiments, (a) no amino acid that immediately precedes an isoleucine residue in the polypeptide is encoded by a codon in the ORF that ends in a cytidine nucleotide; (b) no amino acid that immediately precedes a methionine residue in the polypeptide is encoded by a codon in the ORF that ends in a cytidine nucleotide; (c) no amino acid that immediately precedes a threonine residue in the polypeptide is encoded by a codon in the ORF that ends in a cytidine nucleotide; (d) no amino acid that immediately precedes an asparagine residue in the polypeptide is encoded by a codon in the ORF that ends in a cytidine nucleotide; (e) no amino acid that immediately precedes a lysine residue in the polypeptide is encoded by a codon in the ORF that ends in a cytidine nucleotide; (f) no amino acid that immediately precedes a serine residue, wherein the serine residue is encoded by a codon in the ORF beginning with an adenosine nucleotide, is encoded by a codon in the ORF that ends in a cytidine nucleotide; and/or (g) no amino acid that immediately precedes an arginine residue, wherein the arginine residue is encoded by a codon in the ORF beginning with an adenosine nucleotide, is encoded by a codon in the ORF that ends in a cytidine nucleotide. In some embodiments, no amino acid that immediately precedes an isoleucine, methionine, threonine, asparagine, or lysine residue in the polypeptide is encoded by a codon that ends in a cytidine nucleotide. In some embodiments, no codon in the ORF beginning with an adenosine nucleotide is immediately preceded by a codon in the ORF that ends in a cytidine nucleotide.

In some embodiments, the ORF is codon-optimized for expression in a cell. In some embodiments, the cell is a mammalian cell. In some embodiments, the mRNA further comprises: (i) a 5′ untranslated region (UTR); and/or (ii) a 3′ UTR. In some embodiments, the 5′ UTR is a heterologous UTR and/or the 3′ UTR is a heterologous UTR. In some embodiments, the 5′ UTR comprises five or fewer, four or fewer, three or fewer, two or fewer, one or fewer, or zero CpA dinucleotides. In some embodiments, the 5′ UTR does not comprise a CpA dinucleotide. In some embodiments, the 3′ UTR comprises five or fewer, four or fewer, three or fewer, two or fewer, one or fewer, or zero CpA dinucleotides. In some embodiments, the 3′ UTR does not comprise a CpA dinucleotide. In some embodiments, the last nucleotide of the 5′ UTR is not a cytidine nucleotide.

In some embodiments, the 5′ UTR has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%. In some embodiments, the ORF has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%. In some embodiments, the 3′ UTR has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%. In some embodiments, the mRNA further comprises: (iii) a 5′ cap structure; and/or (iv) a poly-A tail. In some embodiments, the last nucleotide of the 3′ UTR is not a cytidine nucleotide. In some embodiments, the 5′ cap structure comprises 7 mG(5′)ppp(5) NImpNp.

In some embodiments, the level of expression in a mammalian cell of the encoded polypeptide from the mRNA is at least 50% of the level of expression of a reference mRNA comprising a reference open reading frame (rORF) encoding the polypeptide, wherein the TORF comprises a higher number of CpA dinucleotides than the ORF. In some embodiments, one or more CpA dinucleotides of the mRNA comprises a modified cytidine nucleotide and/or a modified adenosine nucleotide. In some embodiments, the number of CpA dinucleotides comprising an unmodified cytidine nucleotide and an unmodified adenosine nucleotide in the ORF is 100%, 95% or less, 90% or less, 80% or less, 70% or less, 60% or less, 50% or less, 40% or less, 30% or less, 20% or less, or 10% or less of the total number of histidine and glutamine residues in the polypeptide. In some embodiments, the polypeptide comprises 9-5,000, 20-4,000, 30-3,000, 40-2,000, or 50-1,500 amino acids. In some embodiments, the polypeptide is a vaccine antigen or a therapeutic protein.

In some embodiments, a coefficient of degradation at 25° C. of the mRNA is 90% or less, 80% or less, 70% or less, 60% or less, or 50% or less, relative to an mRNA comprising a wild-type ORF encoding the polypeptide. In some embodiments, a composition comprising a plurality of the mRNAs remains above 50% purity for at least 30 days, at least 60 days, at least 90 days, at least 120 days, at least 150 days, or at least 180 days longer in storage than a composition comprising a plurality of mRNAs comprising a wild-type ORF encoding the polypeptide. In some embodiments, storage of the mRNA is conducted at a temperature between about 2° C. to about 8° C. In some embodiments, the mRNA is stored in a buffer comprising 10-50 mM Tris and 5-10% sucrose, wherein the buffer has a pH of about 7.3 to about 7.6.

In some embodiments, the stability of the mRNA is increased relative to a reference mRNA having a higher number of CpA dinucleotides, the reference mRNA comprising a reference open reading frame (rORF) encoding the polypeptide, wherein the rORF has a higher number of CpA dinucleotides than the ORF.

Some aspects of the disclosure relate to a lipid nanoparticle comprising an mRNA described herein, and an ionizable cationic lipid, a non-cationic lipid, a sterol, and a polyethylene glycol (PEG)-modified lipid. In some embodiments, the lipid nanoparticle comprises 20-60% ionizable cationic lipid, and 5-25% non-cationic lipid, 25-55% cholesterol, and 0.5-15% polyethylene glycol (PEG)-modified lipid. In some embodiments, a coefficient of degradation at 25° C. of the mRNA in the lipid nanoparticle is 90% or less, 80% or less, 70% or less, 60% or less, or 50% or less, relative to an mRNA comprising a wild-type ORF encoding the polypeptide. In some embodiments, a composition comprising a plurality of the lipid nanoparticles remains above 50% purity for at least 30 days, at least 60 days, at least 90 days, at least 120 days, at least 150 days, or at least 180 days longer in storage than a composition comprising a plurality of the lipid nanoparticles and mRNAs comprising a wild-type ORF encoding the polypeptide. In some embodiments, storage of the lipid nanoparticle is conducted at a temperature between about 2° C. to about 8° C.

In some embodiments, the lipid nanoparticle further comprises a stabilizing compound of Formula (I):

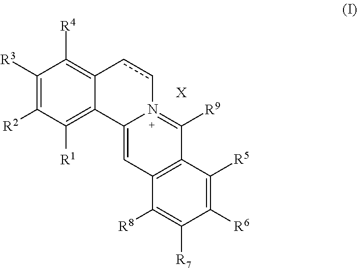

or a tautomer or solvate thereof, wherein:
[symbol] is a single bond or a double bond;
R¹ is H;
R² is OCH₃, or together with R³ is OCH₂O;
R³ is OCH₃, or together with R² is OCH₂O;
R⁴ is H;
R₅ is H or OCH₃;
R₆ is OCH₃;
R₇ is H or OCH₃;
R₈ is H;
R₉ is H or CH₃; and
X is a pharmaceutically acceptable anion.

In some embodiments, the stabilizing compound is wherein the compound is of:

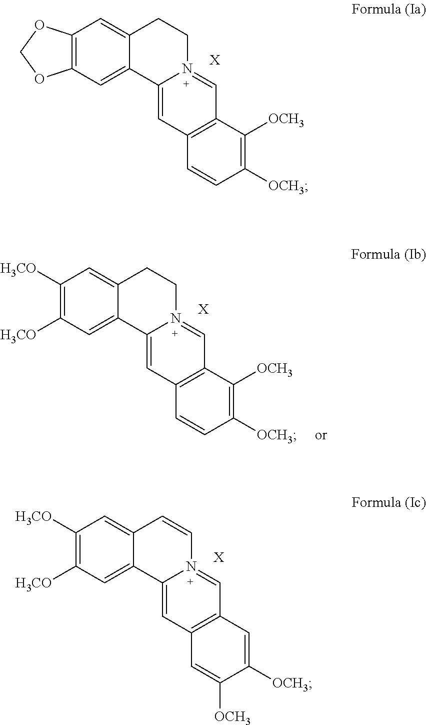

or a tautomer or solvate thereof.

In some embodiments, the lipid nanoparticle further comprises a stabilizing compound of Formula (II):

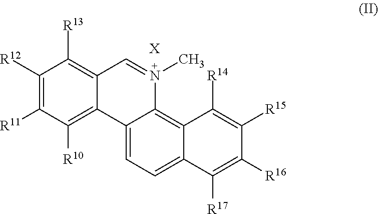

or a tautomer or solvate thereof, wherein:
R¹⁰ is H;
R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion.

Some aspects of the disclosure relate to a pharmaceutical composition comprising a lipid nanoparticle described herein, and a pharmaceutically acceptable excipient.

Some aspects of the disclosure relate to a method of producing a modified mRNA sequence comprising an ORF encoding a polypeptide, the method comprising modifying a reference mRNA sequence comprising a reference ORF to produce the modified mRNA sequence by: (a) replacing one or more codons in the reference ORF comprising a CpA dinucleotide with a codon that encodes the same amino acid but does not comprise a CpA dinucleotide; and/or (b) replacing one or more codons in the reference ORF that: (1) ends in a cytidine nucleotide; and (2) is immediately followed in the reference ORF by a codon that encodes an isoleucine, methionine, threonine, asparagine, or lysine, or a codon that encodes a serine or arginine and begins with an adenosine nucleotide, with a codon encoding the same amino acid as the replaced codon but does not end in a cytidine nucleotide.

In some embodiments, the reference mRNA sequence further comprises: (i) a reference 5′ untranslated region (UTR); and/or (ii) a reference 3′ UTR. In some embodiments, the reference 5′ UTR is a heterologous 5′ UTR and/or the reference 3′ UTR is a heterologous 3′ UTR. In some embodiments, the replacing comprises changing the last nucleotide of the reference 5′ UTR from a cytidine nucleotide to a non-cytidine nucleotide. In some embodiments, the reference mRNA sequence further comprises: (iii) a 5′ cap structure; and/or (iv) a poly-A region.

In some embodiments, the replacing comprises changing the last nucleotide of the reference 3′ UTR from a cytidine nucleotide to a non-cytidine nucleotide. In some embodiments, the method further comprises replacing one or more cytidine nucleotides in the reference mRNA sequence with guanosine nucleotides. In some embodiments, the method further comprises replacing one or more unmodified cytidine nucleotides in the reference mRNA sequence with modified cytidine nucleotides. In some embodiments, the method further comprises replacing one or more unmodified adenosine nucleotides in the reference mRNA sequence with modified adenosine nucleotides. In some embodiments, the method further comprises replacing one or more adenosine nucleotides in the reference mRNA sequence with uracil nucleotides. In some embodiments, the method further comprises replacing one or more adenosine nucleotides in the reference mRNA sequence, that are not immediately followed by a second adenosine nucleotide, with cytidine nucleotides. In some embodiments, the method further comprises replacing one or more adenosine nucleotides in the reference mRNA sequence with guanosine nucleotides.

In some embodiments, the ORF of the modified mRNA sequence comprises a number of CpA dinucleotides that is greater than or equal to the theoretical minimum and less than or equal to 300% of the theoretical minimum.

In some embodiments, the ORF of the modified mRNA sequences comprises a number of CpA dinucleotides that is: (i) greater than or equal to a theoretical minimum; and (ii) no more than 11 CpA dinucleotides per 100 nucleotides of the ORF greater than the theoretical minimum. In some embodiments, the number of CpA dinucleotides per 100 nucleotides of the ORF greater than the theoretical minimum is no more than 10, no more than 9, no more than 8, no more than 7, no more than 6, no more than 5, no more than 4, no more than 3, no more than 2, or no more than 1.

In some embodiments, the ORF of the modified mRNA sequence comprises a CpA dinucleotide content of 6.5% or less. In some embodiments, the ORF of the modified mRNA sequence comprises a CpA dinucleotide content of 6.0% or less, 5.5% or less, 5% or less, 4.5% or less, 4% or less, 3.5% or less, 3.0% or less, 2.5% or less, 2.0% or less, 1.5% or less, 1.0% or less, or 0.5% or less.

In some embodiments, in the modified mRNA sequence: (a) fewer than 30% of amino acids that immediately precede an isoleucine residue in the polypeptide are encoded by codons in the ORF that end in cytidine nucleotides; (b) fewer than 30% of amino acids that immediately precede a methionine residue in the polypeptide are encoded by codons in the ORF that end in cytidine nucleotides; (c) fewer than 30% of amino acids that immediately precede a threonine residue in the polypeptide are encoded by codons in the ORF that end in cytidine nucleotides; (d) fewer than 30% of amino acids that immediately precede an asparagine residue in the polypeptide are encoded by codons in the ORF that end in cytidine nucleotides; (c) fewer than 30% of amino acids that immediately precede a lysine residue in the polypeptide are encoded by codons in the ORF that end in cytidine nucleotides; (f) fewer than 30% of amino acids that immediately precede a serine residue, wherein the serine residue is encoded by a codon in the ORF beginning with an adenosine nucleotide, are encoded by codons in the ORF that end in cytidine nucleotides; and/or (g) fewer than 30% of amino acids that immediately precede an arginine residue, wherein the arginine residue is encoded by a codon in the ORF beginning with an adenosine nucleotide, are encoded by codons in the ORF that end in cytidine nucleotides.

In some embodiments, in the modified mRNA sequence, fewer than 15% of serine residues, fewer than 27% of proline residues, fewer than 28% of threonine residues, and fewer than 23% of alanine residues in the polypeptide are encoded by codons in the ORF that comprise a CpA dinucleotide. In some embodiments,, in the modified mRNA sequence: (a) no serine residue of the polypeptide is encoded by a codon in the ORF comprising a CpA dinucleotide; (b) no proline residue of the polypeptide is encoded by a codon in the ORF comprising a CpA dinucleotide; (c) no threonine residue of the polypeptide is encoded by a codon in the ORF comprising a CpA dinucleotide; and/or (d) no alanine residue of the polypeptide is encoded by a codon in the ORF comprising a CpA dinucleotide.

In some embodiments,, in the modified mRNA sequence: (a) no amino acid that immediately precedes an isoleucine residue in the polypeptide is encoded by a codon in the ORF that ends in a cytidine nucleotide; (b) no amino acid that immediately precedes a methionine residue in the polypeptide is encoded by a codon in the ORF that ends in a cytidine nucleotide; (c) no amino acid that immediately precedes a threonine residue in the polypeptide is encoded by a codon in the ORF that ends in a cytidine nucleotide; (d) no amino acid that immediately precedes an asparagine residue in the polypeptide is encoded by a codon in the ORF that ends in a cytidine nucleotide; (e) no amino acid that immediately precedes a lysine residue in the polypeptide is encoded by a codon in the ORF that ends in a cytidine nucleotide; (f) no amino acid that immediately precedes a serine residue, wherein the serine residue is encoded by a codon in the ORF beginning with an adenosine nucleotide, is encoded by a codon in the ORF that ends in a cytidine nucleotide; and/or (g) no amino acid that immediately precedes an arginine residue, wherein the arginine residue is encoded by a codon in the ORF beginning with an adenosine nucleotide, is encoded by a codon in the ORF that ends in a cytidine nucleotide. In some embodiments, in the modified mRNA sequence, no amino acid that immediately precedes an isoleucine, methionine, threonine, asparagine, lysine residue in the polypeptide is encoded by a codon that ends in a cytidine nucleotide. In some embodiments, in the modified mRNA sequence, no codon in the ORF beginning with an adenosine nucleotide is immediately preceded by a codon in the ORF that ends in a cytidine nucleotide.

In some embodiments, the modified mRNA sequence comprises a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%.

In some embodiments, one or more nucleotides of the modified mRNA sequence comprises a chemically modified nucleotide. In some embodiments, each of the uridine nucleotides of the modified mRNA sequence comprises a chemically modified nucleotide. In some embodiments, the chemically modified nucleotide comprises N1-methylpseudouridine.

In some embodiments, one or more CpA dinucleotides of the modified mRNA sequence comprises a modified cytidine nucleotide and/or a modified adenosine nucleotide. In some embodiments, the number of CpA dinucleotides comprising an unmodified cytidine nucleotide and an unmodified adenosine nucleotide in the ORF of the modified mRNA sequence is 100%, 95% or less, 90% or less, 80% or less, 70% or less, 60% or less, 50% or less, 40% or less, 30% or less, 20% or less, or 10% or less of the total number of histidine and glutamine residues in the polypeptide.

In some embodiments, the polypeptide comprises 9-5,000, 20-4,000, 30-3,000, 40-2,000, or 50-1,500 amino acids. In some embodiments, the polypeptide is a vaccine antigen or a therapeutic protein.

In some embodiments, the ORF of the modified mRNA sequence is codon-optimized for expression in a cell. In some embodiments, the cell is a mammalian cell. In some embodiments, the cell is a human cell.

In some embodiments, the method further comprises transcribing the modified mRNA sequence to produce a modified mRNA.

In some embodiments, a level of expression in a mammalian cell of the encoded polypeptide from the modified mRNA is at least 80% of a level of expression of the reference mRNA. In some embodiments, a coefficient of degradation at 25° C. of the modified mRNA is 90% or less, 80% or less, 70% or less, 60% or less, or 50% or less, relative to an mRNA comprising the reference ORF. In some embodiments, a composition comprising a plurality of the mRNAs is remains at least above 50% purity for at least 30 days, at least 60 days, at least 90 days, at least 120 days, at least 150 days, or at least 180 days longer in storage than a composition comprising a plurality of mRNAs comprising the reference ORF. In some embodiments, storage of the modified mRNA is conducted at a temperature between about 2° C. to about 8° C. In some embodiments, the modified mRNA has increased stability relative to a reference mRNA comprising the reference mRNA sequence.

CpA Dinucleotide Contents and mRNA Stability

Some aspects relate to mRNAs encoding polypeptides, the mRNA comprising an open reading frame (ORF) encoding the polypeptide, where the mRNA comprises a number of CpA dinucleotides content in the ORF that is at least equal to (i.e., equal to or greater than) a theoretical minimum number of CpA dinucleotides and at most (i.e., less than or equal to) 500% of the theoretical minimum. Other aspects relate to methods of modifying a reference mRNA sequence to produce a modified RNA sequence having fewer CpA dinucleotides than the reference mRNA sequence. As used herein, a “theoretical minimum” number of CpA dinucleotides refers to the number of histidine and glutamine residues present in a polypeptide encoded by an open reading frame. If a histidine or glutamine is present in an amino acid sequence, a codon beginning with CA is required to encode that amino acid, and so some CpA dinucleotides are required for a nucleic acid to encode a protein comprising histidine and/or glutamine residues. However, other amino acids that may be encoded by codons containing CpA dinucleotides (e.g., threonine, encoded by the codon ACA) may be also encoded by codons that do not contain a CpA dinucleotide (e.g., ACU, ACC, and ACG codons also encode threonine). Thus, portions of an mRNA sequence other than codons encoding histidine or glutamine may be mutated to reduce the number of CpA dinucleotides in an mRNA sequence to a level closer to the theoretical minimum. In some embodiments, the number of CpA dinucleotides in an ORF of a modified mRNA or modified sequence is 100%-400%, 100%-300%, 100%-200%, 100%-150%, or 100%-125% of the theoretical minimum. In some embodiments, the number of CpA dinucleotides is at most 400% of the theoretical minimum. In some embodiments, the number of CpA dinucleotides is at most 300% of the theoretical minimum. In some embodiments, the number of CpA dinucleotides is at most 250% of the theoretical minimum. In some embodiments, the number of CpA dinucleotides is at most 200% of the theoretical minimum. In some embodiments, the number of CpA dinucleotides is at most 150% of the theoretical minimum. In some embodiments, the number of CpA dinucleotides is at most 125% of the theoretical minimum.

References to the ORF of an mRNA, its length, the polypeptide it encodes, and codons within the ORF, are to be understood as referring to the longest ORF in the mRNA, not internal open reading frames in the same frame as the ORF, alternative reading frames, or sequences that may be translated due to initiation at a start codon that is downstream from the first occurrence of the sequence AUG in the mRNA.

Some aspects relate to mRNAs comprising an ORF encoding a polypeptide, with the ORF having a % CpA dinucleotide content of 6.5% or less. Some embodiments of such mRNAs contain ORFs with % CpA dinucleotide contents that are reduced, relative to a nucleic acid sequence encoding the same polypeptide (i.e., having the same amino acid sequence). The % CpA dinucleotide content (percentage CpA dinucleotide content) of a sequence can be determined by dividing the number of CpA dinucleotides in the sequence by the total number of dinucleotides in the sequence. Because consecutive dinucleotides in a nucleic acid sequence overlap (e.g., in an ORF beginning with the start codon AUG, the first dinucleotide is an AU dinucleotide, and the second dinucleotide is a UG dinucleotide), the number of dinucleotides in a sequence is one fewer than the number of nucleotides. For example, an ORF having 60 CpA dinucleotides and being 301 nucleotides in length has a % CpA dinucleotide content of 20%. In some embodiments, the ORF of an mRNA described herein has a % CpA dinucleotide content of 6.0% or less, 5.0% or less, 4.5% or less, 4.0% or less, 3.5% or less, 3.0% or less, 2.5% or less, 2.0% or less, 1.5% or less, 1.0% or less, or 0.5% or less. In some embodiments, the ORF has a % CpA dinucleotide content of 6.0% or less. In some embodiments, the ORF has a % CpA dinucleotide content of 5.5% or less. In some embodiments, the ORF has a % CpA dinucleotide content of 5.0% or less. In some embodiments, the ORF has a % CpA dinucleotide content of 4.5% or less. In some embodiments, the ORF has a % CpA dinucleotide content of 4.0% or less. In some embodiments, the ORF has a % CpA dinucleotide content of 3.5% or less. In some embodiments, the ORF has a % CpA dinucleotide content of 3.0% or less. In some embodiments, the ORF has a % CpA dinucleotide content of 2.5% or less. In some embodiments, the ORF has a % CpA dinucleotide content of 2.0% or less. In some embodiments, the ORF has a % CpA dinucleotide content of 1.5% or less. In some embodiments, the ORF has a % CpA dinucleotide content of 1.0% or less. In some embodiments, the ORF has a % CpA dinucleotide content of 0.5% or less.

In some embodiments of the modified mRNAs described herein or modified mRNA sequences produced by the methods described herein, an increased percentage of CpA dinucleotides in the ORF are comprised within codons encoding histidine or glutamine. A CpA dinucleotide is comprised within a codon if it forms either (i) the first and second nucleotides of a codon, or (ii) the second and third nucleotides of the codon, but not if it forms the third nucleotide of one codon and the first nucleotide of the second codon (i.e., the CpA dinucleotide bridges two codons). In some embodiments, at least 30%, at least 40%, at least 50%, at least 60%, at least 70%, at least 75%, at least 80%, at least 85%, at least 90%, at least 95%, at least 97%, at least 98%, at least 99%, or up to 100% of CpA dinucleotides in the ORF are comprised within codons encoding histidine or glutamine. In some embodiments, 30-100%, 30-80%, 30-50%, 40-100%, 40-90%, 40-80%, 40-60%, 50-100%, 50-90%, 50-80%, 50-70%, 50-60%, 60-100%, 60-90%, 60-80%, 60-70%, 70-100%, 70-90%, 70-80%, 80-100%, 80-90%, or 90-100% of CpA dinucleotides in the ORF are comprised within codons encoding histidine or glutamine. In some embodiments, at least 50% of CpA dinucleotides in the ORF are comprised within codons encoding histidine or glutamine. In some embodiments, at least 60% of CpA dinucleotides in the ORF are comprised within codons encoding histidine or glutamine. In some embodiments, at least 70% of CpA dinucleotides in the ORF are comprised within codons encoding histidine or glutamine. In some embodiments, at least 80% of CpA dinucleotides in the ORF are comprised within codons encoding histidine or glutamine. In some embodiments, at least 90% of CpA dinucleotides in the ORF are comprised within codons encoding histidine or glutamine. In some embodiments, at least 95% of CpA dinucleotides in the ORF are comprised within codons encoding histidine or glutamine. In some embodiments, 100% of CpA dinucleotides in the ORF are comprised within codons encoding histidine or glutamine.

In some embodiments of the modified mRNAs described herein or modified mRNA sequences produced by the methods described herein, the % CpA dinucleotide content in the ORF is reduced, relative to the % CpA dinucleotide content in a wild-type or reference ORF encoding the same polypeptide (e.g., having the same amino acid sequence). A “wild-type ORF,” as used herein, is the nucleotide sequence of a naturally occurring ORF that encodes the same polypeptide (having the same amino acid sequence) as the ORF of a modified mRNA or modified mRNA sequence, where the naturally occurring ORF is present on a naturally occurring mRNA. A “reference ORF,” as a starting sequence for modification to reduce % CpA dinucleotide content in a modified mRNA sequence, may be a wild-type ORF, or a non-naturally occurring ORF. In some embodiments, an ORF of a modified mRNA or modified mRNA sequence has a % CpA dinucleotide content that is 90% or less, 80% or less, 70% or less, 60% or less, 50% or less, 40% or less, or 30% or less of the % CpA dinucleotide content in a wild-type or reference ORF encoding the same polypeptide. In some embodiments, at least 20%, at least 30%, at least 40%, at least 50%, at least 60%, at least 70%, at least 80%, at least 90%, or at least 95% of CpA dinucleotides in the wild-type or reference ORF that are not comprised in a codon encoding histidine or glutamine are absent in a modified mRNA sequence encoding the polypeptide.

Some aspects relate to mRNAs comprising an ORF encoding a polypeptide, where the ORF comprises a number of CpA dinucleotides that is greater than or equal to a theoretical minimum, but the number of CpA dinucleotides above (greater than) the theoretical minimum is no more than 11 per every 100 nucleotides of the ORF. For example, an mRNA having a theoretical minimum of 20 CpA dinucleotides (due to encoding a polypeptide with a total of 20 histidine and/or glutamine residues), and encoding a protein that is 99 amino acids in length, thus having an ORF 300 nucleotides in length (including the STOP codon), could have 33 CpA dinucleotides above the minimum of 20 and still satisfy the requirement of having no more than 11 CpA dinucleotides per 100 nucleotides of the ORF above the theoretical minimum. In some embodiments, the number of CpA dinucleotides per 100 nucleotides of the ORF above the theoretical minimum is no more than 10. In some embodiments, the number of CpA dinucleotides per 100 nucleotides of the ORF above the theoretical minimum is no more than 9. In some embodiments, the number of CpA dinucleotides per 100 nucleotides of the ORF above the theoretical minimum is no more than 8. In some embodiments, the number of CpA dinucleotides per 100 nucleotides of the ORF above the theoretical minimum is no more than 7. In some embodiments, the number of CpA dinucleotides per 100 nucleotides of the ORF above the theoretical minimum is no more than 6. In some embodiments, the number of CpA dinucleotides per 100 nucleotides of the ORF above the theoretical minimum is no more than 5. In some embodiments, the number of CpA dinucleotides per 100 nucleotides of the ORF above the theoretical minimum is no more than 4. In some embodiments, the number of CpA dinucleotides per 100 nucleotides of the ORF above the theoretical minimum is no more than 3. In some embodiments, the number of CpA dinucleotides per 100 nucleotides of the ORF above the theoretical minimum is no more than 2. In some embodiments, the number of CpA dinucleotides per 100 nucleotides of the ORF above the theoretical minimum is no more than 1.

In some embodiments of the modified mRNAs described herein or modified mRNA sequences produced by methods described herein, the proportion of codons encoding a given amino acid is lower than the expected proportion based on codon usage frequencies in nature. For example, approximately 15% of serine residues in human proteins are encoded by codons having the RNA sequence UCA (DNA sequence TCA). Similarly, approximately 27% of proline residues are encoded CCA codons, approximately 28% of threonine residues are encoded by ACA codons, and approximately 23% of alanine residues are encoded by GCA codons. Thus, in some embodiments, (a) fewer than 15% of serine residues in an encoded polypeptide are encoded by codons comprising the sequence UCA; (b) fewer than 27% of proline residues are encoded by codons comprising the sequence CCA; (c) fewer than 28% of threonine residues are encoded by codons comprising the sequence ACA; and (d) fewer than 23% of alanine residues are encoded by codons comprising the sequence GCA. In some embodiments, fewer than 15%, fewer than 12%, fewer than 10%, fewer than 8%, fewer than 6%, fewer than 5%, fewer than 4%, fewer than 3%, fewer than 2%, or fewer than 1% of serine residues are encoded by UCA codons. In some embodiments, fewer than 27%, fewer than 25%, fewer than 20%, fewer than 15%, fewer than 12%, fewer than 10%, fewer than 8%, fewer than 6%, fewer than 5%, fewer than 4%, fewer than 3%, fewer than 2%, or fewer than 1% of proline residues are encoded by CCA codons. In some embodiments, fewer than 28%, fewer than 25%, fewer than 20%, fewer than 15%, fewer than 12%, fewer than 10%, fewer than 8%, fewer than 6%, fewer than 5%, fewer than 4%, fewer than 3%, fewer than 2%, or fewer than 1% of threonine residues are encoded by ACA codons. In some embodiments, fewer than 23%, fewer than 20%, fewer than 15%, fewer than 12%, fewer than 10%, fewer than 8%, fewer than 6%, fewer than 5%, fewer than 4%, fewer than 3%, fewer than 2%, or fewer than 1% of alanine residues are encoded by GCA codons. In some embodiments, fewer than 2% of serine residues are encoded by codons comprising the sequence UCA. In some embodiments, fewer than 12% of proline residues are encoded by codons comprising the sequence CCA. In some embodiments, fewer than 3% of threonine residues are encoded by codons comprising the sequence ACA. In some embodiments, fewer than 5% of alanine residues are encoded by codons comprising the sequence GCA. In some embodiments, no serine residue is encoded by a codon comprising the RNA sequence UCA. In some embodiments, no proline residue is encoded by a codon comprising the sequence CCA. In some embodiments, no threonine residue is encoded by a codon comprising the sequence ACA. In some embodiments, no alanine residue is encoded by a codon comprising the sequence GCA. In some embodiments, each serine, proline, threonine, and alanine residue is encoded by a codon that does not comprise a CpA dinucleotide. In some embodiments, none of the serine, proline, threonine, and alanine residues is encoded by a codon comprising a CpA dinucleotide. Replacement of codons encoding serine, proline, threonine, and/or alanine is contemplated because such codons may contain CpA dinucleotides in humans, but similar approaches are contemplated for reducing numbers of CpA dinucleotidesin mRNAs suitable for introduction into cells with different genetic codes in which other amino acids may be encoded by codons containing CpA dinucleotides.

In some embodiments of the modified mRNAs described herein or modified mRNA sequences produced by methods described herein, the proportion of codons immediately preceding a codon encoding a given amino acid is lower than the expected proportion based on codon usage frequencies in nature. For example, approximately 30% of codons in human open reading frames end in cytidine nucleotides. When such a codon ending in a cytidine (C) nucleotide is immediately followed by a codon encoding isoleucine, methionine, threonine, asparagine, or lysine, which must begin with an adenosine (A) nucleotide, a CpA dinucleotide is formed at the junction between the first (5′) and second (3′) codon. While codons encoding isoleucine, methionine, threonine, asparagine, and lysine cannot be mutated to begin with a different nucleotide without changing the encoded amino acid, an upstream codon may be substituted with a codon that does not end in a cytidine nucleotide, to reduce the abundance of CpA dinucleotides formed at the junction between two codons. Similarly, serine may be encoded by codons comprising the sequence AGU or AGC, and arginine may be encoded by codons comprising the sequence AGA or AGG. Therefore, substituting the codons immediately preceding such serine-encoding AGU and AGC codons, and/or such arginine-encoding AGA and AGG codons, may also reduce the abundance of such CpA dinucleotides at the junctions between two codons. Unlike isoleucine, methionine, threonine, asparagine, and lysine, however, serine and arginine may also be encoded by codons that do not begin with adenosine nucleotides. Instead, serine may be encoded by codons beginning with UC and ending with a guanosine, uridine, or cytidine nucleotide, and arginine may be encoded by codons beginning with CG and ending with any third nucleotide. Thus, codons encoding serine or arginine, and beginning with adenosine nucleotides, may be substituted with alternative codons that encode the same amino acid but do not begin with an adenosine nucleotide. Replacement of codons immediately preceding codons encoding isoleucine, methionine, asparagine, lysine, serine, or arginine, is specifically contemplated because all codons encoding isoleucine, methionine, asparagine, and lysine, and certain codons encoding serine and arginine, begin with adenosine nucleosides in humans, but similar approaches are contemplated for reducing numbers of CpA dinucleotides in mRNAs suitable for introduction into cells with different genetic codes in which other amino acids are encoded by codons beginning with adenosine residues.

In some embodiments of the modified mRNAs described herein or modified mRNA sequences produced by methods described herein, fewer than 30% of codons beginning with an adenosine nucleotide are immediately preceded by a codon ending in a cytidine nucleotide. In some embodiments, 25% or fewer of codons beginning with an adenosine nucleotide are immediately preceded by a codon ending in a cytidine nucleotide. In some embodiments, 20% or fewer of codons beginning with an adenosine nucleotide are immediately preceded by a codon ending in a cytidine nucleotide. In some embodiments, 15% or fewer of codons beginning with an adenosine nucleotide are immediately preceded by a codon ending in a cytidine nucleotide. In some embodiments, 12% or fewer of codons beginning with an adenosine nucleotide are immediately preceded by a codon ending in a cytidine nucleotide. In some embodiments, 10% or fewer of codons beginning with an adenosine nucleotide are immediately preceded by a codon ending in a cytidine nucleotide. In some embodiments, 8% or fewer of codons beginning with an adenosine nucleotide are immediately preceded by a codon ending in a cytidine nucleotide. In some embodiments, 6% or fewer of codons beginning with an adenosine nucleotide are immediately preceded by a codon ending in a cytidine nucleotide. In some embodiments, 5% or fewer of codons beginning with an adenosine nucleotide are immediately preceded by a codon ending in a cytidine nucleotide. In some embodiments, 4% or fewer of codons beginning with an adenosine nucleotide are immediately preceded by a codon ending in a cytidine nucleotide. In some embodiments, 3% or fewer of codons beginning with an adenosine nucleotide are immediately preceded by a codon ending in a cytidine nucleotide. In some embodiments, 2% or fewer of codons beginning with an adenosine nucleotide are immediately preceded by a codon ending in a cytidine nucleotide. In some embodiments, 1% or fewer of codons beginning with an adenosine nucleotide are immediately preceded by a codon ending in a cytidine nucleotide. In some embodiments, no codons beginning with an adenosine nucleotide are immediately preceded by a codon ending in a cytidine nucleotide.

In some embodiments, fewer than 30% of amino acids that immediately precede an isoleucine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede an isoleucine residue in the polypeptide are encoded by codons that end in a cytidine nucleotide. In some embodiments, no amino acid that immediately precedes an isoleucine residue in the polypeptide is encoded by a codon that ends in a cytidine nucleotide.

In some embodiments, fewer than 30% of amino acids that immediately precede a methionine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede an methionine residue in the polypeptide are encoded by codons that end in a cytidine nucleotide. In some embodiments, no amino acid that immediately precedes a methionine residue in the polypeptide is encoded by a codon that ends in a cytidine nucleotide.

In some embodiments, fewer than 30% of amino acids that immediately precede a threonine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede a threonine residue in the polypeptide are encoded by codons that end in a cytidine nucleotide. In some embodiments, no amino acid that immediately precedes a threonine residue in the polypeptide is encoded by a codon that ends in a cytidine nucleotide.

In some embodiments, fewer than 30% of amino acids that immediately precede an asparagine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede an asparagine residue in the polypeptide are encoded by codons that end in a cytidine nucleotide. In some embodiments, no amino acid that immediately precedes an asparagine residue in the polypeptide is encoded by a codon that ends in a cytidine nucleotide.

In some embodiments, fewer than 30% of amino acids that immediately precede a lysine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede a lysine residue in the polypeptide are encoded by codons that end in a cytidine nucleotide. In some embodiments, no amino acid that immediately precedes a lysine residue in the polypeptide is encoded by a codon that ends in a cytidine nucleotide.

In some embodiments, fewer than 30% of amino acids that immediately precede a serine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede a serine residue in the polypeptide are encoded by codons that end in a cytidine nucleotide. In some embodiments, no amino acid that immediately precedes a serine residue in the polypeptide is encoded by a codon that ends in a cytidine nucleotide.

In some embodiments, fewer than 30% of amino acids that immediately precede an arginine residue in the polypeptide are encoded by codons that end in a cytidine nucleotide. In some embodiments, 25% or fewer, 20% or fewer, 15% or fewer, 12% or fewer, 10% or fewer, 8% or fewer, 6% or fewer, 5% or fewer, 4% or fewer, 3% or fewer, 2% or fewer, or 1% or fewer of amino acids that immediately precede an arginine residue in the polypeptide are encoded by codons that end in a cytidine nucleotide. In some embodiments, no amino acid that immediately precedes an arginine residue in the polypeptide is encoded by a codon that ends in a cytidine nucleotide.

In some embodiments, no amino acid that immediately precedes an isoleucine, methionine, threonine, asparagine, or lysine residue in the polypeptide is encoded by a codon that ends in a cytidine nucleotide. In some embodiments, no amino acid that immediately precedes a serine or arginine in the polypeptide, where the serine or arginine is encoded by a codon beginning with an adenosine nucleotide, is encoded by a codon that ends in a cytidine nucleotide.

To reduce the number of CpA dinucleotides of an mRNA sequence, a codon comprising a CpA dinucleotide may be substituted with any synonymous codon (i.e., a codon encoding the same amino acid as the substituted codon) that does not comprise a CpA dinucleotide. Multiple codons comprising CpA dinucleotides may be substituted with the same synonymous codon, or with different synonymous codons. For example, two or more ACA codons may each be substituted with an ACU codon, or one ACA codon may be substituted with an ACC codon and another may be substituted with an ACG codon. Substituting multiple instances of the same codon with different synonymous codons may be useful, for example, to achieve a desired distribution of codons encoding a given amino acid in an mRNA sequence. In some embodiments, 50% or fewer, 40% or fewer, 30% or fewer, 25% or fewer, 20% or fewer, 15% or fewer, 10% or fewer, or 5% or fewer UCA codons are substituted with a UCC codon. In some embodiments, 50% or fewer, 40% or fewer, 30% or fewer, 25% or fewer, 20% or fewer, 15% or fewer, 10% or fewer, or 5% or fewer UCA codons are substituted with a UCG codon. In some embodiments, 5-75%, 10-60%, 15-50%, 20-40%, or 25-35% of UCA codons are substituted with a UCC codon. In some embodiments, 5-75%, 10-60%, 15-50%, 20-40%, or 25-35% of UCA codons are substituted with a UCG codon. In some embodiments, the modified mRNA sequence comprises an ORF in which 5-80%, 10-70%, 15-60%, 20-50%, 25-40%, or 25-35% of codons encoding serine residues are UCU codons. In some embodiments, the modified mRNA sequence comprises an ORF in which 5-80%, 10-70%, 15-60%, 20-50%, 25-40%, or 25-35% of codons encoding serine residues are UCC codons. In some embodiments, the modified mRNA sequence comprises an ORF in which 5-80%, 10-70%, 15-60%, 20-50%, 25-40%, or 25-35% of codons encoding serine residues are UCG codons.

In some embodiments, 50% or fewer, 40% or fewer, 30% or fewer, 25% or fewer, 20% or fewer, 15% or fewer, 10% or fewer, or 5% or fewer GCA codons are substituted with a GCC codon. In some embodiments, 50% or fewer, 40% or fewer, 30% or fewer, 25% or fewer, 20% or fewer, 15% or fewer, 10% or fewer, or 5% or fewer GCA codons are substituted with a GCG codon. In some embodiments, 5-75%, 10-60%, 15-50%, 20-40%, or 25-35% of GCA codons are substituted with a GCC codon. In some embodiments, 5-75%, 10-60%, 15-50%, 20-40%, or 25-35% of GCA codons are substituted with a GCG codon. In some embodiments, the modified mRNA sequence comprises an ORF in which 5-80%, 10-70%, 15-60%, 20-50%, 25-40%, or 25-35% of codons encoding alanine residues are GCU codons. In some embodiments, the modified mRNA sequence comprises an ORF in which 5-80%, 10-70%, 15-60%, 20-50%, 25-40%, or 25-35% of codons encoding alanine residues are GCC codons. In some embodiments, the modified mRNA sequence comprises an ORF in which 5-80%, 10-70%, 15-60%, 20-50%, 25-40%, or 25-35% of codons encoding alanine residues are GCG codons.

In some embodiments, 50% or fewer, 40% or fewer, 30% or fewer, 25% or fewer, 20% or fewer, 15% or fewer, 10% or fewer, or 5% or fewer ACA codons are substituted with a ACC codon. In some embodiments, 50% or fewer, 40% or fewer, 30% or fewer, 25% or fewer, 20% or fewer, 15% or fewer, 10% or fewer, or 5% or fewer ACA codons are substituted with a ACG codon. In some embodiments, 5-75%, 10-60%, 15-50%, 20-40%, or 25-35% of ACA codons are substituted with a ACC codon. In some embodiments, 5-75%, 10-60%, 15-50%, 20-40%, or 25-35% of ACA codons are substituted with a ACG codon. In some embodiments, the modified mRNA sequence comprises an ORF in which 5-80%, 10-70%, 15-60%, 20-50%, 25-40%, or 25-35% of codons encoding threonine residues are ACU codons. In some embodiments, the modified mRNA sequence comprises an ORF in which 5-80%, 10-70%, 15-60%, 20-50%, 25-40%, or 25-35% of codons encoding threonine residues are ACC codons. In some embodiments, the modified mRNA sequence comprises an ORF in which 5-80%, 10-70%, 15-60%, 20-50%, 25-40%, or 25-35% of codons encoding threonin residues are ACG codons.

In some embodiments, 50% or fewer, 40% or fewer, 30% or fewer, 25% or fewer, 20% or fewer, 15% or fewer, 10% or fewer, or 5% or fewer CCA codons are substituted with a CCC codon. In some embodiments, 50% or fewer, 40% or fewer, 30% or fewer, 25% or fewer, 20% or fewer, 15% or fewer, 10% or fewer, or 5% or fewer CCA codons are substituted with a CCG codon. In some embodiments, 5-75%, 10-60%, 15-50%, 20-40%, or 25-35% of CCA codons are substituted with a CCC codon. In some embodiments, 5-75%, 10-60%, 15-50%, 20-40%, or 25-35% of CCA codons are substituted with a CCG codon. In some embodiments, the modified mRNA sequence comprises an ORF in which 5-80%, 10-70%, 15-60%, 20-50%, 25-40%, or 25-35% of codons encoding proline residues are CCU codons. In some embodiments, the modified mRNA sequence comprises an ORF in which 5-80%, 10-70%, 15-60%, 20-50%, 25-40%, or 25-35% of codons encoding proline residues are CCC codons. In some embodiments, the modified mRNA sequence comprises an ORF in which 5-80%, 10-70%, 15-60%, 20-50%, 25-40%, or 25-35% of codons encoding proline residues are CCG codons.

In some embodiments, substituting multiple instances of a given codon with the same synonymous codon may be useful, for example, to achieve a desired property of an mRNA sequence (e.g., % G/C content). In some embodiments, one or more codons are substituted with codons comprising a higher % G/C content. In some embodiments, 50% or more, 60% or more, 70% or more, 80% or more, 90% or more, 95% or more, 97% or more, 98% or more, 99% or more, or up to 100% of UCA codons are substituted with codons comprising either UCC or UCG. In some embodiments, 50% or more, 60% or more, 70% or more, 80% or more, 90% or more, 95% or more, 97% or more, 98% or more, 99% or more, or up to 100% of CCA codons are substituted with codons comprising either CCC or CCG. In some embodiments, 50% or more, 60% or more, 70% or more, 80% or more, 90% or more, 95% or more, 97% or more, 98% or more, 99% or more, or up to 100% of ACA codons are substituted with codons comprising either ACC or ACG. In some embodiments, 50% or more, 60% or more, 70% or more, 80% or more, 90% or more, 95% or more, 97% or more, 98% or more, 99% or more, or up to 100% of GCA codons are substituted with codons comprising either GCC or GCG.

In some embodiments, one or more codons are substituted with codons comprising an equal % G/C content. In some embodiments, 50% or more, 60% or more, 70% or more, 80% or more, 90% or more, 95% or more, 97% or more, 98% or more, 99% or more, or up to 100% of UCA codons are substituted with UCU codons. In some embodiments, 50% or more, 60% or more, 70% or more, 80% or more, 90% or more, 95% or more, 97% or more, 98% or more, 99% or more, or up to 100% of CCA codons are substituted with CCU codons. In some embodiments, 50% or more, 60% or more, 70% or more, 80% or more, 90% or more, 95% or more, 97% or more, 98% or more, 99% or more, or up to 100% of ACA codons are substituted with ACU codons. In some embodiments, 50% or more, 60% or more, 70% or more, 80% or more, 90% or more, 95% or more, 97% or more, 98% or more, 99% or more, or up to 100% of GCA codons are substituted with CCU codons.

In addition to substituting codons to reduce the abundance of CpA dinucleotides in the ORF of an mRNA, CpA dinucleotide abundance may also be reduced by substituting nucleotides in untranslated regions (UTRs) of an mRNA, such as a 5′ UTR or 3′ UTR. The extent to which mRNA stability may be improved by substituting one or more nucleotides of the 5′ UTR or 3′ UTR depends on the abundance of CpA dinucleotides in the sequence of unmodified UTRs. In some embodiments, 50% or more, 60% or more, 70% or more, 75% or more, 80% or more, 85% or more, 90% or more, 95% or more, 97% or more, 98% or more, 99% or more, or up to 100% of CpA dinucleotides in a 5′ UTR are removed by substitution. In some embodiments, 50% or more, 60% or more, 70% or more, 75% or more, 80% or more, 85% or more, 90% or more, 95% or more, 97% or more, 98% or more, 99% or more, or up to 100% of CpA dinucleotides in a 3′ UTR are removed by substitution. Removing one or more CpA dinucleotides from an mRNA sequence may be achieved by substituting the cytidine nucleotide, the adenosine nucleotide, or both nucleotides of a CpA dinucleotide with different nucleotides, provided that the substitution does not introduce a new CpA dinucleotide into the sequence. For example, substituting the first adenosine nucleotide in the sequence CAA with a cytidine nucleotide would produce the sequence CCA, which contains the same number of CpA dinucleotides, and thus an alternative substitution would be required to reduce the number of CpA dinucleotides in this sequence.

In some embodiments of the modified mRNAs described herein or modified mRNA sequences produced by methods described herein, the modified mRNA comprises a 5′ UTR that does not comprise a CpA dinucleotide. In some embodiments, an mRNA described herein comprises a 3′ UTR that does not comprise a CpA dinucleotide. In some embodiments, the only CpA dinucleotides present in an mRNA sequence are located in codons encoding histidine or glutamine residues.

In some embodiments, an mRNA sequence comprises one or more CpA dinucleotides that are present in regulatory motifs. In some embodiments, the 5′ UTR comprises 5 or fewer, 4 or fewer, 3 or fewer, 2 or fewer, 1 or fewer, or 0 CpA dinucleotides. In some embodiments, the 5′ UTR comprises no more than five CpA dinucleotides. In some embodiments, the 5′ UTR comprises no more than four CpA dinucleotides. In some embodiments, the 5′ UTR comprises no more than three CpA dinucleotides. In some embodiments, the 5′ UTR comprises no more than two CpA dinucleotides. In some embodiments, the 5′ UTR comprises no more than one CpA dinucleotides. In some embodiments, the 5′ UTR does not comprise a CpA dinucleotide. In some embodiments, the 3′ UTR comprises 5 or fewer, 4 or fewer, 3 or fewer, 2 or fewer, 1 or fewer, or 0 CpA dinucleotides. In some embodiments, the 3′ UTR comprises no more than five CpA dinucleotides. In some embodiments, the 3′ UTR comprises no more than four CpA dinucleotides. In some embodiments, the 3′ UTR comprises no more than three CpA dinucleotides. In some embodiments, the 3′ UTR comprises no more than two CpA dinucleotides. In some embodiments, the 3′ UTR comprises no more than one CpA dinucleotides. In some embodiments, the 3′ UTR does not comprise a CpA dinucleotide. In some embodiments, the last nucleotide of the 5′ UTR (immediately preceding the AUG start codon) is not a cytidine nucleotide. In some embodiments, the last nucleotide of the 3′ UTR (immediately preceding the polyA tail) is not a cytidine nucleotide.

Some embodiments of mRNAs described herein, and modified mRNAs made by described methods, comprise a sequence with a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%. In some embodiments, the nucleic acid sequence of the full-length mRNA comprises a % G/C content of 30% to 80%, 40%-70%, 50%-60%, 35%-50%, 50%-65%, 65%-70%, 40%-45%, 45%-50%, 50%-55%, 55%-70%, 70%-75%, or 75%-80%. In some embodiments, the mRNA comprises an ORF with a % G/C content from about 30% to about 80%, about 35% to about 70%, about 40% to about 60%, about 45% to about 55%, about 40% to about 70%, about 50% to about 60%, about 35% to about 50%, about 50% to about 50% to about 65%, about 65% to about 70%, about 40% to about 45%, about 45% to about 50%, about 50% to about 55%, about 55% to about 70%, about 70% to about 75%, or about 75% to about 80%. In some embodiments, the mRNA comprises 5′ UTR with a % G/C content from about 30% to about 80%, about 35% to about 70%, about 40% to about 60%, about 45% to about 55%, about 40% to about 70%, about 50% to about 60%, about 35% to about 50%, about 50% to about 50% to about 65%, about 65% to about 70%, about 40% to about 45%, about 45% to about 50%, about 50% to about 55%, about 55% to about 70%, about 70% to about 75%, or about 75% to about 80%. In some embodiments, the mRNA comprises 3′ UTR with a % G/C content from about 30% to about 80%, about 35% to about 70%, about 40% to about 60%, about 45% to about 55%, about 40% to about 70%, about 50% to about 60%, about 35% to about 50%, about 50% to about 50% to about 65%, about 65% to about 70%, about 40% to about 45%, about 45% to about 50%, about 50% to about 55%, about 55% to about 70%, about 70% to about 75%, or about 75% to about 80%. In some embodiments, a modified mRNA made by a method described herein comprises a higher % G/C content than a reference mRNA sequence. In some embodiments, the % G/C content of the modified mRNA sequence is 2% or more, 3% or more, 4% or more, 5% or more, 6% or more, 7% or more, 8% or more, 9% or more, 10% or more, 12% or more, 15% or more, or 20% or more than the % G/C content of the reference RNA sequence. In some embodiments, the % G/C content of the modified ORF sequence is 2% or more, 3% or more, 4% or more, 5% or more, 6% or more, 7% or more, 8% or more, 9% or more, 10% or more, 12% or more, 15% or more, or 20% or more than the % G/C content of the reference ORF sequence. In some embodiments, the % G/C content of the modified 5′ UTR sequence is 2% or more, 3% or more, 4% or more, 5% or more, 6% or more, 7% or more, 8% or more, 9% or more, 10% or more, 12% or more, 15% or more, or 20% or more than the % G/C content of the reference 3′ UTR sequence.

Some embodiments of mRNAs described herein, and modified mRNAs made by described methods, express one or more encoded proteins in a mammalian cell at a level that is at least 50% of the level of expression of a reference mRNA encoding a protein with the same amino acid sequence, but containing a higher number of CpA dinucleotides. Expression of an encoded protein may refer to the number of copies of an encoded polypeptide produced by translation of a given mRNA molecule. Typically, a reduction in the level of an mRNA (e.g., by mRNA cleavage) results in a reduction in the level of a polypeptide translated therefrom. The level of expression may be determined using standard techniques for measuring protein. In some embodiments, an mRNA has a level of expression in a mammalian cell that is at least 60%, at least 70%, at least 75%, at least 80%, at least 85%, at least 90%, at least 95%, at least 97%, at least 98%, at least 99%, or at least 100% of the level of expression of a reference mRNA encoding a protein with the same amino acid sequence, but containing a higher number of CpA dinucleotides. Examples of mammalian cells for use in evaluating expression of an mRNA include, without limitation, humans, mice, rats, hamsters, guinea pigs, cats, dogs, chimpanzees, macaques, baboons, and gorillas. In some embodiments, the mammalian cell is a human cell.

Some embodiments of the mRNAs described herein or produced by a method described herein are stable for longer periods of time than reference mRNAs having higher numbers of CpA dinucleotides but encoding a protein with the same amino acid sequence. In some embodiments, the modified mRNA has a coefficient of degradation below a threshold value. As used herein, a “coefficient of degradation” refers to a parameter of an equation describing the loss of nucleic acid purity over time. As used herein, “nucleic acid purity” refers to the percentage of nucleic acid in a composition having a desired sequence and structure. Compositions may be prepared using nucleic acids having a specific sequence encoding a protein to be expressed in cells. During storage, the nucleic acid may be degraded by environmental factors such as water or nucleases. Water molecules can hydrolyze the phosphodiester bond that bridges a phosphate moiety and sugar moiety in the sugar-phosphate backbone of a nucleic acid, resulting in the production of two separate nucleic acid molecules, neither of which contains an intact sequence encoding the full-length protein encoded by the unhydrolyzed nucleic acid. Nucleases are enzymes that can facilitate this process, but nucleic acids are susceptible to degradation by water molecules even in the absence of environmental nucleases. Nucleic acid purity may be measured by any one of multiple methods known in the art, such as mass spectrometry or high-performance liquid chromatography (HPLC) (see, e.g., Papadoyannis et al., J Liq Chrom Relat Tech. 2007. 27 (6): 1083-1092). In HPLC, a sample to be analyzed, such as nucleic acid, is dissolved in a solvent (mobile phase) and passed through a column containing a solid material (stationary phase), with a detector measuring the presence of dissolved sample molecules as the mobile phase is eluted from the column. The rate at which molecules of the sample move through the stationary phase depends on multiple factors, including size, such that different components of the sample will be observed at different times. A sample containing 100% pure nucleic acid will produce a single peak (main peak) on a chromatogram when analyzed by HPLC, while a sample containing multiple different nucleic acid molecules will produce multiple peaks, including a main peak and one or more impurity peaks, for a total of N peaks. To calculate the purity of a nucleic acid using HPLC analysis, the area under the curve (A.U.C.) of each of N peaks is calculated by integration, and the percent purity is calculated using the equation

$$\% purity = \frac{AUC ( mean peak )}{{\sum }_{i = 1}^{N} AUC ( {peak}_{i} )} .$$

Loss of nucleic acid purity over time may be described by a differential equation of the form

$$\frac{dP}{dt} = - λ P ,$$

where P is nucleic acid purity (%) λ is the coefficient of degradation, and dP/dt is the rate of change in nucleic acid purity. Alternatively, nucleic acid purity over time may be described by an equation of the form P(t)=P₀ₑ^{-λt}, where P(t) is nucleic acid purity (%) at a given time, t, P₀ is initial nucleic acid purity at time t=0, e is the base of the natural logarithm, and λ is the coefficient of degradation. In both equation forms, a positive value of λ indicates exponential decay, while a negative λ indicates exponential growth, with larger absolute values of λ indicating faster decay or growth, respectively. In some embodiments, the coefficient of degradation is expressed in units of day⁻¹. In some embodiments, the modified mRNA has a coefficient of degradation at 25° C. that is 90% or less, 80% or less, 70% or less, 60% or less, or 50% or less, relative to an mRNA comprising a wild-type ORF encoding the polypeptide. In some embodiments, the coefficient of degradation of the modified mRNA at a temperature of 2° C.-8° C. is 90% or less, 80% or less, 70% or less, 60% or less, or 50% or less, relative to an mRNA comprising a wild-type ORF encoding the polypeptide. In some embodiments, the coefficient of degradation of the modified mRNA is 90% or less, relative to an mRNA comprising a wild-type ORF encoding the polypeptide. In some embodiments, the coefficient of degradation of the modified mnRNA is 80% or less, relative to an mRNA comprising a wild-type ORF encoding the polypeptide. In some embodiments, the coefficient of degradation of the modified mRNA is 70% or less, relative to an mRNA comprising a wild-type ORE encoding the polypeptide. In some embodiments, the coefficient of degradation of the modified mRNA is 60% or less, relative to an mRNA comprising a wild-type ORF encoding the polypeptide. In some embodiments, the coefficient of degradation of the modified mRNA is 50% or less, relative to an mRNA comprising a wild-type ORF encoding the polypeptide.

In some embodiments, the decrease in degradation coefficient is calculated with respect to storage of modified mRNAs in the absence of lipid nanoparticles. In some embodiments, the decrease in degradation coefficient is calculated with respect to storage of modified mRNAs in a buffer lacking lipid nanoparticles. In some embodiments, the buffer comprises 10-100 mM Tris. In some embodiments, the buffer comprises 5-10% sucrose. In some embodiments, the buffer has a pH of about 7.3 to about 7.6. In some embodiments, the buffer comprises 10-100 mM Tris, 5-10% sucrose, and has a pH of 7.3 to 7.6. In some embodiments, the decrease in degradation coefficient is calculated with respect to storage of mRNAs formulated in lipid nanoparticles. The lipid nanoparticles may be any lipid nanoparticle described herein. Alternatively, the lipid nanoparticles may be another lipid nanoparticle known in the art.

In some embodiments, reduction in degradation coefficient is measured in mRNAs having an ORF of a length in a specific range, as it is understood that the length of an mRNA affects stability during storage (e.g., shorter mRNAs are less susceptible to degradation than longer mRNAs). In some embodiments, the modified mRNA having a reduced degradation coefficient comprises an ORE that is 100-500, 500-1,000, 1,000-2,000, 2,000-3.000, 3,000-5,000, 100-5,000, 100-2,500, 100-1,500, 100-1.000, 500-5,000, 500-2,500, 500-1,000, 1,000-5,000, 1,000-4,000, 1,000-3,000, 1,000-2,000, 2,000-5.000, 2,000-5,000, or 3,000-4.000 nucleotides in length. In some embodiments, the modified mRNA having a reduced degradation coefficient comprises an ORE that is 300-5,000 nucleotides in length. In some embodiments, the modified mRNA having a reduced degradation coefficient comprises an ORF that is 300-1,500 nucleotides in length. In some embodiments, the modified mRNA having a reduced degradation coefficient comprises an ORF that is 1,500-3,000 nucleotides in length. In some embodiments, the modified mRNA having a reduced degradation coefficient comprises an ORF that is 3,000-5,000 nucleotides in length.

In some embodiments, the nucleic acid degrades (e.g., as measured by capillary electrophoresis) about 2% or less per month during storage, such as about 1% or less, about 0.75% or less, about 0.5% or less, about 0.4% or less, about 0.3% or less, about 0.2% or less, or about 0.1% or less per month during storage (e.g., at 4° C.). In some embodiments, the methods comprise producing compositions comprising modified nucleic acid, where the modified nucleic acid in the composition is at least 50% pure (such as about 50% pure, about 55% pure, about 60% pure, about 65% pure, about 70% pure, or about 75% pure or more) after storage at 0° C. or more (such as 0° C., 2° C., 4° C. 5° C., 8° C., 10° C., 15° C., 20° C., 25° C., or 2-8° C.) for a given length of time. The length of time for which a composition will comprise at least 50% pure nucleic acid can be predicted by measuring a) the initial purity of the nucleic acid in a composition, and b) the coefficient of degradation of nucleic acid, as described above, then using the equation P(t)=P₀ₑ^{-λt} to calculate the value of t at which P(t)=50% or 0.5. This length of time is given by the formula

$$t = \frac{ln 50 \% - ln {P}_{0}}{- λ}$$

if P₀ is expressed as a percentage or

$$t = \frac{ln 0.5 - ln {P}_{0}}{- λ}$$

if P₀ is expressed as a proportion.

In some embodiments, a composition comprising a plurality of the modified mRNAs remains above 50% purity (such as about 50% pure, about 55% pure, about 60% pure, about 65% pure, about 70% pure, or about 75% pure or more) for at least 30 days, at least 40 days, at least 50 days, at least 60 days, at least 75 days, at least 90 days, at least 120 days, at least 150 days, or at least 180 days longer in storage than a composition comprising a plurality of mRNA comprising a wild-type ORF encoding the polypeptide. In some embodiments, the increase in duration of maintenance above 50% purity is during storage of modified mRNAs in the absence of lipid nanoparticles. In some embodiments, the increase in duration of maintenance above 50% purity is during storage of modified mRNAs in a buffer lacking lipid nanoparticles. In some embodiments, the buffer comprises 10-100 mM Tris. In some embodiments, the buffer comprises 5-10% sucrose. In some embodiments, the buffer has a pH of about 7.3 to about 7.6. In some embodiments, the buffer comprises 10-100 mM Tris, 5-10% sucrose, and has a pH of 73 to 76. In some embodiments, the increased duration of maintenance above 50% purity is during storage of mRNAs formulated in lipid nanoparticles. The lipid nanoparticles may be any lipid nanoparticle described herein. Alternatively, the lipid nanoparticles may be another lipid nanoparticle known in the art. In some embodiments, improved stability is measured in mRNAs having an ORF of a length in a specific range, as it is understood that the length of an mRNA affects stability during storage (e.g., longer mRNAs are less stable than shorter mRNAs). In some embodiments, the mRNA having improved stability comprises an ORF that is 100-500, 500-1,000, 1,000-2,000, 2,000-3,000, 3,000-5,000, 100-5,000, 100-2,500, 100-1,500, 100-1,000, 500-5,000, 500-2,500, 500-1,000, 1,000-5,000, 1,000-4,000, 1,000-3,000, 1,000-2,000, 2,000-5,000, 2,000-5,000, or 3,000-4,000 nucleotides in length. In some embodiments, the mRNA having improved stability comprises an ORF that is 300-5,000 nucleotides in length. In some embodiments, the mRNA having improved stability comprises an ORF that is 300-1,500 nucleotides in length. In some embodiments, the mRNA having improved stability comprises an ORF that is 1,500-3,000 nucleotides in length. In some embodiments, the mRNA having improved stability comprises an ORF that is 3,000-5,000 nucleotides in length.

In some embodiments, the storage is conducted at a temperature between about 2° C. and about 40° C. In some embodiments, the storage is conducted at a temperature between about 22° C. and about 28° C. In some embodiments, the storage is conducted at about 25° C. In some embodiments, the storage is conducted at a temperature between about 2° C. and about 15° C. In some embodiments, the storage is conducted at a temperature between about 2° C. and about 8° C. In some embodiments, the storage is conducted at about 3° C. In some embodiments, the storage is conducted at about 5° C. Degradation of nucleic acids is a chemical reaction that occurs more readily at higher temperatures, and as such the coefficient of degradation and kinetics of purity depend on the temperature at which nucleic acids are stored.

In some embodiments, the stability of a modified mRNA is evaluated by storing the mRNA in a buffer with a defined composition. In some embodiments, the mRNA is stored in a buffer comprising 10-100 mM Tris. In some embodiments, the mRNA is stored in a buffer comprising 5-10% sucrose. In some embodiments, the mRNA is stored in a buffer having a pH of about 7.3 to about 7.6. In some embodiments, the storage buffer comprises 10-100 mM Tris, 5-10% sucrose, and a pH of 7.3 to 7.6.

### Codon Optimization

In some embodiments, an mRNA is codon-optimized. Codon optimization methods are known in the art. Codon optimization, in some embodiments, may be used to match codon frequencies in target and host organisms to ensure proper folding; bias % G/C content to increase mRNA thermodynamic stability or reduce secondary structures: minimize tandem repeat codons or base runs that may impair gene construction or expression; customize transcriptional and translational control regions; insert or remove protein trafficking sequences: remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding the polypeptide). In some embodiments, a codon optimized sequence shares less than 90% sequence identity to a naturally-occurring or wild-type sequence (e.g., a naturally-occurring or wild-type mRNA sequence encoding the polypeptide). In some embodiments, a codon optimized sequence shares less than 85% sequence identity to a naturally-occurring or wild-type sequence (e.g., a naturally-occurring or wild-type mRNA sequence encoding the polypeptide) In some embodiments, a codon optimized sequence shares less than 80% sequence identity to a naturally-occurring or wild-type sequence (e.g., a naturally-occurring or wild-type mRNA sequence encoding the polypeptide). In some embodiments, a codon optimized sequence shares less than 75% sequence identity to a naturally-occurring or wild-type sequence (e.g., a naturally-occurring or wild-type mRNA sequence encoding the polypeptide).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding the polypeptide). In some embodiments, a codon optimized sequence shares between 65% and 75% or about 80% sequence identity to a naturally-occurring or wild-type sequence (e.g., a naturally-occurring or wild-type mRNA sequence encoding the polypeptide).

When transfected into mammalian host cells, some embodiments of modified mRNAs have a stability of between 12-18 hours, or greater than 18 hours, e.g., 24, 36, 48. 60, 72, or greater than 72 hours and are capable of being expressed by the mammalian host cells.

In some embodiments, a codon optimized RNA may be one in which the levels of CC are enhanced. The C/C-content of nucleic acid molecules (e.g., mRNA) may influence the stability of the RNA. RNA having an increased amount of guanine (C) and/or cytosine (C) residues may be more thermodynamically stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, one or more cytidine or adenosine nucleotides of a CpA dinucleotide comprises a modified nucleotide. In some embodiments, one or more cytidine nucleotides of a CpA dinucleotide comprises a modified nucleotide. Without wishing to be bound by any particular theory, it is believed that the substitution of a conventional cytidine or adenosine nucleotide for a modified cytidine or adenosine nucleotide, respectively, is useful for reducing the susceptibility of the internucleoside linkage of a CpA dinucleotide to hydrolysis. Such substitutions are useful, for example, to improve mRNA stability where CpA dinucleotides are necessary, such as in codons encoding histidine or glutamine or in regulatory motifs (e.g., Kozak sequence) In some embodiments, 10% or more, 20% or more, 30% or more, 40% or more, 50% or more, 60% or more, 70% or more, 80% or more, 90% or more, 95% or more, 97% or more, 98% or more. 99% or more, or up to 100% of CpA dinucleotides in a modified mRNA sequence comprise a modified cytidine nucleotide and/or a modified adenosine nucleotide, in some embodiments. 10% or more, 20% or more. 30% or more, 40% or more, 50% or more, 60% or more, 70% or more, 80% or more, 90% or more, 95% or more, 97% or more, 98% or more, 99% or more, or up to 100% of CpA dinucleotides in a modified mRNA sequence comprise a modified cytidine nucleotide. In some embodiments, 10% or more, 20% or more. 30% or more, 40% or more, 50% or more, 60% or more, 70% or more, 80% or more, 90% or more, 95% or more, 97% or more, 98% or more, 99% or more, or up to 100% of CpA dinucleotides in a modified mRNA sequence comprise a modified adenosine nucleotide. In some embodiments, 10% or more, 20% or more, 30% or more, 40% or more, 50% or more, 60% or more, 70% or more. 80% or more, 90% or more, 95% or more, 97% or more, 98% or more, 99% or more, or up to 100% of CpA dinucleotides in a modified mRNA sequence comprise a modified cytidine nucleotide and a modified adenosine nucleotide.

Multiple cytidine nucleotides may be substituted with the same or different modified cytidine nucleotides, and multiple adenosine nucleotides may be substituted with the same or different modified adenosine nucleotides. A modified cytidine nucleotide refers to a nucleotide comprising a structure different from the conventional structure of cytidine monophosphate (CMP) in an mRNA, but is still capable of hydrogen bonding with guanine (e.g., guanine of a guanosine nucleotide on a tRNA). A modified adenosine nucleotide refers to a nucleotide comprising a structure different from the conventional structure of adenosine monophosphate (AMP) in an mRNA, but is still capable of hydrogen bonding with uracil (e.g., uracil of a uridine nucleotide on a tRNA). A modified cytidine nucleotide may comprise a modified cytosine nucleobase (i.e., nucleobase that is capable of hydrogen bonding with guanine but has a different structure than canonical cytosine), a modified sugar (i.e., sugar other than ribose), and/or a modified phosphate (i.e., internucleoside linkage different from the canonical phosphate structure). Similarly, a modified adenosine nucleotide may comprise a modified adenine nucleobase (i.e., nucleobase that is capable of hydrogen bonding with uracil but has a different structure than canonical adenine), a modified sugar, and/or a modified phosphate. Non-limiting examples of modified nucleotides, including examples of modified nucleobases, modified sugars, and modified phosphates, are described in the section below entitled “Nucleic acids.”

### Nucleic Acids

Some aspects relate to compositions comprising nucleic acids and methods of producing nucleic acids. As used herein, the term “nucleic acid” includes multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). The term nucleic acid includes polyribonucleotides as well as polydeoxyribonucleotides. The term nucleic acid also includes polynucleoside (i.e., a polynucleotide minus the phosphate) and any other organic base containing polymer. Non-limiting examples of nucleic acids include chromosomes, genomic loci, genes, or gene segments that encode polynucleotides or polypeptides, coding sequences, non-coding sequences (e.g., intron, 5′-UTR, or 3-UTR) of a gene, pre-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA les one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acids described herein may include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids, or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses, and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5 to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate: a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an TVT reaction, as described herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, an RNA transcript (e.g., mRNA transcript) described herein may include a modified nucleobase selected from pseudouracil (ψ), N1-methylpseudouracil (m1ψ), 1-ethylpseudouracil, 2-thiouracil, 4′-thiouracil, 2-thio-1-methyl-1-deaza-pseudouracil, 2-thio-1-methyl-pseudouracil, 2-thio-5-aza-uracil, 2-thio-dihydropseudouracil, 2-thio-dihydrouracil, 2-thio-pseudouracil, 4-methoxy-2-thio-pseudouracil, 4-methoxy-pseudouracil, 4-thio-1-methyl-pseudouracil, 4-thio-pscudouracil, 5-aza-uracil, dihydropscudouracil, 5-methyluracil, 5-methoxyuracil (mo5U) and 2′-O-methyluracil. In some embodiments, an RNA transcript may include a modified cytosine nucleobase selected from digoxigeninated cytosine, 2-thiocytosine, 5-aminoallylcytosine, 5-bromocytosine, 5-carboxycytosine, 5-formylcytosine, 5-hydroxycytosine, 5-hydroxymethylcytosine, 5-methoxycytosine, 5-methylcytosine, 5-propargylaminocytosine, 5-propynylcytosine, 6-azacytosine, aracytosine, cyanine 3-5-propargylaminocytosine, cyanine 3-aminoallylcytosine, cyanine 5-6-propargylaminocytosine, cyanine 5-aminoallylcytosine, desthiobiotin-6-aminoallylcytosine, N4-biotin-OBEA-cytosine, N4-methylcytosine, pseudoisocytosine, and thienocytosine. In some embodiments, an RNA transcript may include a modified adenine nucleobase selected from digoxigeninated adenine, N6-methyladenine, 7-deazaadenine, 7-dcaza-7-propargylaminoadenine, 8-azaadenine, 8-azidoadenine, 8-chloroadenine, 8-oxoadenine, araadenine, N1-methyladenine, N6-methyladenine

3-deazaadenine, 2,6-diaminoadenine, 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (16A), 2-methylthio-N6-threonyl carbamoyladenine (ms216A), N6-methyl-N6-threonylcarbamoyladenine (m616A), N6-hydroxynorvalylcarbamoyladeninc (hn6A), 2-methylthio-N6-hydroxynorvalyl carbamoyladenine (ms2hn6A), N6,N6-dimethyladenine (m62A), and N6-acetyladenine (ac6A). In some embodiments, an RNA transcript (e.g., mRNA transcript) includes a combination of at least two (e.g., 2, 3, 4 or more) of the foregoing modified nucleobases.

Modified nucleotides may include modified sugars. For example, an RNA transcript (e.g., mRNA transcript) described herein may include a modified sugar selected from 2′-thioribose, 2′, 3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′, 3′-dideoxyribose, 3′-azido-2′, 3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-0,4′-C-methylene-linked, 2′-0,4′-C-amino-linked ribose, and 2′-0,4′-C-thio-linked ribose. In some embodiments, an RNA transcript (e.g., mRNA transcript) includes a combination of at least two (e.g., 2, 3, 4 or more) of the foregoing modified sugars.

Modified nucleotides may include modified phosphates. A modified phosphate group is a phosphate group that differs from the canonical structure of phosphate. An example of a canonical structure of a phosphate is shown below:

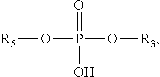

where R₅ and R³ are atoms or molecules to which the canonical phosphate is bonded. For example, for a phosphate in a nucleic acid sequence, R₅ may refer to the upstream nucleotide of the nucleic acid, and R³ may refer to the downstream nucleotide of the nucleic acid. The canonical structure of phosphate also refers to structures in which one or more hydroxyl groups of the phosphate are deprotonated, or in which an oxygen atom of the phosphate is bonded to an adjacent nucleotide in a nucleic acid sequence. In some embodiments, an RNA transcript (e.g., mRNA transcript) described herein may include a modified phosphate selected from phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, an RNA transcript (e.g., mRNA transcript) includes a combination of at least two (e.g., 2, 3, 4 or more) of the foregoing modified phosphates.

mRNAs described herein may be used to produce polypeptides of interest, such as therapeutic proteins and/or vaccine antigens. In some embodiments, an mRNA encodes a vaccine antigen. In some embodiments, an mRNA encodes a therapeutic protein. In some embodiments, the encoded polypeptide comprises 9-10,000, 9-9,000, 9-8,000, 9-7,000, 9-6,000, 9-5,000, 9-4,000, 9-3,000, 9-2,000, 9-1,000, 9-500, 9-400, 9-300, 9-200, 9-100, 9-10,000, 100-9,000, 100-8,000, 100-7,000, 100-6,000, 100-5,000, 100-4,000, 100-3,000, 100-2,000, 100-1,000, 100-500, 100-400, 100-300, 100-200, 100-9,000, 200-10,000, 200-9,000 200-8,000, 200-7,000, 200-6,000, 200-5,000, 200-4,000, 200-3,000, 200-2,000, 200-1,000, 200-500, 200-400, 500-10,000, 500-9,000, 500-8,000, 500-7,000, 500-6,000, 500-5,000, 500-4,000, 500-3,000, 500-2,000, 500-1,000, 1,000-10,000, 1,000-9,000, 1,000-8,000, 1,000-7,000, 1,000-6,000, 1,000-5,000, 1,000-4,000, 1,000-3,000, or 1,000-2,000 amino acids. In some embodiments, the encoded polypeptide consists of 9-10,000, 9-9,000, 9-8,000, 9-7,000, 9-6,000, 9-5,000, 9-4,000, 9-3,000, 9-2,000, 9-1,000, 9-500, 9-400, 9-300, 9-200, 9-100, 9-10,000, 100-9,000, 100-8,000, 100-7,000, 100-6,000, 100-5,000, 100-4,000, 100-3,000, 100-2,000, 100-1,000, 100-500, 100-400, 100-300, 100-200, 100-9,000, 200-10,000, 200-9,000 200-8,000, 200-7,000, 200-6,000, 200-5,000, 200-4,000, 200-3,000, 200-2,000, 200-1,000, 200-500, 200-400, 500-10,000, 500-9,000, 500-8,000, 500-7,000, 500-6,000, 500-5,000, 500-4,000, 500-3,000, 500-2,000, 500-1,000, 1,000-10,000, 1,000-9,000, 1,000-8,000, 1,000-7,000, 1,000-6,000, 1,000-5,000, 1,000-4,000, 1,000-3,000, or 1,000-2,000 amino acids. In some embodiments, the encoded polypeptide comprises 9-5,000 amino acids. In some embodiments, the encoded polypeptide consists of 9-5,000 amino acids. In some embodiments, the encoded polypeptide comprises 20-4,000 amino acids. In some embodiments, the encoded polypeptide consists of 20-4,000 amino acids. In some embodiments, the encoded polypeptide comprises 30-3,000 amino acids. In some embodiments, the encoded polypeptide consists of 30-3,000 amino acids. In some embodiments, the encoded polypeptide comprises 40-2,000 amino acids. In some embodiments, the encoded polypeptide consists of 40-2,000 amino acids. In some embodiments, the encoded polypeptide comprises 50-1,500 amino acids. In some embodiments, the encoded polypeptide consists of 50-1,500 amino acids. In some embodiments, the encoded polypeptide comprises 100-5,000 amino acids. In some embodiments, the encoded polypeptide consists of 100-5,000 amino acids. In some embodiments, the encoded polypeptide comprises 200-4,000 amino acids. In some embodiments, the encoded polypeptide consists of 200-4,000 amino acids. In some embodiments, the encoded polypeptide comprises 300-3,000 amino acids. In some embodiments, the encoded polypeptide consists of 300-3,000 amino acids. In some embodiments, the encoded polypeptide comprises 400-2,000 amino acids. In some embodiments, the encoded polypeptide consists of 400-2,000 amino acids. In some embodiments, the encoded polypeptide comprises 500-1,500 amino acids. In some embodiments, the encoded polypeptide consists of 500-1,500 amino acids.

A therapeutic mRNA is an mRNA that encodes a therapeutic protein (the term ‘protein’ encompasses peptides). In some embodiments, RNA compositions described herein comprise one or more RNAs that encode peptides or proteins that interact or complex in a cell or subject to form a multi-subunit protein (e.g., an antibody comprising a heavy chain and a light chain, a multi-subunit receptor protein, a multi-subunit signaling protein, a multi-subunit antigen, etc.) or a multivalent vaccine.

Therapeutic proteins mediate a variety of effects in a host cell or in a subject to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders. Other diseases and conditions are encompassed herein.

A protein or proteins of interest encoded by an RNA composition as described herein can be essentially any protein or peptide (e.g., peptide antigen).

In some embodiments, a therapeutic peptide or therapeutic protein is a biologic. A biologic is a polypeptide-based molecule that may be used to treat, cure, mitigate, prevent, or diagnose a serious or life-threatening disease or medical condition. Biologics include, but are not limited to, allergenic extracts (e.g. for allergy shots and tests), blood components, gene therapy products, human tissue or cellular products used in transplantation, vaccines, monoclonal antibodies, cytokines, growth factors, enzymes, thrombolytics, and immunomodulators, among others.

In some embodiments, the therapeutic protein is a cytokine, a growth factor, an antibody (e.g., monoclonal antibody), a fusion protein, or a vaccine (e.g., an RNA encoding one or more peptide antigens designed to elicit an immune response in a subject). Non-limiting examples of therapeutic proteins include blood factors (such as Factor VIII and Factor VII), complement factors, Low Density Lipoprotein Receptor (LDLR) and MUTI. Non-limiting examples of cytokines include interleukins, interferons, chemokines, lymphokines and the like. Non-limiting examples of growth factors include erythropoietin, EGFs, PDGFs, FGFs, TGFs, IGFs, TNFs, CSFs, MCSFs, GMCSFs and the like. Non-limiting examples of antibodies include adalimumab, infliximab, rituximab, ipilimumab, tocilizumab, canakinumab, itolizumab, tralokinumab, anti-influenza virus monoclonal antibody, anti-Chikungunya virus monoclonal antibody, anti-Zika virus monoclonal antibody, anti-SARS-COV-2 monoclonal antibody. Non-limiting examples of fusion proteins include, for example, etanercept, abatacept and belatacept. Non-limiting examples of multivalent vaccines include, for example, multivalent cytomegalovirus (CMV) vaccine, and personalized cancer vaccines.

One or more biologics currently being marketed or in development may be encoded by the RNA. While not wishing to be bound by theory, it is believed that incorporation of the encoding polynucleotides of a known biologic into the RNA described herein will result in improved therapeutic efficacy due at least in part to the specificity, purity and/or selectivity of the construct designs.

An RNA composition described herein may encode one or more antibodies (e.g., may comprise a first mRNA encoding an antibody heavy chain and a second RNA encoding an antibody light chain). The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. The term “immunoglobulin” (Ig) is used interchangeably with “antibody” herein. A monoclonal antibody is an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site.

Monoclonal antibodies specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity. Chimeric antibodies include, but are not limited to, “primatized” antibodies comprising variable domain antigen-binding sequences derived from a non-human primate (e.g., Old World Monkey, Ape etc.) and human constant region sequences.

Antibodies encoded in the RNA compositions may be utilized to treat conditions or diseases in many therapeutic areas such as, but not limited to, blood, cardiovascular, CNS, poisoning (including antivenoms), dermatology, endocrinology, gastrointestinal, medical imaging, musculoskeletal, oncology, immunology, respiratory, sensory and anti-infective.

An RNA composition described herein may encode one or more vaccine antigens. A vaccine antigen is a biological preparation that improves immunity to a particular disease or infectious agent. One or more vaccine antigens currently being marketed or in development may be encoded by the RNA. Vaccine antigens encoded in the RNA may be utilized to treat conditions or diseases in many therapeutic areas such as, but not limited to, cancer, allergy, and infectious disease. In some embodiments, a vaccine may be a personalized vaccine in the form of a concatemer or individual RNAs encoding peptide epitopes or a combination thereof.

An RNA composition described herein may be designed to encode on or more antimicrobial peptides (AMP) or antiviral peptides (AVP). AMPs and AVPs have been isolated and described from a wide range of animals such as, but not limited to, microorganisms, invertebrates, plants, amphibians, birds, fish, and mammals. The anti-microbial polypeptides may block cell fusion and/or viral entry by one or more enveloped viruses (e.g., HIV, HCV). For example, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the transmembrane subunit of a viral envelope protein, e.g., HIV-1 gp120 or gp41. The amino acid and nucleotide sequences of HIV-1 gp120 or gp41 are described in, e.g., Kuiken et al., (2008). “HIV Sequence Compendium,” Los Alamos National Laboratory.

In some embodiments, RNA transcripts (e.g., mRNA) are used for in vitro translation and microinjection. In some embodiments, RNA transcripts are used for RNA structure, processing and catalysis studies. In some embodiments, RNA transcripts are used for RNA amplification. In some embodiments, RNA transcripts are used as anti-sense RNA for gene expression modulation. Other applications are also encompassed.

### 5′ Cap Structures

In some embodiments, a composition includes an RNA polynucleotide having an open reading frame encoding at least one polypeptide having at least one modification, at least one 5′ terminal cap.

5′ terminal caps can include endogenous caps or cap analogs. A 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with a RNA polymerase variant, nucleoside triphosphates, and a cap analog under in vitro transcription reaction conditions to produce RNA transcript.

In some embodiments, the cap analog binds to a polynucleotide template that comprises a promoter region comprising a transcriptional start site having a first nucleotide at nucleotide position +1, a second nucleotide at nucleotide position +2, and a third nucleotide at nucleotide position +3. In some embodiments, the cap analog hybridizes to the polynucleotide template at least at nucleotide position +1, such as at the +1 and +2 positions, or at the +1, +2, and +3 positions.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise additional nucleotides 3′ of the inverted G,. e.g., 1, 2, or more nucleotides 3′ of the inverted G and 5′ to the 5′ UTR.

Exemplary caps comprise a sequence GG, GA, or GGA wherein the underlined, italicized G is an inverted G.

In some embodiments, a trinucleotide cap comprises a compound of Formula (III) or (IV), or a stereoisomer, tautomer, or salt thereof.

### Formula (III)

As described herein, a trinucleotide cap, in some embodiments, comprises a compound of formula (III):

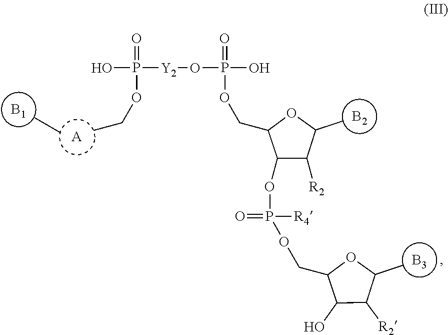

or a stereoisomer, tautomer, or salt thereof, wherein

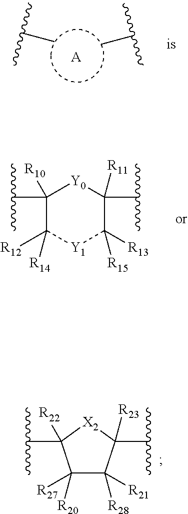

ring B₁ is a modified or unmodified Guanine;
ring B₂ and ring B₃ each independently is a nucleobase or a modified nucleobase;
X₂ is O, S(O)ₚ, NR²⁴ or CR²⁵R²⁶ in which p is 0, 1, or 2;
Y₀ is O or CR₆R₇;
Y₁ is O, S(O)ₙ, CR₆R₇, or NR₈, in which n is 0, 1, or 2;
each [symbol] is a single bond or absent, wherein when each [symbol] is a single bond, Yi is O, S(O)ₙ, CR₆R₇, or NR₈; and when each [symbol] is absent, Y₁ is void;
Y₂ is (OP(O)R⁴) m in which m is 0, 1, or 2, or —O—(CR⁴⁰R⁴¹)u-Q₀—(CR⁴²R⁴³)v-, in which Q₀ is a bond, O, S(O)ₙ, NR⁴⁴, or CR⁴⁵R⁴⁶, r is 0, 1, or 2, and each of u and v independently is 1, 2, 3 or 4;
each R² and R²′ independently is halo, LNA, or OR³;
each R³ independently is H, C₁-C₆ alkyl, C₂-C₆ alkenyl, or C₂-C₆ alkynyl and R³, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R⁴ and R⁴′ independently is H, halo, C₁-C₆ alkyl, OH, SH, SeH, or BH₃⁻;
each of R₆, R₇, and R₅, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR³¹ R³², (NR³¹R³²R³³)⁺, 4 to 12-membered heterocycloalkyl, or 5-or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR³¹R³², (NR³¹R³²R³³)⁺, C₃-C₈ cycloalkyl, C₆-C₁₀ aryl, 4 to 12-membered heterocycloalkyl, and 5-or 6-membered heteroaryl;

each of R¹⁰, R¹, R¹², R¹³ R¹⁴, and R¹⁵, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR³¹R³², (NR³¹R³²R³³)⁺, 4 to 12-membered heterocycloalkyl, or 5-or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR³¹R³², (NR³¹R³²R³³)⁺, C₃-C₈ cycloalkyl, C₆-C₁₀ aryl, 4 to 12-membered beterocycloalkyl, and 5-or 6-membered heteroaryl; or alternatively R¹² together with R¹⁴ is oxo, or R¹³ together with R¹⁵ is oxo, each of R²⁰, R²¹, R²², and R²³ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, R_{S3}, or OR_{S3}, in which R_{S3} is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5-or 6-membered heteroaryl, and R_{S3} is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5-or 6-membered heteroaryl; each of R²⁴, R²⁵, and R²⁶ independently is H or C₁-C₆ alkyl;
each of R²⁷ and R²⁸ independently is H or OR²⁹; or R²⁷ and Res together form O—R³⁰—O; each R²⁹ independently is H, C₁-C₆ alkyl, C₂-C₆ alkenyl, or C₂-C₆ alkynyl and R²⁹, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R³⁰ is C₁-C₆ alkylene optionally substituted with one or more of halo, OH and C₁-C₆ alkoxyl;
each of R³¹, R³², and R³³, independently is H, C₁-C₆ alkyl, C₃-C₈ cycloalkyl, C₆-C₁₀ aryl, 4 to 12-membered heterocycloalkyl, or 5-or 6-membered heteroaryl;
each of R⁴⁰, R⁴¹, R⁴², and R⁴³ independently is H, halo, OH, cyano, N₃, OP(O)R⁴⁷R⁴⁸, or C₁-C₆ alkyl optionally substituted with one or more OP(O)R⁴⁷R⁴⁸, or one R⁴¹ and one R⁴³, together with the carbon atoms to which they are attached and Q₀, form C₄-C₁₀ cycloalkyl, 4-to 14-membered heterocycloalkyl, C₆-C₁₀ aryl, or 5-to 14-membered heteroaryl, and each of the cycloalkyl, heterocycloalkyl, phenyl, or 5-to 6-membered heteroaryl is optionally substituted with one or more of OH, halo, cyano, N₃, oxo, OP(O)R⁴⁷R⁴⁸, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R⁴⁴ is H, C₁-C₆ alkyl, or an amine protecting group;
each of R⁴⁵ and R⁴⁶ independently is H, OP(O)R⁴⁷R⁴⁸, or C₁-C₆ alkyl optionally substituted with one or more OP(O)R⁴⁷R⁴⁸, and
each of R⁴⁷ and R⁴⁸, independently is H, halo, C₁-C₆ alkyl, OH, SH, SCH, or BH₃⁻.

It should be understood that a cap analog, as provided herein, may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R² is ethyl-based.

Thus, in some embodiments, a trinucleotide cap comprises the following structure:

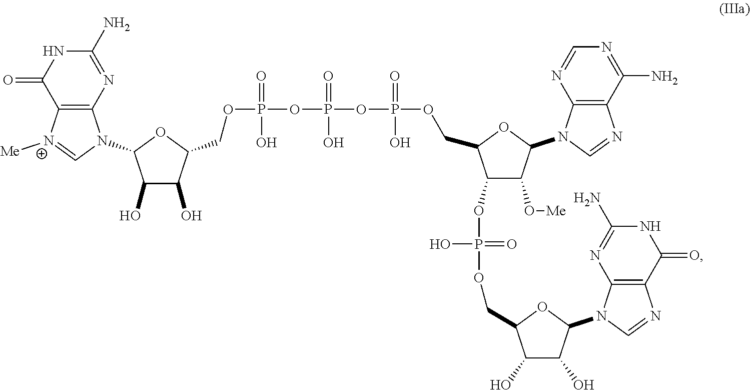 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)(O)OP(=O)(O)OP(=O)(O)OC[C@H]2O[C@@H](n3c[n+](C)c4c(=O)[nH]c(N)nc43)[C@H](O)[C@@H]2O)O[C@H]1n1cnc2c(N)ncnc21]

or a stereoisomer, tautomer, or salt thereof.

In yet other embodiments, a trinucleotide cap comprises the following structure:

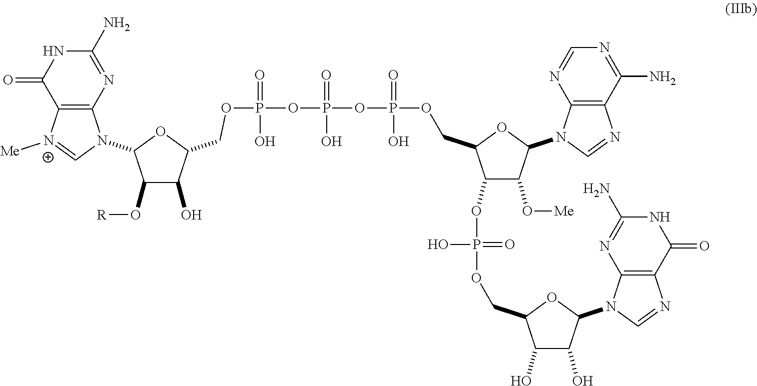

or a stereoisomer, tautomer or salt thereof.

In still other embodiments, a trinucleotide cap comprises the following structure:

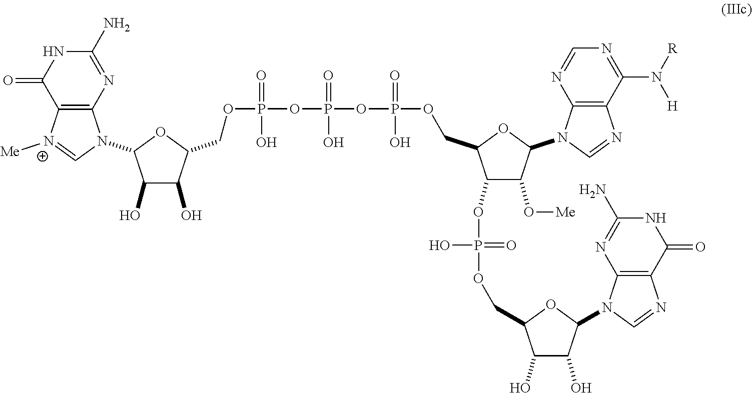

or a stereoisomer, tautomer, or salt thereof.

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises GAA. In some embodiments, a trinucleotide cap comprises GAC. In some embodiments, a trinucleotide cap comprises GAG. In some embodiments, a trinucleotide cap comprises GAU. In some embodiments, a trinucleotide cap comprises GCA. In some embodiments, a trinucleotide cap comprises GCC. In some embodiments, a trinucleotide cap comprises GCG. In some embodiments, a trinucleotide cap comprises GCU. In some embodiments, a trinucleotide cap comprises GGA. In some embodiments, a trinucleotide cap comprises GGC. In some embodiments, a trinucleotide cap comprises GGG. In some embodiments, a trinucleotide cap comprises GGU. In some embodiments, a trinucleotide cap comprises GUA. In some embodiments, a trinucleotide cap comprises GUC. In some embodiments, a trinucleotide cap comprises GUG. In some embodiments, a trinucleotide cap comprises GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m³GpppUpA, m⁷GpppUpC, m⁷GpppUpG, and m⁷GpppUpU.

In some embodiments, a trinucleotide cap comprises m⁷GpppApA. In some embodiments, a trinucleotide cap comprises m⁷GpppApC. In some embodiments, a trinucleotide cap comprises m⁷GpppApG. In some embodiments, a trinucleotide cap comprises m⁷GpppApU. In some embodiments, a trinucleotide cap comprises m⁷GpppCpA. In some embodiments, a trinucleotide cap comprises m⁷GpppCpC. In some embodiments, a trinucleotide cap comprises m⁷GpppCpG. In some embodiments, a trinucleotide cap comprises m⁷GpppCpU. In some embodiments, a trinucleotide cap comprises m⁷GpppGpA. In some embodiments, a trinucleotide cap comprises m⁷GpppGpC. In some embodiments, a trinucleotide cap comprises m⁷GpppGpG. In some embodiments, a trinucleotide cap comprises m⁷GpppGpU. In some embodiments, a trinucleotide cap comprises m⁷GpppUpA. In some embodiments, a trinucleotide cap comprises m⁷GpppUpC. In some embodiments, a trinucleotide cap comprises m⁷GpppUpG. In some embodiments, a trinucleotide cap comprises m⁷GpppUpU.

A trinucleotide cap, in some embodiments, comprises a sequence selected from the following sequences: m⁷g_{3′OMe}pppApA, m⁷g_{3′OMe}pppApC, m⁷g_{3′OMe}pppApG, m⁷g_{3′OMe}pppApU, m⁷g_{3′OMe}pppCpA, m⁷g_{3′OMe}pppCpC, m⁷g_{3′OMe}pppCpG, m⁷g_{3′OMe}pppCpU, m⁷g_{3′OMe}pppGpA, m⁷g_{3′OMe}pppGpC, m⁷g_{3′OMe}pppGpG, m⁷g_{3′OMe}pppGpU, m⁷g_{3′OMe}pppUpA, m⁷g_{3′OMe}pppUpC, m⁷G_{3′OMe}pppUpG, and m⁷G_{3′OMe}pppUpU.

In some embodiments, a trinucleotide cap comprises m⁷G_{3′OMe}pppApA. In some embodiments, a trinucleotide cap comprises m⁷G_{3′OMe}pppApC. In some embodiments, a trinucleotide cap comprises m⁷G_{3′OMe}pppApG. In some embodiments, a trinucleotide cap comprises m⁷G_{3′OMe}pppApU. In some embodiments, a trinucleotide cap comprises m⁷G_{3′OMe}pppCpA. In some embodiments, a trinucleotide cap comprises m⁷ G_{3′OMe}pppCpC. In some embodiments, a trinucleotide cap comprises m⁷G_{3′OMe}pppCpG. In some embodiments, a trinucleotide cap comprises m²G_{3′OMe}pppCpU. In some embodiments, a trinucleotide cap comprises m⁷G_{3′OMe}pppGpA. In some embodiments, a trinucleotide cap comprises m⁷G_{3′OMe}pppGpC. In some embodiments, a trinucleotide cap comprises m⁷G_{3′OMe}pppGpG. In some embodiments, a trinucleotide cap comprises m⁷G_{3′OMe}pppGpU. In some embodiments, a trinucleotide cap comprises m⁷G_{3′OMe}pppUpA. In some embodiments, a trinucleotide cap comprises m⁷G_{3′OMe}pppUpC. In some embodiments, a trinucleotide cap comprises m⁷G_{3′OMe}pppUpG. In some embodiments, a trinucleotide cap comprises m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}PC, m⁷G_{3′OMe}pppA_{2′OMe}pG, m⁷G_{3′OMe}pppA_{2′OMe}pU, m⁷G_{3′OMe}pppC_{2′OMe}PA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppU_{2′OMe}pA, m⁷G_{3′OMe}pppU_{2′OMe}pC, m⁷G_{3′OMe}pppu_{2′OMe}pG, and m⁷G_{3′OMe}pppU_{2′OMe}pU.

In some embodiments, a trinucleotide cap comprises m⁷G_{3′OMe}pppA_{2′OMe}pA. In some embodiments, a trinucleotide cap comprises m⁷G_{3′OMe}pppA_{2′OMe}pC. In some embodiments, a trinucleotide cap comprises m⁷G_{3′OMe}pppA_{2′OMe}pG. In some embodiments, a trinucleotide cap comprises m⁷G_{3′OMe}pppA_{2′OMe}pU. In some embodiments, a trinucleotide cap comprises m⁷G_{3′OMe}pppC_{2′OMe}pA. In some embodiments, a trinucleotide cap comprises m⁷G_{3′OMe}pppC_{2′OMe}pC. In some embodiments, a trinucleotide cap comprises m⁷G_{3′OMe}pppC_{2′OMe}pG. In some embodiments, a trinucleotide cap comprises m⁷G_{3′OMe}pppC_{2′OMe}pU. In some embodiments, a trinucleotide cap comprises m⁷G_{3′OMe}pppG_{2′OMe}pA. In some embodiments, a trinucleotide cap comprises m⁷G_{3′OMe}pppG_{2′OMe}PC. In some embodiments, a trinucleotide cap comprises m⁷G_{3′OMe}pppG_{2′OMe}pG. In some embodiments, a trinucleotide cap comprises m⁷G_{3′OMe}pppG_{2′OMe}pU. In some embodiments, a trinucleotide cap comprises m⁷G_{3′OMe}pppU_{2′OMe}pA. In some embodiments, a trinucleotide cap comprises m⁷G_{3′OMe}pppU_{2′OMe}pC. In some embodiments, a trinucleotide cap comprises m⁷G_{3′OMe}pppU_{2′OMe}pG. In some embodiments, a trinucleotide cap comprises m⁷G_{3′OMe}pppU_{2′OMe}pU.

A trinucleotide cap, in still other embodiments, comprises a sequence selected from the following sequences: m⁷Gpppa_{2′OMe}pA, m⁷Gpppa_{2′OMe}pC, m⁷Gpppa_{2′OMe}PG, m⁷Gpppa_{2′OMe}pU, m⁷Gpppc_{2′OMe}pA, m⁷Gpppo_{2′OMe}pC, m⁷Gpppc_{2′OMe}pG, m⁷Gpppc_{2′OMe}pU, m⁷Gpppg_{2′OMe}pA, m⁷Gpppg_{2′OMep}C, m⁷Gpppg_{3′OMe}pG, m⁷Gpppg_{3′OMe}pU, m⁷Gpppu_{2′OMe}pA, m⁷Gpppu_{2′OMe}pC, m⁷GpppU_{2′OMe}pG, and m⁷GpppU_{2′OMe}pU.

In some embodiments, a trinucleotide cap comprises m⁷GpppA_{2′OMe}pA. In some embodiments, a trinucleotide cap comprises m⁷GpppA_{2′OMe}pC. In some embodiments, a trinucleotide cap comprises m⁷GpppA_{2′OMe}pG. In some embodiments, a trinucleotide cap comprises m⁷GpppA_{2′OMe}pU. In some embodiments, a trinucleotide cap comprises m⁷GpppC_{2′OMe}pA. In some embodiments, a trinucleotide cap comprises m⁷GpppC_{2′OMe}pC. In some embodiments, a trinucleotide cap comprises m⁷GpppC_{2′OMe}pG. In some embodiments, a trinucleotide cap comprises m⁷GpppC_{2′OMe}pU. In some embodiments, a trinucleotide cap comprises m⁷GpppG_{2′OMe}pA. In some embodiments, a trinucleotide cap comprises m⁷GpppG_{2′OMe}pC. In some embodiments, a trinucleotide cap comprises m⁷GpppG_{2′OMe}pG. In some embodiments, a trinucleotide cap comprises m⁷GpppG_{2′OMe}pU. In some embodiments, a trinucleotide cap comprises m⁷GpppU_{2′OMe}pA. In some embodiments, a trinucleotide cap comprises m⁷GpppU_{2′OMe}pC. In some embodiments, a trinucleotide cap comprises m⁷GpppU_{2′OMe}pG. In some embodiments, a trinucleotide cap comprises m⁷GpppU_{2′OMe}pU.

In some embodiments, a trinucleotide cap comprises m⁷Gpppm⁶A_{2′OMe}pG. In some embodiments, a trinucleotide cap comprises m⁷Gpppc⁶A_{2′OMe}pG.

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

In some embodiments, a trinucleotide cap comprises any one of the following structures:

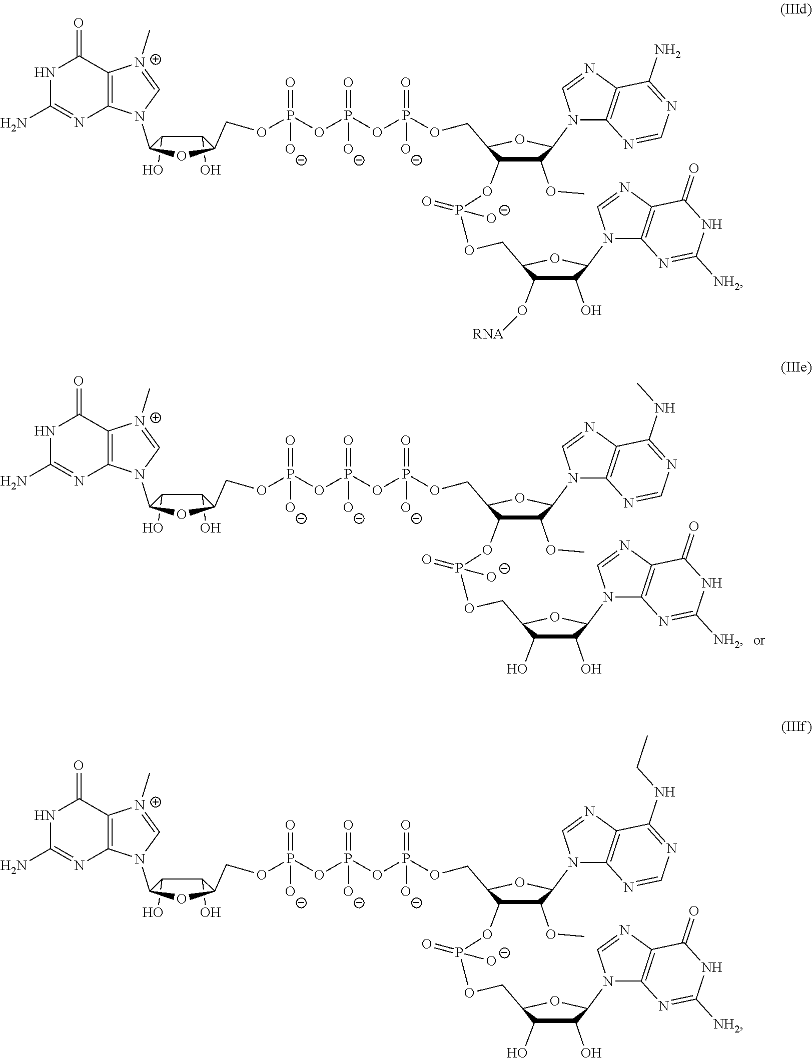

or a stereoisomer, tautomer, or salt thereof.

In some embodiments, the cap analog comprises a tetranucleotide cap. In some embodiments, the tetranucleotide cap comprises a trinucleotide as set forth above. In some embodiments, the tetranucleotide cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

### Formula (IV)

As described herein, in some embodiments, the tetranucleotide cap comprises formula (IV):

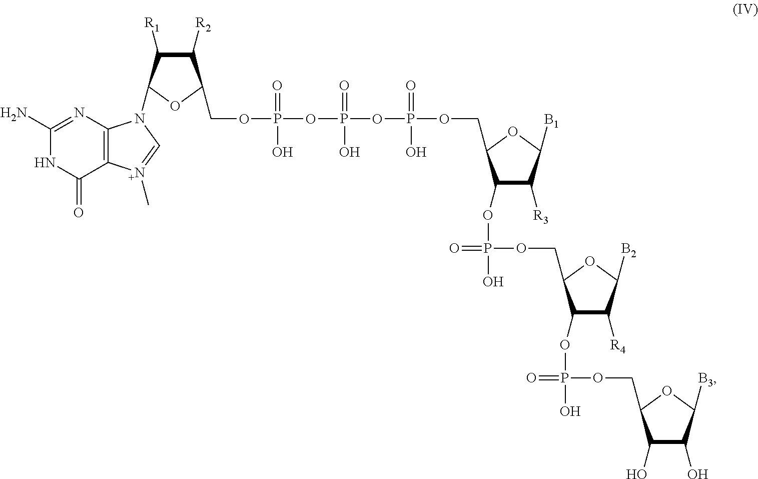

or a stereoisomer, tautomer, or salt thereof,
wherein B₁, B₂, and B₃ are independently a natural, a modified, or an unnatural nucleoside based; and R¹, R², R³, and R⁴ are independently OH or O-methyl. In some embodiments, R³ is O-methyl and R⁴ is OH. In some embodiments. R³ and R⁴ are O-methyl. In some embodiments. R⁴ is O-methyl. In some embodiments, R; is OH, R² is OH, R³ is O-methyl, and R⁴ is OH. In some embodiments, R₁ is OH, R² is OH, R³ is O-methyl, and R⁴ is O-methyl. In some embodiments, at least one of R¹ and R² is O-methyl, R³ is O-methyl, and R⁴ is OH. In some embodiments, at least one of R¹ and R² is O-methyl, R³ is O-methyl, and R⁴ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N₆-methyladenine. In some embodiments, B₁ is adenine, cytosine, thymine, or uracil. In some embodiments, B₁ is adenine, B₂ is uracil, and B₃ is adenine. In some embodiments, R¹ and R² are OH, R³ and R⁴ are O-methyl, B₁ is adenine, B₂ is uracil, and B₃ is adenine.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A tetranucleotide cap, in some embodiments, comprises a sequence selected from the following sequences: m⁷G_{3′OMe}pppApApN, m²G_{3′OMe}pppApCpN, m⁷G_{3′OMe}pppApGpN, m²G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m¹G_{3′OMe}pppGpApN, m¹G_{3′OMe}pppOpCpN, m²G_{3′OMe}pppGpGpN, m⁷G_{3′OMe}pppGpUpN, m⁷G_{3′OMe}pppUpApN. m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A tetranucleotide cap, in other embodiments, comprises a sequence selected from the following sequences: m⁷G_{3′OMe}pppA_{2′OMe}papN, m⁷G_{3′OMe}pppA_{2′OMe}pcpN, m⁷G_{3′OMe}pppA_{2′OMe}pgpN, m⁷G_{3′OMe}pppA_{2′OMe}pupN, m⁷G_{3′OMe}pppC_{2′OMe}papN, m⁷G_{3′OMe}pppC_{2′OMe}pcpN, m⁷G_{3′OMe}pppC_{2′OMe}pgpN, m⁷G_{3′OMe}pppC_{2′OMe}pupN, m⁷G_{3′OMe}pppG_{3′OMe}papN, m⁷G_{2′OMe}pppG_{3′OMe}pcpN, m⁷G_{3′OMe}pppG_{3′OMe}pgpN, m⁷G_{3′OMe}pppG_{3′OMe}pupN, m⁷G_{3′OMe}pppU_{2′OMe}papN, m⁷G_{3′OMe}pppU_{3′OMe}pcpN, m⁷G_{3′OMe}pppU_{2′OMe}pGpN, and m⁷G_{3′OMe}pppU_{2′OMe}pUpN, where N is a natural, a modified, or an unnatural nucleoside base.

A tetranucleotide cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m¹GpppC_{2′OMe}pUpN, m⁷GpppG_{3′OMe}pApN. m⁷GpppG_{3′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A tetranucleotide cap, in other embodiments, comprises a sequence selected from the following sequences: m⁷g_{3′OMe}ppp A_{2′OMe}pA_{2′OMe}pN, m g_{3′OMe}pppA_{2′OMe}pC_{2′OMe}pN, m⁷g_{3′OMe}pppA_{2′OMe}pG_{3′OMe}pN, m⁷g_{3′OMe}pppA_{2′OMe}pU_{2′OMe}pN. m⁷g_{3′OMe}pppC_{2′OMe}PA_{2′OMe}pN, m⁷g_{3′OMe}pppC_{2′OMe}pC_{2′OMe}pN, m⁷g_{3′OMe}pppC_{2′OMe}pG_{3′OMe}pN, m⁷g_{3′OMe}pppC_{2′OMe}pU_{2′OMe}pN, m⁷g_{3′OMe}pppG_{3′OMe}pA_{2′OMe}pN, m⁷g_{3′OMe}pppG_{2′OMe}pC_{2′OMe}pN, m⁷g_{3′OMe}pppG_{2′OMe}pG_{3′OMe}pN, m⁷g_{3′OMe}pppU_{2′OMe}pU_{2′OMe}pN, m⁷g_{3′OMe}ppp U_{2′OMe}pA_{2′OMe}pN, m⁷g_{3′OMe}pppU_{2′OMe}pC_{2′OMe}pN, m⁷g_{3′OMe}pppU_{2′OMe}pg_{2′OMe}pN, and m g_{3′OMe}pppU_{2′OMe}pU_{2′OMe}pN, where N is a natural, a modified, or an unnatural nucleoside base.

A tetranucleotide cap, in still other embodiments, comprises a sequence selected from the following sequences: m⁷GpppA_{2′OMe}pa_{2′OMe}pn, m⁷GpppA_{2′OMe}Pc_{2′OMe}pn, m⁷GpppA_{2′OMe}pg_{2′OMe}pn, m⁷GpppA_{2′OMe}pu_{2′OMe}pn, m⁷GpppC_{2′OMe}pa_{2′OMe}pn, m⁷GpppC_{2′OMe}pc_{2′OMe}pn, m⁷GpppC_{2′OMe}pg_{3′OMe}pn, m⁷GpppC_{2′OMe}pu_{2′OMe}pn, m⁷GpppG_{2′OMe}pa_{2′OMe}pn, m⁷GpppG_{2′OMe}pC_{2′OMe}pn, m⁷GpppG_{2′OMe}pg_{2′OMe}pn, m⁷GpppG_{2′OMe}pu_{2′OMe}pn, m⁷GpppU_{2′OMe}pa_{2′OMe}pn, m⁷GpppU_{2′OMe}pC_{2′OMe}pn, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a tetranucleotide cap comprises GGAG. In some embodiments, a tetranucleotide cap comprises the following structure:

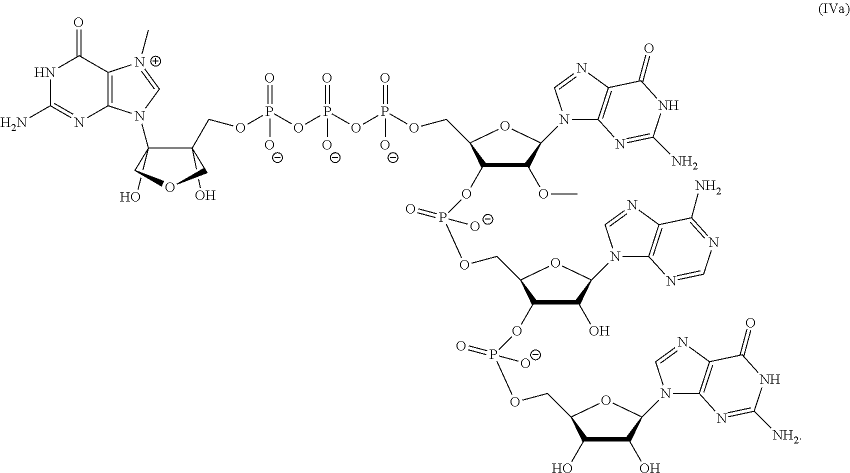

The capping efficiency of a post-transcriptional or co-transcriptional capping reaction may vary. As used herein “capping efficiency” refers to the amount (e.g., expressed as a percentage) of mRNAs comprising a cap structure relative to the total mRNAs in a mixture (e.g., a post-translational capping reaction or a co-transcriptional calling reaction). In some embodiments, the capping efficiency of a capping reaction is at least 60%, 70%, 80%, 90%, 95%, 99%, or 99.9% (e.g., after the capping reaction at least 60%, 70%, 80%, 90%, 95%, 99%, or 99.9% of the input mRNAs comprise a cap). In some embodiments, multivalent co-IVT reactions described herein do not affect the capping efficiency of the mRNAs resulting from the IVT reaction.

A 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition comprises an RNA (e.g., mRNA) having an ORF that encodes a signal peptide fused to the expressed polypeptide. Signal peptides, usually comprising the N-terminal 15-60 amino acids of proteins, are typically needed for the translocation across the membrane on the secretory pathway and, thus, universally control the entry of most proteins both in eukaryotes and prokaryotes to the secretory pathway. A signal peptide may have a length of 15-60 amino acids.

In some embodiments, an ORF encoding a polypeptide is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias % G/C content to increase mRNA thermodynamic stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, an RNA (e.g., mRNA) is not chemically modified and comprises the standard ribonucleotides consisting of adenosine, guanosine, cytosine and uridine. In some embodiments, nucleotides and nucleosides comprise standard nucleoside residues such as those present in transcribed RNA (e.g. A, G, C, or U). In some embodiments, nucleotides and nucleosides comprise standard deoxyribonucleosides such as those present in DNA (e.g. dA, dG, dC, or dT).

The compositions can comprise, in some embodiments, an RNA having an open reading frame encoding a polypeptide, wherein the nucleic acid comprises nucleotides and/or nucleosides that can be standard (unmodified) or modified as is known in the art. In some embodiments, nucleotides and nucleosides comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide is one as is generally known or recognized in the art. Non-limiting examples of such naturally occurring modified nucleotides and nucleotides can be found, inter alia, in the widely recognized MODOMICS database.

Also provided are modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

In some embodiments, modified nucleosides in nucleic acids (e.g., RNA nucleic acids, such as mRNA nucleic acids) comprise N1-methyl-pseudouridine (m1ψ), 1-ethyl-pseudouridine (c1ψ), 5-methoxy-uridine (mo5U), 5-methyl-cytidine (m⁵C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxycytidine. In some embodiments, the polyribonucleotide includes a combination of at least two (e.g., 2, 3, 4 or more) of any of the aforementioned modified nucleobases, including but not limited to chemical modifications.

In some embodiments, an mRNA comprises N1-methyl-pseudouridine (m1ψ) substitutions at one or more or all uridine positions of the nucleic acid.

In some embodiments, an mRNA comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA comprises pseudouridine (ψ) substitutions at one or more or all uridine positions of the nucleic acid.

In some embodiments, a mRNA pseudouridine (ψ) substitutions at one or more or all uridine positions of the nucleic acid and 5-methyl cytidine substitutions at one or more or all cytidine positions of the nucleic acid.

In some embodiments, a mRNA comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids may be partially or fully modified along the entire length of the molecule. For example, one or more or all or a given type of nucleotide (e.g., purine or pyrimidine, or any one or more or all of A, G, U, C) may be uniformly modified in a nucleic acid, or in a predetermined sequence region thereof (e.g., in the mRNA including or excluding the poly(A) tail). In some embodiments, all nucleotides X in a nucleic acid (or in a sequence region thereof) are modified nucleotides, wherein X may be any one of nucleotides A, G. U, C, or any one of the combinations A+G, A+U, A+C, G+U, G+C, U+C, A+G+U, A+G+C, G+U+C or A+G+C.

The mRNAs may comprise one or more regions or parts which act or function as an untranslated region. Where mRNAs are designed to encode at least one polypeptide of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. The regulatory features of a UTR can be incorporated into the polynucleotides to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more proteins. In some embodiments, the UTR is heterologous to the ORF encoding the one or more proteins. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids as flanking regions to an ORF. This publication is incorporated by reference herein for this purpose.

Additional exemplary UTRs that may be utilized in the nucleic acids include, but are not limited to, one or more 5′ UTRs and/or 3′ UTRs derived from the nucleic acid sequence of: a globin, such as an α-or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B₄ (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Largo 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA)₅′ UTR; a hydroxysteroid (17-β) dehydrogenase (HSD17B₄)₅′ UTR; a Tobacco etch virus (TEV)₅′ UTR; a Venezuelen equine encephalitis virus (TEEV)₅′ UTR; a 5′ proximal open reading frame of rubella virus (RV) RNA encoding nonstructural proteins; a Dengue virus (DEN)₅′ UTR; a heat shock protein 70 (Hsp70)₅′ UTR; a elF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH)₃′ UTR; a VEEV 3′ UTR; a hepatitis B virus (HBV)₃′ UTR; α-globin 3′ UTR; a DEN 3′ UTR; a PAV barley yellow dwarf virus (BYDV-PAV)₃′ UTR; an elongation factor 1 α1 (EEF1A1)₃′ UTR; a manganese superoxide dismutase (MnSOD)₃′ UTR; a β subunit of mitochondrial H (+)-ATP synthase (8-mRNA)₃′ UTR; a GLUT1 3′ UTR; a MEF2A 3′ UTR; a β—FI-ATPase 3′ UTR; functional fragments thereof and combinations thereof.

Wild-type UTRs derived from any gene or mRNA can be incorporated into the nucleic acids. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8 (3): 568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, e.g., US 2010/0129877, the contents of which are incorporated herein by reference for this purpose).

The nucleic acids can comprise combinations of features. For example, the ORF can be flanked by a 5′ UTR that comprises a strong Kozak translational initiation signal and/or a 3′ UTR comprising an oligo (dT) sequence for templated addition of a polyA tail. A 5′ UTR can comprise a first nucleic acid fragment and a second nucleic acid fragment from the same and/or different UTRs (see, e.g., US 2010/0293625, herein incorporated by reference in its entirety for this purpose).

Other non-UTR sequences can be used as regions or subregions within the nucleic acids. For example, introns or portions of intron sequences can be incorporated into the nucleic acids. Incorporation of intronic sequences can increase protein production as well as nucleic acid expression levels. In some embodiments, the nucleic acid comprises an internal ribosome entry site (IRES) instead of or in addition to a UTR (see, e.g., Yakuboy et al., Biochem. Biophys Res Commun. 2010. 394 (1): 189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Tails

Some aspects relate to methods of producing RNAs containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, “polyA-tailing efficiency” refers to the amount (e.g., expressed as a percentage) of mRNAs having polyA tail that are produced by an IVT reaction using an input DNA relative to the total number of mRNAs produced in the IVT reaction using the input DNA. The poly A-tailing efficiency of an IVT reaction may vary, for example depending upon the RNA polymerase used, amount or purity of input DNA used, etc. In some embodiments, the poly A-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography).

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a poly A tail. The efficiency (e.g., percentage of polyA tail-containing RNAs in an RNA composition may be measured i) after the IVT reaction and before purification, or ii) after the RNA composition has been purified (e.g., by chromatography, such as oligo-dT chromatography).

Unique polyA tail lengths provide certain advantages to nucleic acids. Generally, the length of a poly A tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the poly A tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The poly A tail can also be designed as a fraction of the nucleic acid to which it belongs. In this context, the poly A tail can be 10, 20, 30, 40, 50, 60, 70, 80, or 90% or more of the total length of the construct, a construct region, or the total length of the construct minus the poly A tail. Further, engineered binding sites and conjugation of nucleic acids for PolyA-binding protein can enhance expression.

### In Vitro Transcription

Some aspects relate to mRNAs produced by “in vitro transcription” or IVT. IVT methods produce (e.g., synthesize) an RNA transcript (e.g., mRNA transcript) by contacting a DNA template (e.g., a first input DNA and a second input DNA) with an RNA polymerase (e.g., a T7 RNA polymerase, a T7 RNA polymerase variant, etc.) under conditions that result in the production of the RNA transcript. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography.

### Multivalent In Vitro Transcription (IVT)

Some aspects relate to multivalent in vitro transcription. Multivalent in vitro transcription refers to contacting two or more DNA templates (e.g., a first input DNA and a second input DNA) with an RNA polymerase (e.g., a T7 RNA polymerase) under conditions that result in the production of RNA transcripts.

Each input DNA (e.g., in a population of input DNA templates) in a co-IVT reaction may be obtained from a different source than other input DNAs. For example, each input DNA may be obtained from a different bacterial cell or population or bacterial cells. For example, in a co-IVT reaction having three populations of input DNAs, a first input DNA can be produced in bacterial cell population A, a second input DNA can be produced in bacterial cell population B, and a third input DNA can be produced in bacterial cell population C, where each of A, B, and C are not the same bacterial culture (e.g., co-cultured in the same container or plate). In another example, different input DNAs are obtained by separate synthesis reactions or produced by separate amplification reactions.

The amounts of input DNAs used in multivalent co-IVT reactions may be normalized. Normalization may be based, for example, on the molar masses, lengths, nucleotide contents, degradation rates, and/or purity of input DNAs. In some embodiments, normalization is based on the degradation rate of resulting RNAs.

Normalization may be based on the lowest level of a certain characteristic present among the input DNAs (e.g., lowest molar mass, degradation rate (e.g., of the input DNA and/or output RNA), nucleotide content, purity, and/or poly A-tailing efficiency). Alternatively, normalization may be based on the highest level of a certain characteristic present among the input DNAs (e.g., highest molar mass, degradation rate (e.g., of the input DNA and/or output RNA), nucleotide context, purity, and/or poly A-tailing efficiency). In some embodiments, normalization is based on the rate of RNA production from the input DNAs (e.g., the highest rate of RNA production of an input DNA or the lowest rate of RNA production of an input DNA in a reaction mixture).

The amount of one or more input DNAs may be adjusted and/or normalized to improve production of RNA compositions having a pre-defined or desired ratio of RNA components. Adjusting and/or normalizing amounts of input DNAs may compensate for differences between input DNAs (e.g., large differences in lengths of two input DNAs, or different polyA tailing efficiencies) that can affect the ratio of RNAs in a multivalent RNA composition, thereby allowing for the production of RNA compositions having desired ratios of different RNAs. For example, the amount of two input DNAs present in a co-IVT reaction may be determined by selecting a desired molar ratio of a first RNA to a second RNA, calculating the mass of each DNA template necessary to achieve the same molar ratio between input DNAs, and combining input DNAs encoding each of the first and second RNAs in the same molar ratio.

The number of input DNAs (e.g., populations of input DNA molecules) used in an IVT reaction may vary, depending upon the number of different RNA molecules desired to be included in the multivalent RNA composition. An IVT reaction mixture may comprise 2 or more different input DNAs (e.g., 2, 3, 4, 5, 6, 7, 8, 9, 10, or more different input DNAs).

The concentration of each of the populations of DNA molecules may also vary.

The input DNAs may be added to an IVT reaction are a predefined DNA ratio, which may comprise a ratio between 2, 3, 4, 5, 6, 7, 8, 9, 10, or more different input DNAs (e.g., depending on the number of different RNAs in a composition).

The size of two or more input DNAs (e.g., DNAs in two or more different populations of input DNAs) may also vary.

The mass of each population of input DNA molecules in an IVT reaction may also vary.

The molar ratio between populations of input DNA molecules in an IVT reaction may also vary.

Different input DNA molecules used in an IVT reaction may have a different length (e.g., comprises a different number of nucleotides).

A co-IVT reaction may include co-transcription of at least 2 different input DNAs (e.g., at least 2 of DNA A, B, C, D, E, F, F, H, I, J, etc.) at a ratio of A:B:C:D:E:F:G:H:I:J, wherein if DNA A is normalized to 1, one or more of DNA B, C, D, E, F, G, H, I, J, etc. can each independently be present at an amount (e.g., a concentration) that is from 0.01 to 100 times the amount (e.g., a concentration) of A. One or more of DNA B, C, D, E, F, G, H, I, or J may also be absent.

A multivalent RNA composition may be produced by combining RNA transcripts (e.g., mRNAs) from separate sources. For example, each of two or more DNA templates may be transcribed in separate IVT reactions, and combined to produce a multivalent RNA composition. RNAs may be combined in any desired amount to produce a multivalent RNA composition comprising two or more RNAs in a specific ratio.

### Identification and Ratio Determination (IDR) Sequences

In some embodiments, one or more nucleic acids comprises an Identification and Ratio Determination sequence. An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR-or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

### Formula (AI)

In some embodiments, the ionizable amino lipid is a compound of Formula (AI):

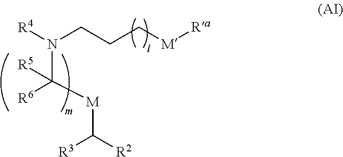

or its N-oxide, or a salt or isomer thereof,
wherein R′^{a} is R′^{branched}, wherein
R′^{branched} is:

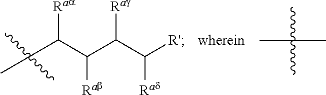

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

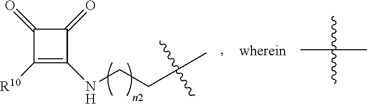

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

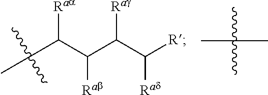

denotes a point of attachment; R_{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched};
R′^{branched} is

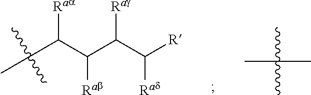

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R⁴ is a C₁₋₁₂ alkyl; 1 is 3; and m is 7.

In some embodiments of the compounds of Formula (AI), R′^{a} is R′^{branched}.
R′^{branched} is

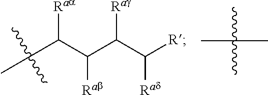

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

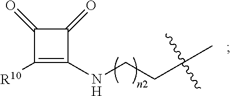

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched};
R′^{branched} is

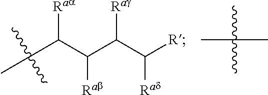

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (AI) is selected from:

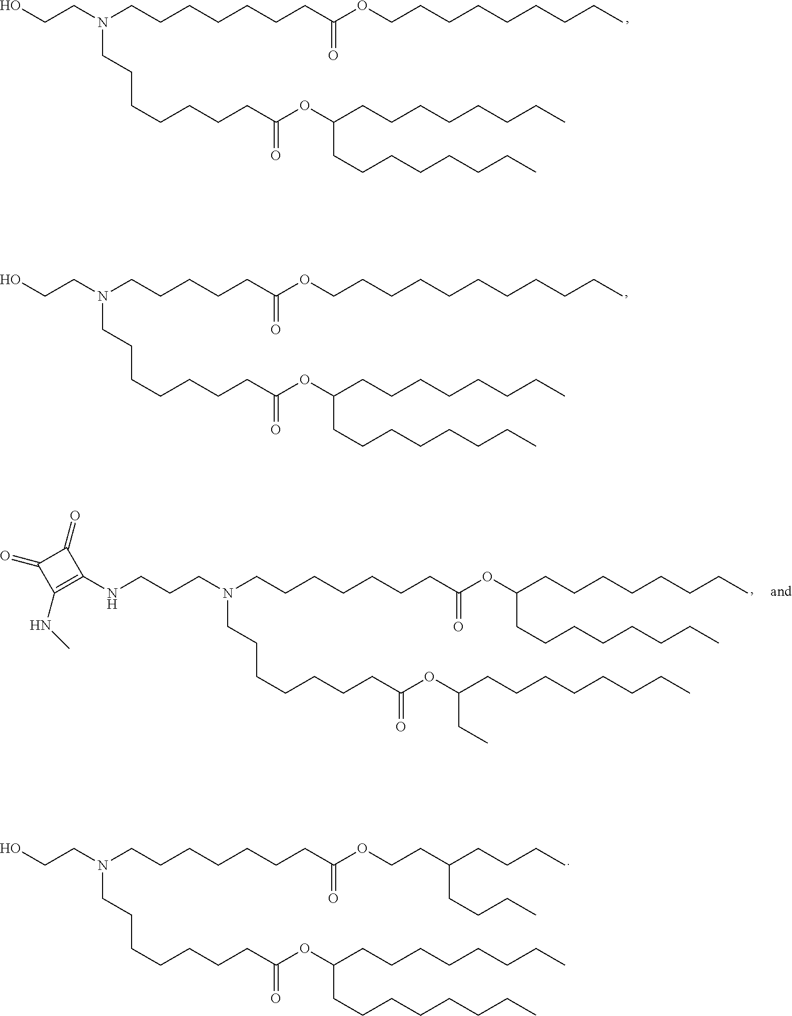 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

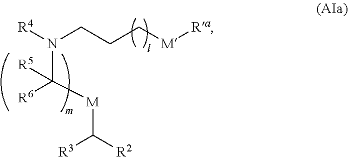

or its N-oxide, or a salt or isomer thereof,
wherein R′^{a} is R′^{branched}, wherein
R′^{branched} is:

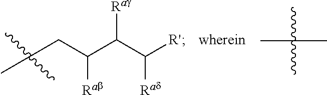

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

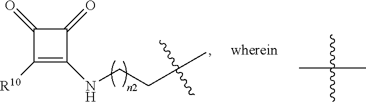

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (Alb):

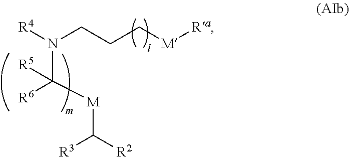

or its N-oxide, or a salt or isomer thereof,
wherein R′^{a} is R′^{branched}, wherein
R′^{branched} is:

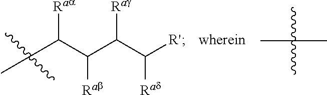

denotes a point of attachment;
wherein R^{aα}, R^{aβ}, R^{aγ}, and R^{aδ} are each independently selected from the group consisting of H. C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some embodiments of Formula (AI) or (Alb), R′^{a} is R′^{branched}, R′^{branched} is

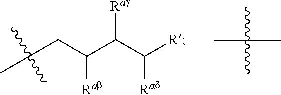

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R^{e} is H; M and M′ are each —C(O)O—; R′ is a C₁₋₁₂ alkyl; 1 is 5; and m is 7.

In some embodiments of Formula (AI) or (AIb), R′^{a} is R′^{branched}, R′_{branched} is

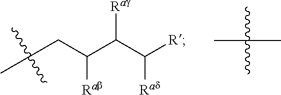

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}. R′^{branched}; is

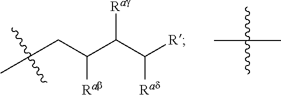

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid of Formula (AI) is a compound of Formula (AIc):

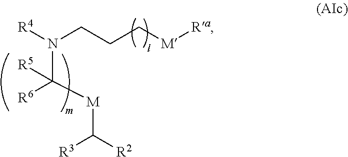

or its N-oxide, or a salt or isomer thereof,
wherein R′^{a} is R′^{branched}, wherein
R′^{branched} is:

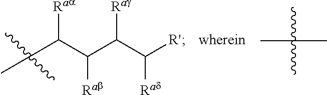

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;

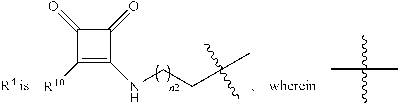

denotes a point of attachment; whereinR¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

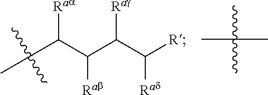

denotes a point of attachment; R^{aβ}, R^{aγ}, and Ras are each H; R^{aα} is C₂₋₁₂ alkyl; R² and R³ are each C₁₋₁₄ alkyl; R⁴ is

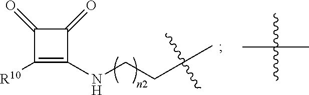

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

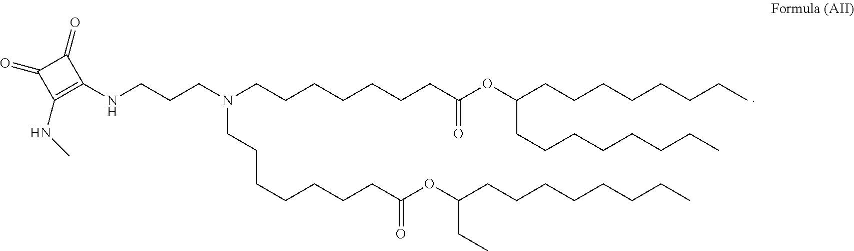 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

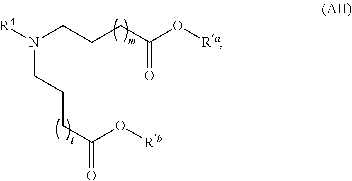

or its N-oxide, or a salt or isomer thereof,
wherein R′^{a} is R′^{branched} or R′^{cyclic}, wherein
R′^{branched} is:

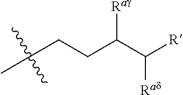

and R′^{cyclic} is:

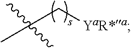

and
R′^{b} is:

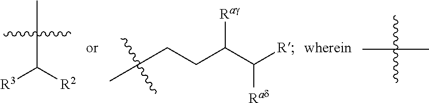

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

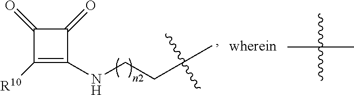

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{α} is a C₃₋₆ carbocycle;
R*″^{α} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

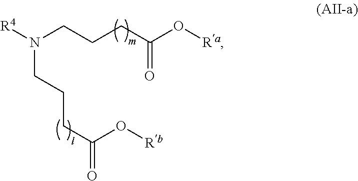

or its N-oxide, or a salt or isomer thereof,
wherein R′^{a} is R′^{branched} or R′^{cyclic}, wherein

R′^{branched} is:

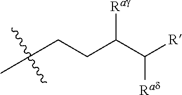

and R′^{b} is:

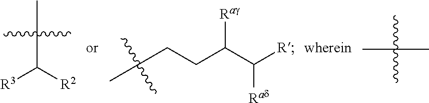

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

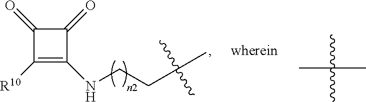

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

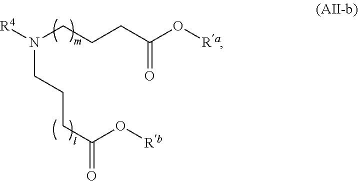

or its N-oxide, or a salt or isomer thereof,
wherein R′^{a} is R′^{branched} or R′^{cyclic}; wherein
R′^{branched} is:

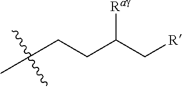

and R′^{b} is:

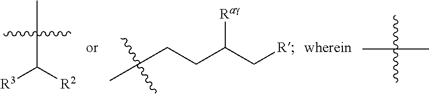

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

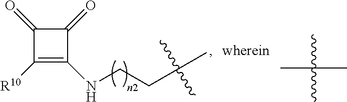

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

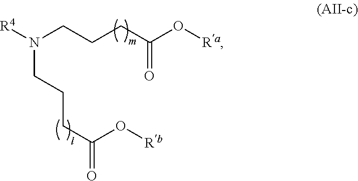

or its N-oxide, or a salt or isomer thereof,
wherein R′^{a} is R′^{branched} or R′^{cyclic}; wherein
R′^{branched} is:

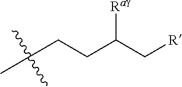

### and R′^{b} is:

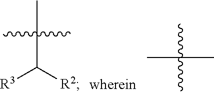

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

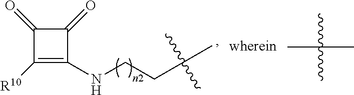

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

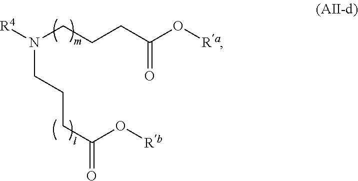

or its N-oxide, or a salt or isomer thereof,
wherein R′^{a} is R′^{branched} or R′^{cyclic}, wherein
R′^{branched} is:

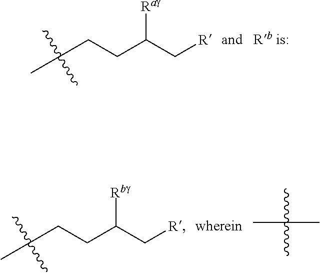

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

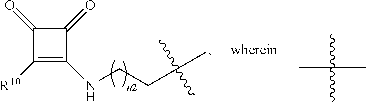

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

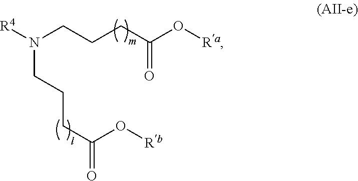

or its N-oxide, or a salt or isomer thereof,
wherein R′^{a} is R′^{branched} or R′^{cyclic}, wherein
R′^{branched} is:

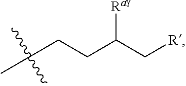

### and R′^{b} is:

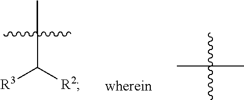

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

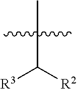

and R² and R³ are each independently a C₁₋₁₄ alkyl. In some embodiments of the compound of Formula (AII), (AII-a), (AII-b), (AII-c), (AII-d), or (AII-e), R′^{b} is:

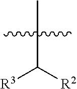

and R² and R³ are each independently a C₆₋₁₀ alkyl. In some embodiments of the compound of Formula (AII), (AII-a), (AII-b), (AII-c), (AII-d), or (AII-c), R′^{b} is:

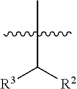

and R² and R³ are each a C₈ alkyl.

In some embodiments of the compound of Formula (AII), (AII-a), (AII-b), (AII-c), (AII-d), or (AII-e), R′^{branched} is:

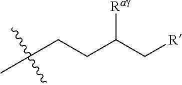

### and R′^{b} is:

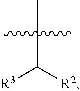

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

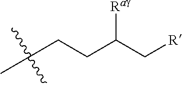

### and R′^{b} is:

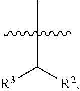

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

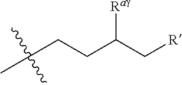

### and R′^{b} is:

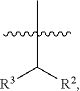

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

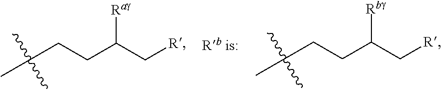

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

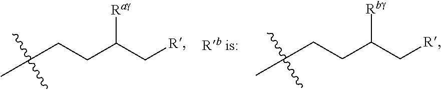

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

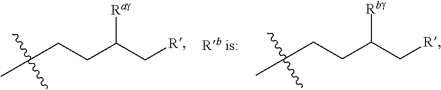

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

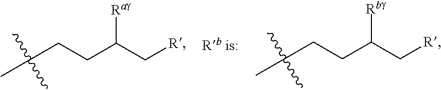

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

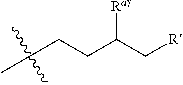

### and R′^{b} is:

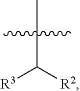

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

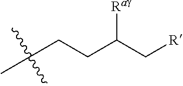

### and R′^{b} is:

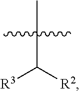

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

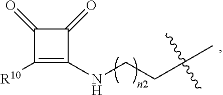

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

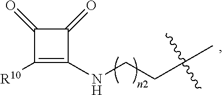

wherein R¹⁰ is NH(CH₃) and n2 is 2.

In some embodiments of the compound of Formula (AII), (AII-a), (AII-b), (AII-c), (AII-d), or (AII-e), R′^{branched} is:

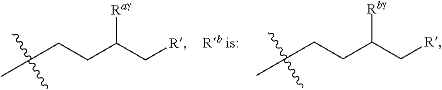

m and l are each independently selected from 4, 5, and 6, each R′ independently is a C₁₋₁₂ alkyl, R^{aγ} and R^{bγ} are each a C₁₋₁₂ alkyl, and R⁴ is

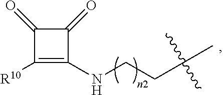

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

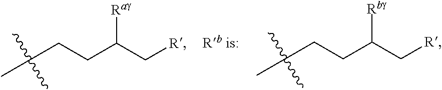

m and l are each 5, each R′ independently is a C₂₋₅ alkyl, R^{aγ} and REY are each a C₂₋₆ alkyl, and R⁴ is

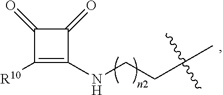

wherein R¹⁰ is NH(CH₃) and n2 is 2.

In some embodiments of the compound of Formula (AII), (AII-a), (AII-b), (AII-c), (AII-d), or (AII-e), R′^{branched} is:

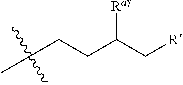

### and R′^{b} is:

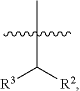

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

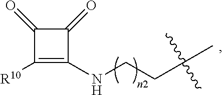

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

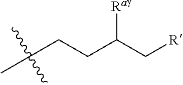

### and R′^{b} is:

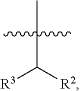

m and l are each 5, R′ is a C₂₋₅ alkyl, R^{aγ} is a C₂₋₆ alkyl, R² and R³ are each a C₈ alkyl, and R⁴ is

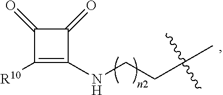

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

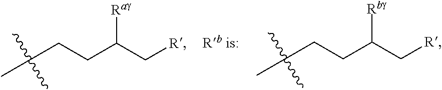

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

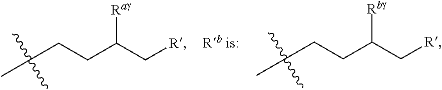

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

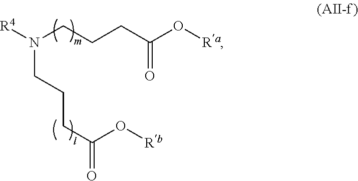

or its N-oxide, or a salt or isomer thereof,
wherein R′^{a} is R′^{branched} or R′^{cyclic}; wherein
R′^{branched} is:

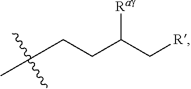

and R′^{b} is:

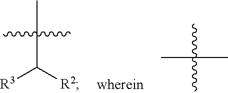

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (All-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

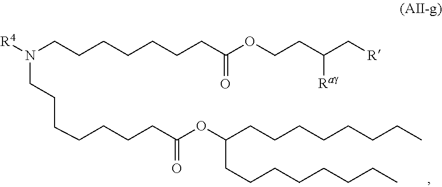

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

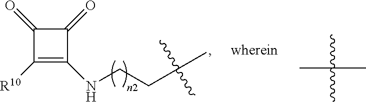

denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

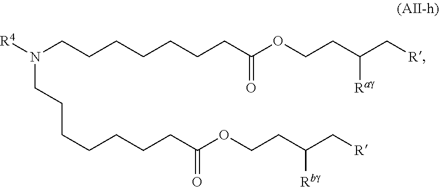

or its N-oxide, or a salt or isomer thereof; wherein
R^{aγ} and REY are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

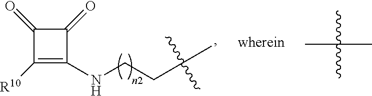

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

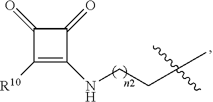

wherein
R¹⁰ is NH(CH₃) and n2 is 2.

In some embodiments of the compound of Formula (AII-g) or (AII-h), R⁴ is —(CH₂)₂OH.

### Formula (AIII)

In some embodiments, the ionizable amino lipids may be one or more of compounds of Formula (AIII):

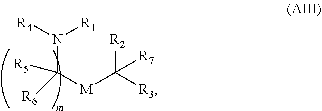

or their N-oxides, or salts or isomers thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R) R 8, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″—C(O)O—, —C(O)N(R′)—,
—N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R₅, together with the atom to which they are attached, form a heterocycle or carbocycle;
R₄ is selected from the group consisting of a C₃₋₆ carbocycle, —(CH₂)ₙQ, —(CH₂)ₙCHQR,
   —CHQR, —CQ(R)₂, and unsubstituted C₁₋₆ alkyl, where Q is selected from a C₃₋₆ carbocycle, a 5-to 14-membered heteroaryl having one or more heteroatoms selected from N, O, and S, —OR,
   —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)O R, and a 5-to 14-membered heterocycloalkyl having one or more heteroatoms selected from N, O, and S which is substituted with one or more substituents selected from oxo (═O), OH, amino, mono-or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkonyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (AIII) includes those in which:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R₅, together with the atom to which they are attached, form a heterocycle or carbocycle;
R₄ is selected from the group consisting of a C₃₋₆ carbocycle, —(CH₂)ₙQ, —(CH₂)ₙCHQR,
   —CHQR, —CQ(R)₂, and unsubstituted C₁₋₆ alkyl, where Q is selected from a C₃₋₆ carbocycle, a 5-to 14-membered heterocycle having one or more heteroatoms selected from N, O, and S, —OR,
   —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR,
—N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR) S(O)₂R, —N(OR)C(O)OR, —N(OR)C(O)N(R)₂, —N(OR) C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5-to 14-membered heterocycle and (i) R₄ is —(CH₂)ₙQ in which n is 1 or 2, or (ii) R₄ is —(CH₂)ₙCHQR in which n is 1, or (iii) R₄ is —CHOR, and —CQ(R)₂, then Q is either a 5-to 14-membered heteroaryl or 8-to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (AIII) includes those in which:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of a C₃₋₆ carbocycle, —(CH₂)ₙQ, —(CH₂)ₙCHOR,
   —CHQR, —CQ(R)₂, and unsubstituted C₁₋₆ alkyl, where Q is selected from a C₃₋₆ carbocycle, a 5-to 14-membered heteroaryl having one or more heteroatoms selected from N, O, and S, —OR
   —O(CH₂), N(R)₂, —C(O)OR, —OC(O)R, —CX₃, —CX₂H, —CXH₂, —CN, —C(O)N(R)₂, —N(R)C(O)R, —N(R) S(O)₂R, —N(R)C(O)N(R)₂, —N(R)C(S)N(R)₂, —CRN(R)₂C(O)OR, —N(R) R₈, —O(CH₂) OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H,
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkonyl, and H:
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (AIII) includes those in which

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R⁴ is —(CH₂)ₙQ or —(CH₂)ₙCHOR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkonyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, another subset of compounds of Formula (AIII) includes those in which
R¹ is selected from the group consisting of C₅₋₃₀ alkyl, C₅₋₂₀ alkonyl, —R*YR″, —YR″, and —R″M′R″;
R² and R₅ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H; each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In certain embodiments, a subset of compounds of Formula (AIII) includes those of Formula (AIII-A):

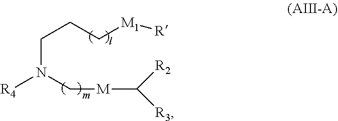

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is
   —OH, —NHC(S)N(R)₂, —NHC(O)N(R)₂, —N(R)C(O)R, —N(R) S(O)₂R, —N(R) R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
   from —C(O)O—, —OC(O)—, —OC(O)-M″—C(O)O—, —C(O)N(R′)—, —P(O)(OR′)O—, —S—S—, an aryl group, and a heteroaryl group,; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-B):

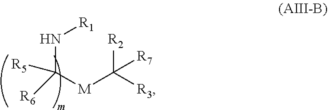

or its N-oxide, or a salt or isomer thereof in which all variables are as defined herein. For example, m is selected from 5, 6, 7, 8, and 9; R⁴ is hydrogen, unsubstituted C₁₋₃ alkyl, or —(CH₂)ₙQ, in which Q is
H, —NHC(S)N(R)₂, —NHC(O)N(R)₂, —N(R)C(O)R, —N(R) S(O)₂R, —N(R) R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″—C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-C):

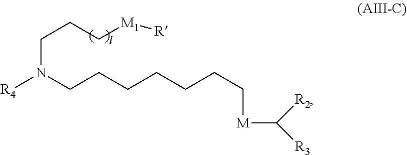

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R) R₈, —NHC(═NR₉)N(R)₂, —NHC(—CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″—C(O)O—, —C(O)N(R′)—, —P(O)(OR′)O—, —S—S—, an aryl group, and a heteroaryl group; and R² and R₅ are independently selected from the group consisting of H, C₁₋₁₄ alkyl, and C₂₋₁₄ alkenyl.

In some embodiments, the compounds of Formula (AIII) are of Formula (AIII-D),

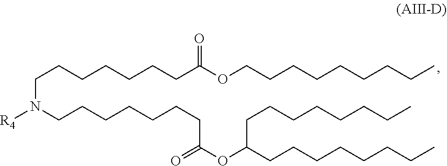

or their N-oxides, or salts or isomers thereof, wherein R⁴ is as described herein.

In another embodiment, the compounds of Formula (AIII) are of Formula (AIII-E),

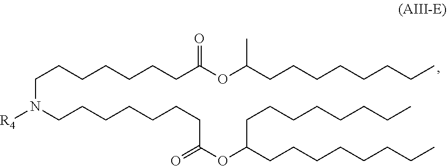

or their N-oxides, or salts or isomers thereof, wherein R⁴ is as described herein.

In another embodiment, the compounds of Formula (AIII) are of Formula (AIII-F) or (AIII-G):

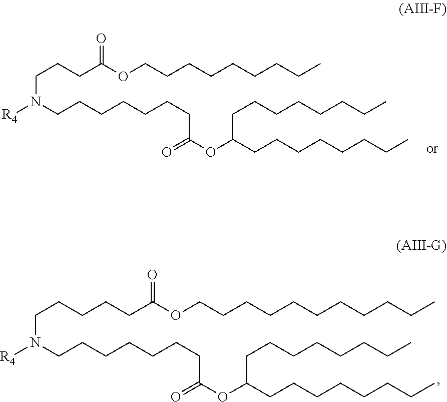

or their N-oxides, or salts or isomers thereof, wherein R⁴ is as described herein.

In another embodiment, the compounds of Formula (AIII) are of Formula (AIII-H):

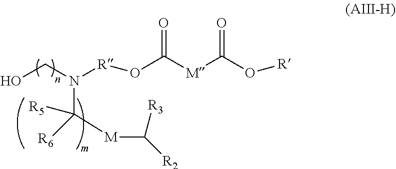

or their N-oxides, or salts or isomers thereof,
wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (AIII) are of Formula (AIII-I):

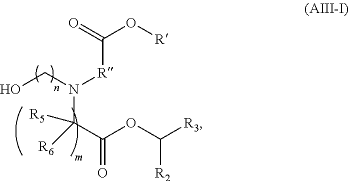

or their N-oxides, or salts or isomers thereof, wherein n is 2, 3, or 4; and m, R′, R″, and R² through R₆ are as described herein. For example, each of R² and R³ may be independently selected from the group consisting of C₅₋₁₄ alkyl and C₅₋₁₄ alkenyl.

In some embodiments, an ionizable amino lipid comprises a compound having structure:

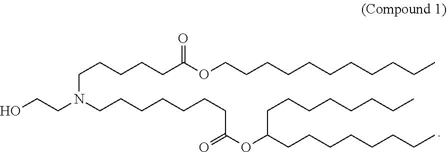 [SMILES: CCCCCCCCCCCOC(=O)CCCCCN(CCO)CCCCCCCC(=O)OC(CCCCCCCC)CCCCCCCC]

In some embodiments, an ionizable amino lipid comprises a compound having structure:

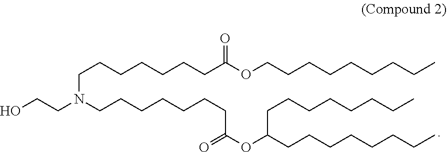 [SMILES: CCCCCCCCCOC(=O)CCCCCCCN(CCO)CCCCCCCC(=O)OC(CCCCCCCC)CCCCCCCC]

In a further embodiment, the compounds of Formula (AIII) are of Formula (AIII-J),

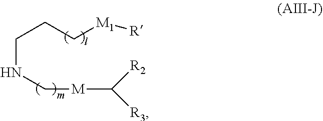

or their N-oxides, or salts or isomers thereof, wherein l is selected from 1, 2, 3, 4, and 5; m is selected from 5, 6, 7, 8, and 9; M₁ is a bond or M′; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R² and R₅ are independently selected from the group consisting of H, C₁₋₁₄ alkyl, and C₂₋₁₄ alkenyl. For example, M″ is C₁₋₆ alkyl (e.g., C₁-4 alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (AIII), (AIII-A), (AIII-B), (AIII-C), (AIII-D). (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids may be one or more of compounds of formula (AIV),

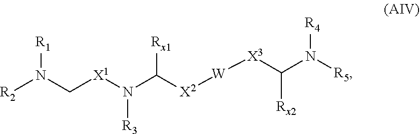

or salts or isomers thereof, wherein

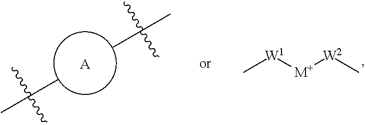

W is

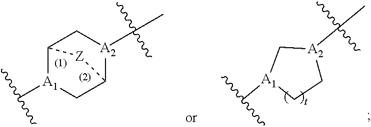

ring A is
t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R¹, R², R³, R⁴, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting

### of —C(O)O—, —OC(O)—, —OC(O)O—, —C(O)N(R′)—, —N(R′) C(O)—, —C(O)—, —C(S)—, —C(S)S—, —SC(S)

—CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, CH₂—, (CH₂)₂—, —CHR—, —CHY—, —C(O)—, —C(O)O—, —OC(O)—, —(CH₂)ₙ—C(O)—, —C(O)—(CH₂)ₙ—, (CH₂)—C(O)O—, —OC(O)—(CH₂)ₙ—, —(CH₂), —OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

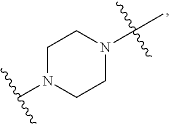 [SMILES: *N1CCN(*)CC1]

then
i) at least one of X¹, X², and X³ is not —CH₂—; and/or
ii) at least one of R¹, R², R³, R⁴, and R₅ is —R″MR′.

In some embodiments, the compound is of any of formulae (AIVa)-(AIVh):

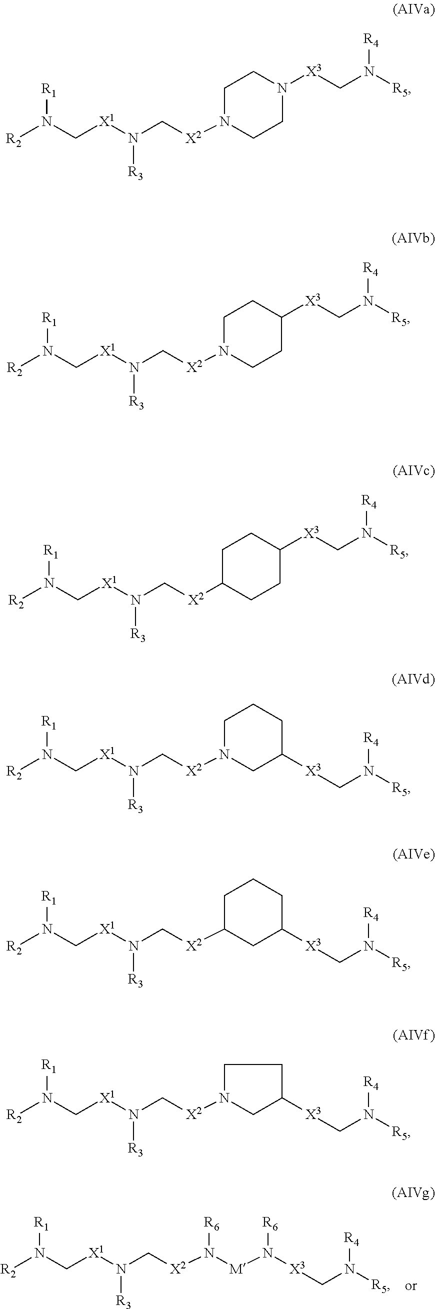

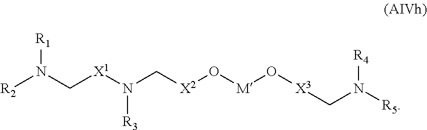

In some embodiments, the ionizable amino lipid is

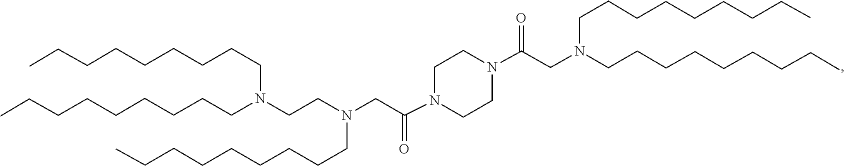 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH. Thus, a lipid may have a positive or partial positive charge at physiological pH.

### Formula (AV)

In some embodiments, the lipid nanoparticle comprises a lipid having the structure:

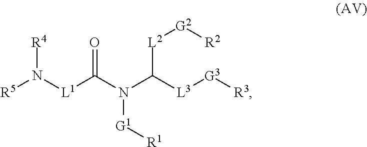

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)(C═O)O—, or —(C═O)—;
G² and G³ are each independently —(C═O)O— or —O(C═O)—; and n is an integer greater than 0.

### Formula (AVI)

In some embodiments, the lipid nanoparticle comprises a lipid having the structure:

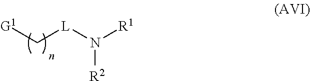

or a pharmaceutically acceptable salt, tautomer, or stereoisomer thereof, wherein:
G¹ is —N(R³)R⁴ or —OR₅;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R₅ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

### Formula (AVII)

In some embodiments, the lipid nanoparticle comprises a lipid having the structure:

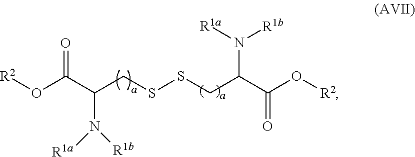

or a pharmaceutically acceptable salt thereof, wherein;
each R^{1a} is independently hydrogen, R^{1c}, or R^{1d};
each R^{1b} is independently R^{1c} or R^{1d},
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R² is —N(L₁-B)₂; —(OCH₂CH₂)ₙOH; or —(OCH₂CH₂)_{b}OCH₃;
each R³ and R⁴ is independently C₆-C₃₀ aliphatic;
each L₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

### Formula (AVIII)

In some embodiments, the lipid nanoparticle comprises a lipid having the structure:

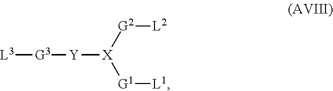

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(O)R¹, —OR¹, —S(O)ₓR¹, —S—SR¹, —C(═O)SR¹, —SC(═O)R¹, —NR^{a}C(═O)R¹, —C(═O)NR^{a}R^{b}, —NR^{a}C(═O)NR^{a}R^{e}, —OC(═O)NR^{a}R^{c}, or —NR^{d}C(═O)OR¹;
L² is —O(C—O)R², —(C═O)OR², —C(═O)R², —OR², —S(O)ₓR², —S—SR², —C(═O)SR², —SC(═O)R², —NR^{d}C(═O)R², —C(═O)NR^{a}R^{f}, —NR^{d}C(═O)NR^{a}R^{f}, —OC(═O)NR^{o}R^{f}; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

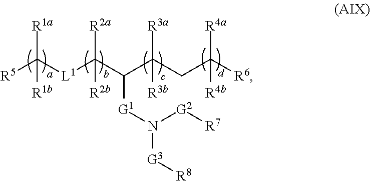

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ₋ₛ—S—S—, —C(═O)S—, —SC(═O)—, —NR^{a}C. (═O)—, —C(═O)NR^{a}—, —NR^{a}C(═O)NR^{a}—, —OC(═O)NR^{a}—, —NR^{a}C(═O)O— or a direct bond;
G¹ is C, —C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent RID and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C, —C₂₀ alkyl;
R⁸ is OH, —N(R⁹) (C═O) R¹⁰, —(C═O)NR⁹R¹⁰, —NR⁹R¹⁰, —(C═O)OR″¹ or —O(C═O)R″,
provided that G³ is C₄-C₆ alkylene when R₈ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

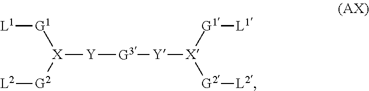

or a pharmaceutically acceptable salt, prodrug or stereoisomer thereof, wherein:
X and X′ are each independently N or CR;
Y and Y′ are each independently absent, —O(C═O)—, —(C═O)O)— or NR, provided that:
   a) Y is absent when X is N;
   b) Y′ is absent when X′ is N;
   c) Y is —O(C═O)—, —(C═O)O— or NR when X is CR; and
   d) Y′ is —O(C═O)—, —(C═O)O— or NR when X′ is CR,
L¹ and L² are each independently —O(C═O)R′, —(C═O)OR′, —C(═O)R′, —OR¹, —S(O)ₓR′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c}, or —NR^{a}C(═O)OR′;
L² and L² are each independentl y—O(C═O)R², —(C═O)OR², —C(═O)R², —OR², —S(O)₂R², —S—SR², —C(═O)SR², —SC(═O)R², —NR^{a}C(═O)R², —C(═O)NR^{a}R^{f}, —NR^{a}C(═O)NR^{b}R^{f}, —OC(═O)NR^{c}R^{f}; —NR^{a}C(═O)OR² or a direct bond to R²;
G¹, G¹″, G² and G²′ are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkonyl;
R^{c} and R^{f} are, at each occurrence, independently C₁-C₁₂ alkyl or C₂-C₁₂ alkenyl; R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

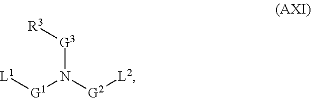

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{d}R^{c}, —NR^{a}C(═O)NR^{c}R^{e}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{a}C(═O)R², —C(═O)NR^{c}R^{f}, —NR^{e}C(═O)NR^{e}R^{f}, —OC(═O)NR^{d}R^{f}; —NR^{e}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{a} and Rₑ are each independently H or C₁-C₁₂ alkyl or C₁-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R³;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

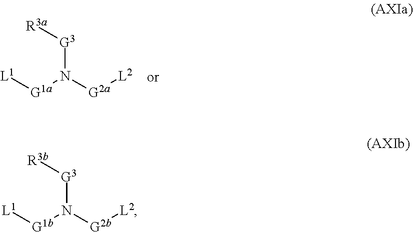

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, C(═O)SR¹, —SC(═O)R^{f}, —NR^{e}C(═O)R^{f}, —C(═O)NR^{b}R^{e}, —NR^{a}C(═O)NR^{b}R^{e}, —OC(═O)NR^{b}R^{f} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{e}C(═O)R^{f}, —C(═O)NR^{e}R^{f}, —NR^{c}C(═O)NR^{e}R^{f}, —OC(═O)NR^{e}R^{f}; —NR^{e}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a}) R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R₅ₐ is H, C₁-C₆ alkyl or C₂-C₅ alkenyl;
R_{5b} is C₂-C₁₂ alkyl or C₂-C₁₂ alkenyl when R⁴ is H; or R_{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

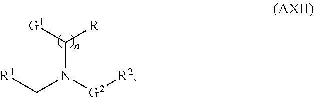

or a pharmaceutically acceptable salt, prodrug or stereoisomer thereof, wherein:
G¹ is —OH, —R³R⁴, —(C═O) R⁵ or —R³ (C═O) R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R₅ is optionally substituted straight or branched, saturated or unsaturated C₁-C₆ alkyl; and
n is an integer from 2 to 6.

### Formula (AXIII)

In some embodiments, the lipid nanoparticle comprises a lipid having the structure:

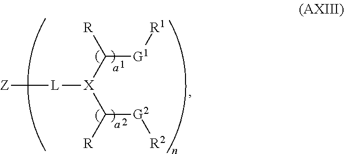

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a}) C(═O)—, —C(═O)N(R^{a})—, —N(R^{a}) C(═O)N(R^{a})—, —OC(═O)N(R^{a})— or —N(R^{a}) C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a}) C(═O)—, —C(═O)N(R^{a})—, —N(R^{a}) C(═O)N(R^{a})—, —OC(═O)N(R^{a})— or —N(R³) C(═O)O— or a direct bond;
L is, at each occurrence, ˜O(C═O)—, wherein ˜ represents a covalent bond to X; X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C ₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

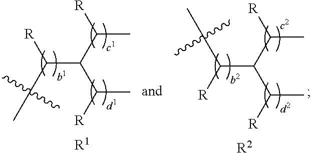

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

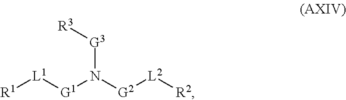

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a} C(═O)—, —C(═O)R^{a}, R^{a} C(═O)R^{a}—, —OC(═O)R^{a}— or —R^{a} C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—,, R^{a} C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR₅, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

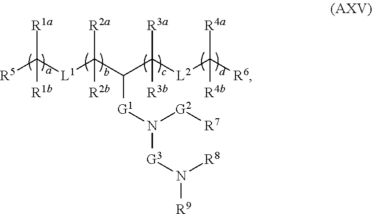

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a} C(═O)—, —C(═O)R^{a}—, —R⁵C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a} C(═O)— or a direct bond:
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b)
R^{1a} is H or C₁-C₁₂ alkyl, and R^{1b} together with the carbon atom to which it is bound is taken together with an adjacent R^{1d} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁶ and R⁹, together with the nitrogen atom to which they are attached, form a 5, 6 or 7-membered heterocyclic ring;
a, b, c and d are each independently an integer from 1 to 24; and x is 0, 1 or 2.

### Formula (AXVI)

In some embodiments, the lipid nanoparticle comprises a lipid having the structure:

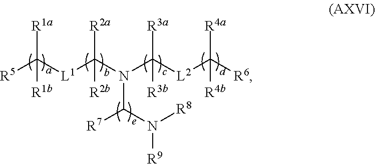

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R²⁵ together with the carbon atom to which it is bound is taken together with an adjacent R² and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R³⁶ and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

### Formula (AXVII)

In some embodiments, the lipid nanoparticle comprises a lipid having the structure:

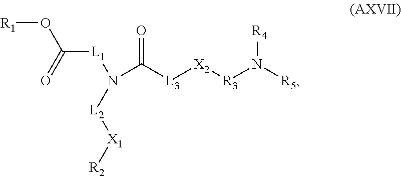

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R² is formed,
X₂ is S or O,
L₃ is a bond or a lower alkyl, or form a heterocycle with N,
R₃ is a lower alkyl, and

R₄ and R₅ are the same or different, each a lower alkyl.

### Compounds (AI)-(AII)

In some embodiments, the lipid nanoparticle comprises an ionizable lipid having the structure:

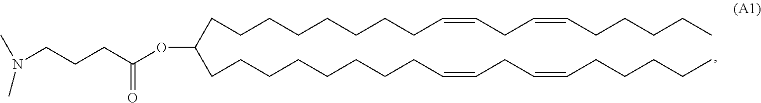 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

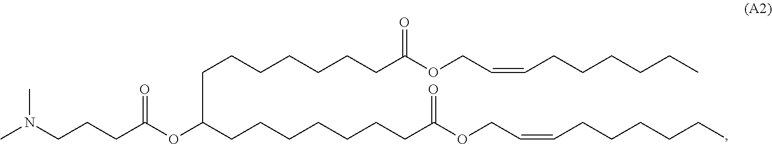

or a pharmaceutically acceptable salt thereof.

In some embodiments, the lipid nanoparticle comprises a lipid having the structure;

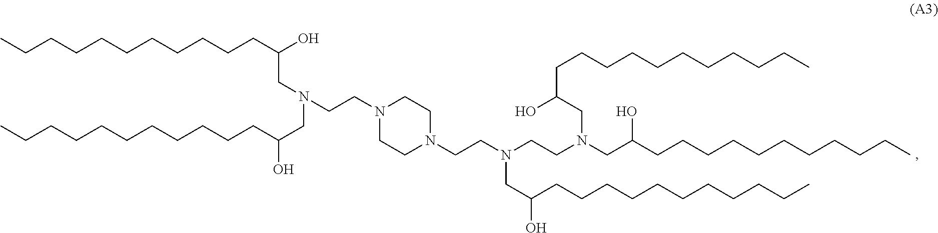 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

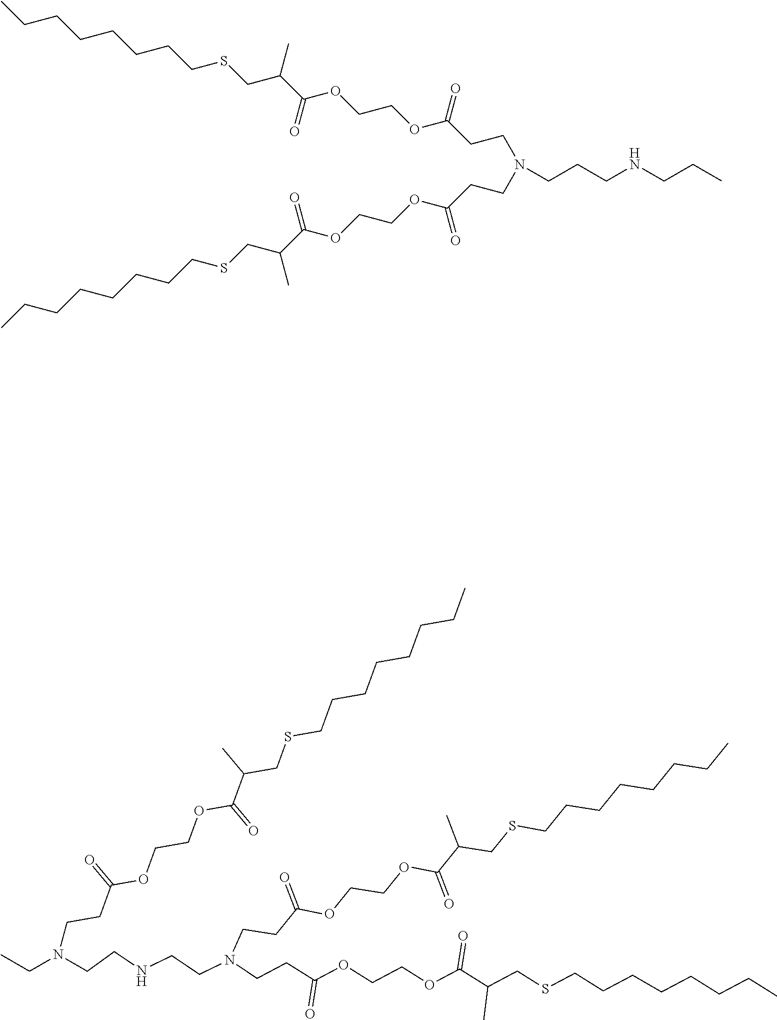

(A4), or a pharmaceutically acceptable salt thereof.

In some embodiments, the lipid nanoparticle comprises a lipid having the structure:

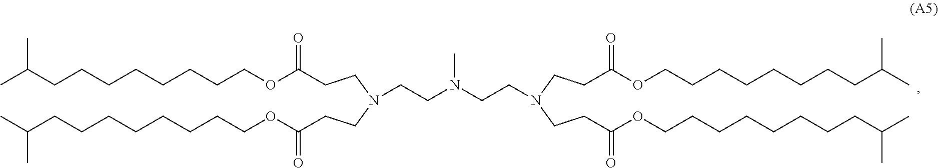 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

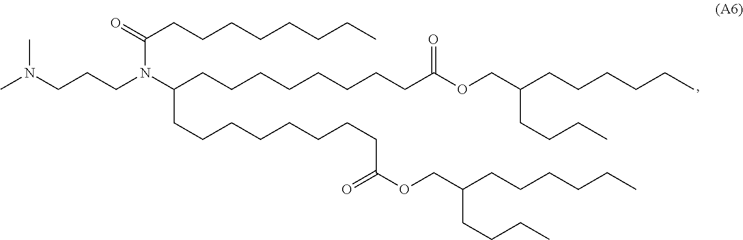 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

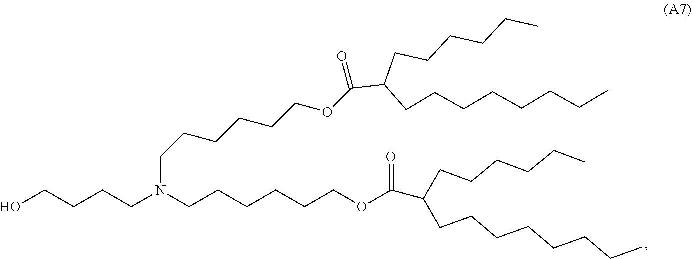 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

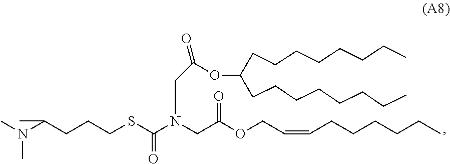 [SMILES: CCCCCC/C=C\COC(=O)CN(CC(=O)OC(CCCCCCCC)CCCCCCCC)C(=O)SCCCC(C)N(C)C]

or a pharmaceutically acceptable salt thereof.

In some embodiments, the lipid nanoparticle comprises a lipid having the ure:

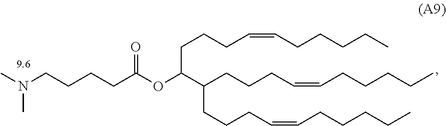 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

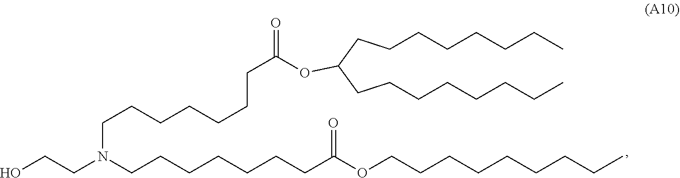 [SMILES: CCCCCCCCCOC(=O)CCCCCCCN(CCO)CCCCCCCC(=O)OC(CCCCCCCC)CCCCCCCC]

or a pharmaceutically acceptable salt thereof.
In some embodiments, the lipid nanoparticle comprises a lipid having the structure:

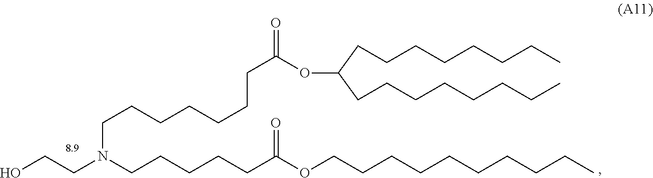 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-olcoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C₁₋₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C₁₋₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI);
or a salt thereof, wherein:

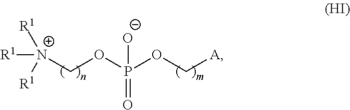

each R¹ s independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;

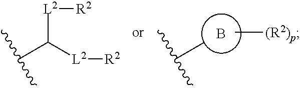

A is of the formula:
each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N}) N(R^{N}), NR^{N}C(═NR^{N}), NR^{N}C(═NR) N(R^{N}), C(S), C(S) N(R^{N}), NR^{N}C(S), NR^{N}C(S) N(R^{N}), S(O), OS(O), S(O)O, OS(O)O, OS(O)₂, S(O)₂O, OS(O)₂O, N(R^{N}) S(O), —S(O)N(R^{N}), N(R^{N})S(O)N(R^{N}), OS(O)N(R^{N}), N(R^{N}) S(O)O, S(O)₂, N(R^{N})S(O)₂, S(O)₂N(R^{N}), N(R^{N})S(O)₂N(R^{N}), OS(O)₂N(R^{N}), or N(R^{N}) S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

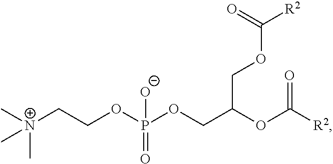

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

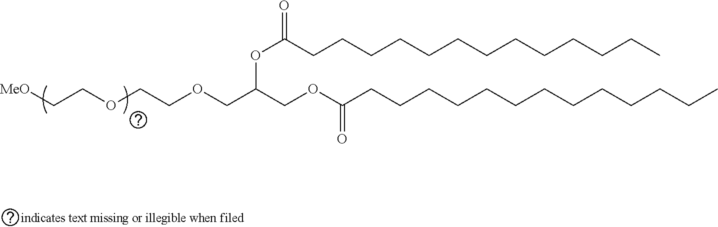

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG lipid is a compound of Formula (PI):

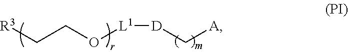

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, —OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;

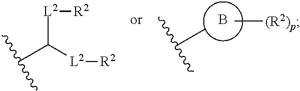

A is of the formula:
each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}), NR^{N}C(═NR) N(R^{N}), C(S), C(S) N(R^{N}), NR^{N}C(S), NR^{N}C(S) N(R^{N}), S(O), OS(O), S(O)O, OS(O)O, OS(O)₂, S(O)₂O, OS(O)₂O, N(R^{N}) S(O), —S(O)N(R^{N}), N(R^{N})S(O)N(R^{N}), OS(O)N(R^{N}), N(R^{N}) S(O)O, S(O)₂, N(R^{N}) S(O)₂, S(O)₂N(R^{N}), N(R^{N}) S(O)₂N(R^{N}), OS(O)₂N(R^{N}), or N(R^{N}) S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PD) is of Formula (PI-OH):

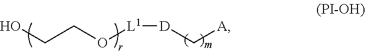

or a salt thereof.

### Formula (PII)

In certain embodiments, a PEG lipid is a PEGylated fatty acid. In certain embodiments, a PEG lipid is a compound of Formula (PII). In some embodiments, compounds of Formula (PII) have the following formula:

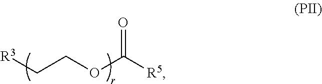

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R₅ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), —NR^{N}C(O)O, C(O)S, SC(O), C(═NR^{N}), C(═NR^{N}) N(R^{N}), NR^{N}C(═NR^{N}), NR^{N}C(═NR^{N}) N(R^{N}), C(S), C(S) N(R^{N}), NR^{N}C(S), NR^{N}C(S) N(R^{N}), S(O), OS(O), S(O)O, OS(O)O, OS(O)₂, —S(O)₂O, OS(O)₂O, N(R^{N}) S(O), S(O)N(R^{N}), N(R^{N}) S(O)N(R^{N}), OS(O)N(R^{N}), N(R^{N}) S(O)O, S(O)₂, N(R^{N}) S(O)₂, S(O)₂N(R^{N}), N(R^{N}) S(O)₂N(R^{N}), OS(O)₂N(R^{N}), or N(R^{N}) S(O)₂O; and
each instance of R^{N} is independently hydrogen, optionally substituted alkyl, or a nitrogen protecting group.

In certain embodiments, the compound of Formula (PII) is of Formula (PII-OH):

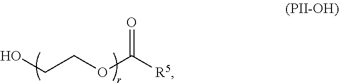

or a salt thereof. In some embodiments, r is 40-50.

In yet other embodiments the compound of Formula (PII) is:

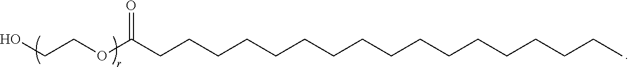

or a salt thereof.

In some embodiments, the compound of Formula (PII) is

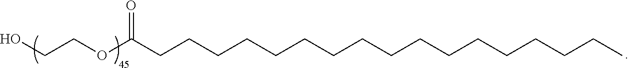

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). In embodiments comprise adding about 0.5 mo % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP comprises an ionizable amino lipid of Compound 1, wherein the non-cationic lipid is DSPC, the structural lipid that is cholesterol, and the PEG lipid is DMG-PEG.

In some embodiments, a LNP comprises an ionizable amino lipid of Compound 2, wherein the non-cationic lipid is DSPC, the structural lipid that is cholesterol, and the PEG lipid is DMG-PEG.

In some embodiments, a LNP comprises an ionizable amino lipid of any of Formula (AIII), (AIV), or (AV), a phospholipid comprising DSPC, a structural lipid, and a PEG lipid comprising PEG-DMG.

In some embodiments, a LNP comprises an ionizable amino lipid of any of Formula (AIII), (AIV), or (AV), a phospholipid comprising DSPC, a structural lipid, and a PEG lipid comprising a compound having Formula (PIJ).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG lipid comprising a compound having Formula (PII).

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

A lipid composition may comprise one or more lipids as described herein. Such lipids may include those useful in the preparation of lipid nanoparticle formulations as described above or as known in the art.

### Stabilizing Compounds

Some embodiments of the compositions described herein are stabilized pharmaceutical compositions. Various non-viral delivery systems, including nanoparticle formulations, present attractive opportunities to overcome many challenges associated with mRNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

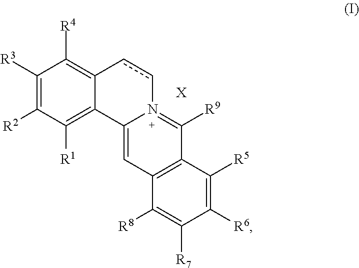

or a tautomer or solvate thereof, wherein:
[symbol] is a single bond or a double bond;
R¹ is H; R² is OCH₃, or together with R³ is OCH₂O; R³ is OCH₃, or together with R² is OCH₂O; R⁴ is H; R⁵ is H or OCH₃; R⁶ is OCH₃; R⁷ is H or OCH₃; R⁸ is H; R⁹ is H or CH₃; and X is a pharmaceutically acceptable anion, e.g., a halide such as chloride.

In some embodiments, the compound of Formula (I) has the structure of:

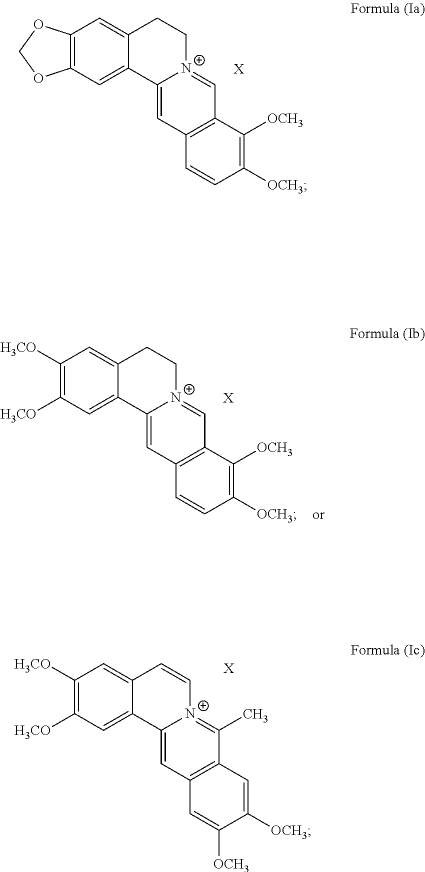

or a tautomer or solvate thereof.

In some embodiments, the stabilized pharmaceutical composition comprises a nucleic acid formulation comprising a nucleic acid and a lipid, and a compound of Formula (II):

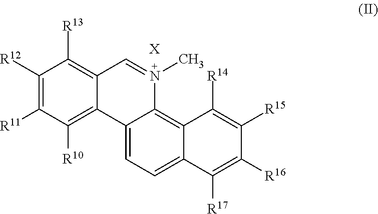

or a tautomer or solvate thereof, wherein:
R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

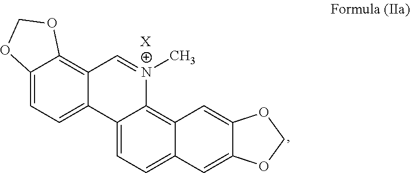

or a tautomer or solvate thereof.

Stabilizing compounds of Formulas (I), (Ia), (Ib), (Ic), (II), and (IIa) are described in International Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 103 M (e.g., less than 104 M, less than 105 M, less than 10⁻⁵ M, less than 10⁻⁷ M, less than 10⁻⁸ M, or less than 10⁻⁹ M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 M, 10 UM, 15 μM, 20 μM, 25 μM, 30 μM, 35 μM, 40 μM, 45 μM, 50 M, 60 μM, 70 μM, 80 μM, 90 μM, or 100 μM.

### EXAMPLES

### Example 1: Chemical Stability of CpA Dinucleotide in mRNA

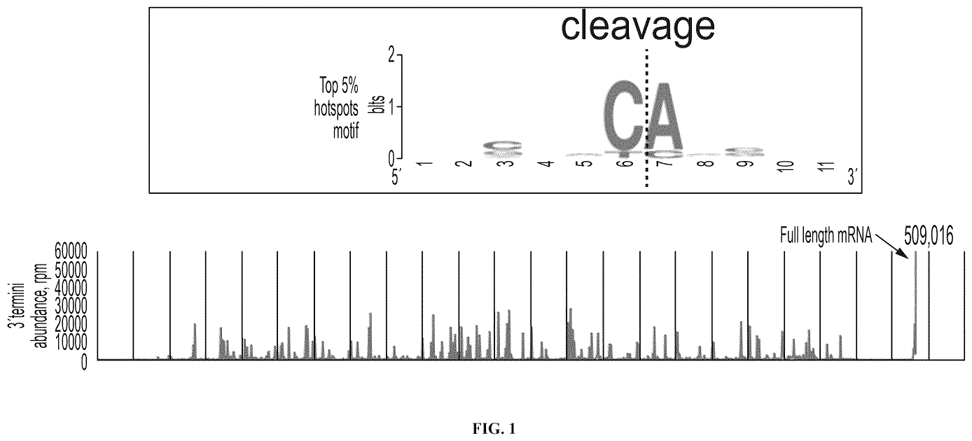

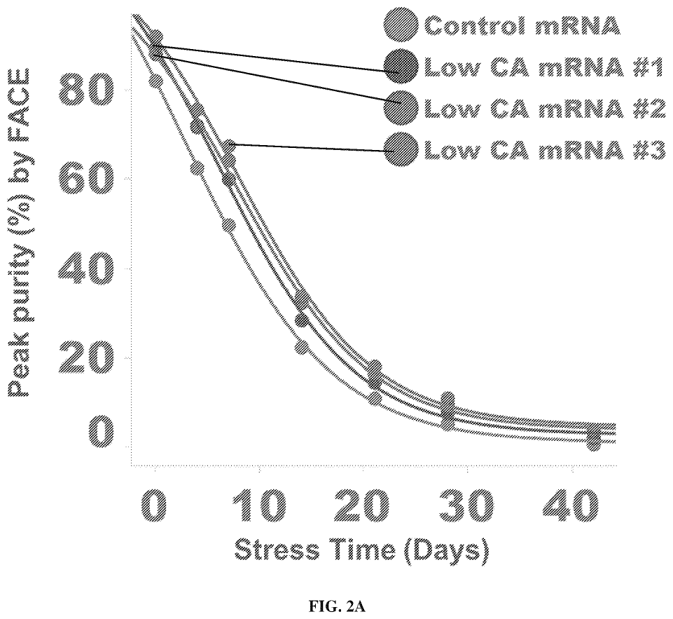

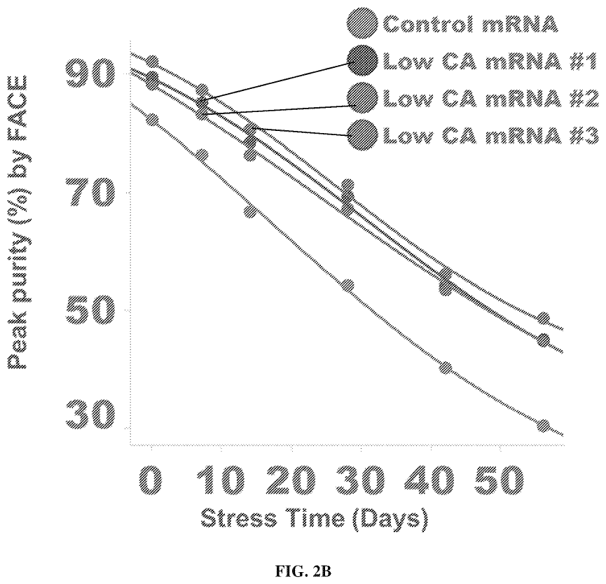

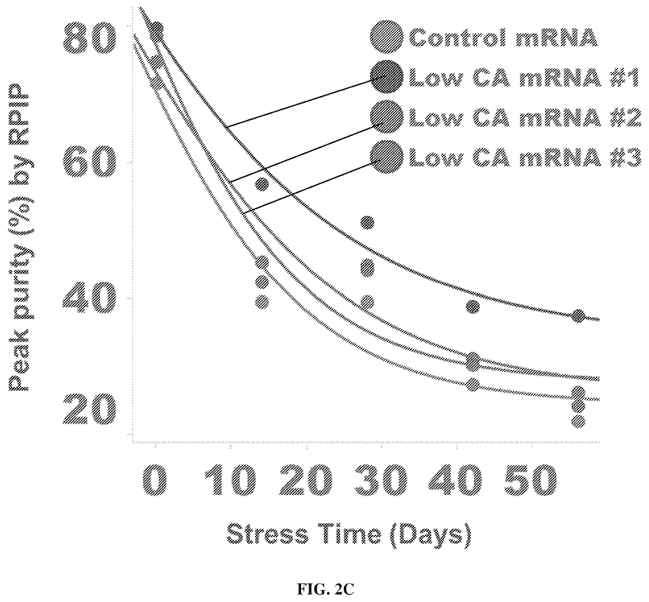

The susceptibility of different dinucleotide pairs to spontaneous cleavage was analyzed by incubating a test mRNA in water for 4 hours, and analyzing the resulting mRNA cleavage fragments by Illumina 3′ end sequencing. After incubation, fragments were sequenced, and reads were aligned to the reference sequence, with the 3′ nucleotide of each read corresponding to the first nucleotide in a dinucleotide pair that was cleaved to generate the sequenced mRNA fragment (e.g., a read ending in AAGCAC (SEQ 1D NO: 1) that aligned to the sequence AAGCACAAUC (SEQ 1D NO: 2) indicated that the bolded CpA dinucleotide was cleaved to generate the 3′ of the mRNA fragment). Analysis of the resulting abundance of cleaved dinucleotides indicated that the CpA dinucleotide was the most represented dinucleotide, indicating that this dinucleotide is particularly susceptible to cleavage (FIG. 1).

Next, a panel of mRNAs, each encoding the same antigen (Ag) with the same amino acid sequence, but varying in CpA dinucleotide content, was generated to test the effects of CpA dinucleotide content on stability during mRNA storage. Control mRNAs contained open reading frames with 366 CpA dinucleotides, while others (“Low CA”) contained open reading frames with only 79 CpA dinucleotides. Low CA mRNAs #2 and 3 contained increased % G/C content, relative to Low CA mRNA #1, and Low CA mRNAs #2 and #3 differed in 5′ UTR sequences. For each mRNA, the CpA dinucleotide content (# of CpA dinucleotides in the open reading frame), % G/C content (in mRNA sequence), and time to 50% purity during storage at (i)₄₀° C. unformulated; (ii)₂₅° C. unformulated; or (iii)₂₅° C. when formulated in a lipid nanoparticle (LNP), is shown in Table 1. At both temperatures, mRNAs having fewer CpA dinucleotides decayed more slowly than the control mRNA, indicating that the stability of a given mRNA may be increased by reducing the abundance of CpA dinucleotides.

TABLE 1
Stability of mRNAs with low CpA dinucleotide content

| | | | Time to 50% mRNA purity (days) | | |
|---|---|---|---|---|---|
| | # CpAs | | 40° C. | 25° C. | 25° |
| | in | | (mRNA | (mRNA | (LNP- |
| mRNA | ORF | % G/C | alone) | alone) | mRNA) |
| Control | 366 | 62 | 6.0 | 30.5 | 12.3 |
| mRNA | | | | | |
| Low CA | 79 | 52 | 9.0 | 49.4 | 29.0 |
| mRNA #1 | | | | | |
| Low CA | 79 | 60 | 8.3 | 48.6 | 17.6 |
| mRNA #2 | | | | | |
| Low CA | 79 | 60 | 9.2 | 54.2 | 17.0 |
| mRNA #3 | | | | | |

### Example 2: In Vitro Expression and In Vivo Immunogenicity of mRNAs with Low CpA Dinucleotide Content

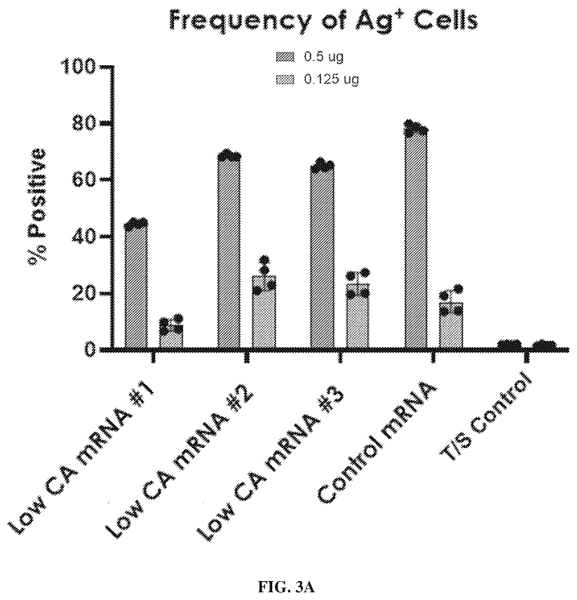

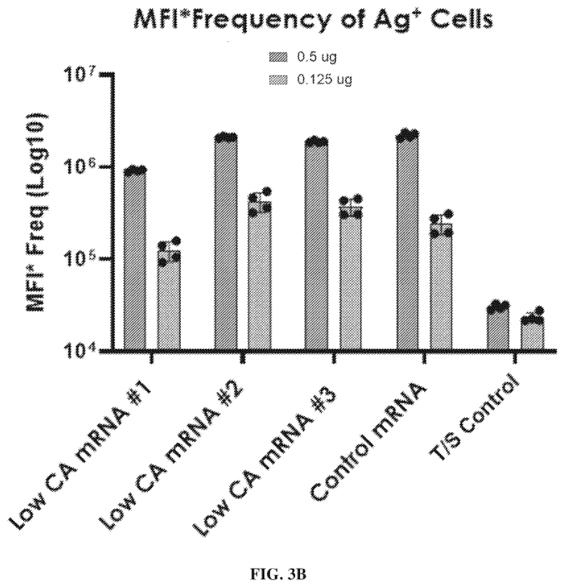

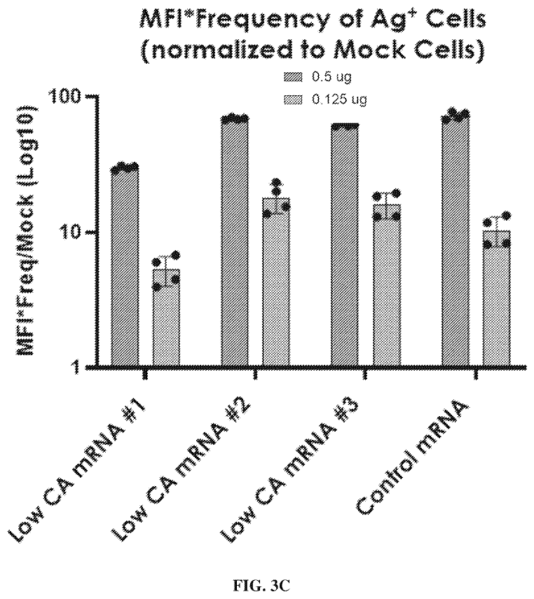

The panel of mRNAs tested in Example 1 was also tested in cultured EXPI293 cells to evaluate expression of mRNAs with reduced CpA dinucleotide content. Following addition of LNP-mRNA compositions to cells and sufficient time to allow antigen expression, cells were collected, stained with an Ag-specific antibody, and analyzed by flow cytometry to evaluate antigen expression. The results of this analysis are shown in FIGS. 3A-3C. All Low CA mRNA compositions allowed translation of the encoded antigen in cells, with at least 40% of cells expressing detectable antigen (FIG. 3A), and total protein expression being similar to that of cells contacted with compositions containing control mRNAs (FIGS. 3B and 3C).

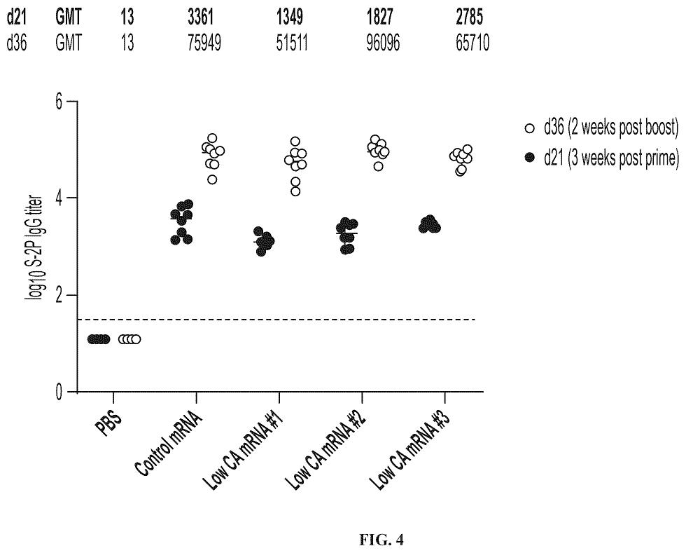

The same panel of mRNA vaccine compositions were tested in C57BL/6 mice. Mice were immunized with two doses of a composition containing 1 μg mRNA, receiving the first dose on day 0 and the second dose on day 22. On day 21, three weeks after the first dose, and day 36, two weeks after the second dose, sera were collected to evaluate antibody responses elicited by each LNP-mRNA composition. The results of ELISAs, measuring titers of antibodies specific to the encoded antigen, are shown in FIG. 4. These results indicate that reduction of CpA dinucleotide content may be used to improve mRNA stability, while still allowing expression in vitro and in vivo (e.g., sufficient expression to elicit an antibody response to an encoded antigen).

### Example 3: In Vitro Transcription (IVT)

### Materials and Methods

Alternative mRNAs are made using standard laboratory methods and materials for in vitro transcription. The open reading frame (ORF) of the gene of interest may be flanked by a 5′ untranslated region (UTR) containing a strong Kozak translational initiation signal, and an alpha-globin 3′ UTR.

The ORF may also include various upstream or downstream additions (such as, but not limited to, β-globin, tags, etc.) may be ordered from an optimization service such as, but limited to, DNA2.0 (Menlo Park, Calif.) and may contain multiple cloning sites which may have XbaI recognition. Upon receipt of the construct, it may be reconstituted and transformed into chemically competent E. coli. NEB DH5-alpha Competent E. coli may be used. Transformations are performed according to NEB instructions using 100 ng of plasmid. The protocol is as follows:
Thaw a tube of NEB 5-alpha Competent E. coli cells on ice for 10 minutes.
Add 1-5 μl containing 1 μg-100 ng of plasmid DNA to the cell mixture. Carefully flick the tube 4-5 times to mix cells and DNA. Do not vortex.
Place the mixture on ice for 30 minutes. Do not mix.
Heat shock at 42° C. for exactly 30 seconds. Do not mix.
Place on ice for 5 minutes. Do not mix.
Pipette 950 μl of room temperature SOC into the mixture.
Place at 37° C. for 60 minutes. Shake vigorously (250 rpm) or rotate.
Warm selection plates to 37° C.
Mix the cells thoroughly by flicking the tube and inverting.
Spread 50-100 μl of each dilution onto a selection plate and incubate overnight at 37° C. Alternatively, incubate at 30° C. for 24-36 hours or 25° C. for 48 hours.

A single colony is then used to inoculate 5 m1 of LB growth media using the appropriate antibiotic and then allowed to grow (250 RPM, 37° C.) for 5 hours. This is then used to inoculate a 200 m1 culture medium and allowed to grow overnight under the same conditions.

To isolate the plasmid (up to 850 μg), a maxi prep is performed using the Invitrogen PURELINK™ HiPure Maxiprep Kit (Carlsbad, Calif.), following the manufacturer's instructions.

In order to generate cDNA for In Vitro Transcription (IVT), the plasmid is first linearized using a restriction enzyme such as XbaI. A typical restriction digest with XbaI will comprise the following: Plasmid 1.0 μg; 10× Buffer 1.0 μl; XbaI 1.5 μl; dH2O up to 10 μl; incubated at 37° C. for 1 hr. If performing at lab scale (<5 μg), the reaction is cleaned up using Invitrogen's PURELINK™ PCR Micro Kit (Carlsbad, Calif.) per manufacturer's instructions. Larger scale purifications may need to be done with a product that has a larger load capacity such as Invitrogen's standard PURELINK™ PCR Kit (Carlsbad, Calif.). Following the cleanup, the linearized vector is quantified using the NanoDrop and analyzed to confirm linearization using agarose gel electrophoresis.

### IVT Reaction

The in vitro transcription reaction generates mRNA containing alternative nucleotides or alternative RNA. The input nucleotide triphosphate (NTP) mix is made in-house using natural and unnatural NTPs.

A typical in vitro transcription reaction includes the following:

| Template cDNA | 1.0 | μg |
|---|---|---|
| 10x transcription buffer (400 mM Tris-HCl | 2.0 | μl |
| pH 8.0, 190 mM MgCl2, 50 mM DTT, 10 mM Spermidine) | | |
| Custom NTPs (25 mM each) | 7.2 | μl |
| RNase Inhibitor | 20 | U |
| T7 RNA polymerase | 3000 | U |
| dH2O | up to 20.0 μl | |
| Incubation at 37° C. for 3 hr-5 hrs. | | |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

The T7 RNA polymerase may be selected from, T7 RNA polymerase, T3 RNA polymerase and mutant polymerases such as, but not limited to, the novel polymerases able to incorporate alternative NTPs as well as those polymerases described by Liu (Esvelt et al. (Nature (2011)₄₇₂ (7344): 499-503 and U.S. Publication No. US 2011/0177495) which recognize alternate promoters, Ellington (Chelliserrykattil and Ellington, Nature Biotechnology (2004)₂₂ (9): 1155-1160) describing a T7 RNA polymerase variant to transcribe 2′-O-methyl RNA and Sousa (Padilla and Sousa, Nucleic Acids Research (2002) 30(24): e128) describing a T7 RNA polymerase double mutant; herein incorporated by reference in their entireties.

Agarose Gel Electrophoresis of Alternative mRNA

Individual alternative mRNAs (200-400 ng in a 20 μl volume) are loaded into a well on a non-denaturing 1.2% Agarose E-Gel (Invitrogen, Carlsbad, Calif.) and run for 12-15 minutes according to the manufacturer protocol.

### Agarose Gel Electrophoresis of RT-PCR Products

Individual reverse transcribed-PCR products (200-400 ng) are loaded into a well of a non-denaturing 1.2% Agarose E-Gel (Invitrogen, Carlsbad, Calif.) and run for 12-15 minutes according to the manufacturer protocol.

Nanodrop Alternative mRNA Quantification and UV Spectral Data

Alternative mRNAs in TE buffer (1 μl) are used for Nanodrop UV absorbance readings to quantitate the yield of each alternative mRNA from an in vitro transcription reaction (UV absorbance traces are not shown).

### Example 3: Enzymatic Capping of mRNA

Capping of the mRNA is performed as follows where the mixture includes: IVT RNA 60 μg-180 μg and dH2O up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400 U); Vaccinia capping enzyme (Guanylyl transferase) (40 U); dH2O(Up to 28 μl); and incubation at 37° C. for 30 minutes for 60 μg RNA or up to 2 hours for 180 μg of RNA.

The mRNA is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 4: 5′-Guanosine Capping

### Materials and Methods

The cloning, gene synthesis and vector sequencing may be performed by DNA2.0 Inc. (Menlo Park, Calif.). The ORF is restriction digested using XbaI and used for cDNA synthesis using tailed- or tail-less-PCR. The tailed-PCR cDNA product is used as the template for the alternative mRNA synthesis reaction using 25 mM each alternative nucleotide mix (all alternative nucleotides may be custom synthesized or purchased from TriLink Biotech, San Diego, Calif. except pyrrolo-C triphosphate which may be purchased from Glen Research, Sterling Va.; unmodified nucleotides are purchased from Epicenter Biotechnologies, Madison, Wis.) and CellScript MEGASCRIPT™ (Epicenter Biotechnologies, Madison, Wis.) complete mRNA synthesis kit.

The in vitro transcription reaction is run for 4 hours at 37° C. Alternative mRNAs incorporating adenosine analogs are poly (A) tailed using yeast Poly (A) Polymerase (Affymetrix, Santa Clara, Calif.). The PCR reaction uses HiFi PCR 2× MASTER MIX™ (Kapa Biosystems, Woburn, Mass.). Alternative mRNAs are post-transcriptionally capped using recombinant Vaccinia Virus Capping Enzyme (New England BioLabs, Ipswich, Mass.) and a recombinant 2′-O-methyltransferase (Epicenter Biotechnologies, Madison, Wis.) to generate the 5′-guanosine Cap1 structure. Cap 2 structure and Cap 2 structures may be generated using additional 2′-O-methyltransferases. The in vitro transcribed mRNA product is run on an agarose gel and visualized. Alternative mRNA may be purified with Ambion/Applied Biosystems (Austin, Tex.) MEGAClear RNA™ purification kit. The PCR uses PURELINK™ PCR purification kit (Invitrogen, Carlsbad, Calif.). The product is quantified on NANODROP™ UV Absorbance (ThermoFisher, Waltham, Mass.). Quality, UV absorbance quality and visualization of the product was performed on an 1.2% agarose gel. The product is resuspended in TE buffer.

5′-Capping Alternative Nucleic Acid (mRNA) Structure

5′-capping of alternative mRNA may be completed concomitantly during the in vitro-transcription reaction using the following chemical RNA cap analogs to generate the 5′-guanosine cap structure according to manufacturer protocols: 3″-O-Me-m7G(5′)ppp(5′)G (the ARCA cap); G(5′)ppp(5′)A; G(5′)ppp(5′)G; m7G(5′)ppp(5′)A; m7G(5′)ppp(59)G (New England BioLabs, Ipswich, Mass.). 5′-capping of alternative mRNA may be completed post-transcriptionally using a Vaccinia Virus Capping Enzyme to generate the “Cap 0” structure: m7G(5′)ppp(5′) G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-0 methyl-transferase. Enzymes are preferably derived from a recombinant source.

When transfected into mammalian cells, the alternative mRNAs have a stability of 12-18 hours or more than 18 hours, e.g., 24, 36, 48, 60, 72 or greater than 72 hours,

### Example 5: In Vivo Expression of Selected Sequences

Lipid nanoparticles containing modified or unmodified mRNA are administered to mice at mRNA doses of at 0.05 mg/kg intravenously, subcutaneous, or intramuscularly. Expression of polypeptides encoded mRNAs is evaluated by any method known in the art. For example, expression of encoded fluorescent protein may be evaluated by isolating cells and measuring fluorescence intensity by fluorescence activated cell sorting (FACS) or fluorescent microscopy.

### Example 6: Method of Screening for Protein Expression

### Electrospray Ionization

A biological sample which may contain proteins encoded by modified RNA administered to the subject is prepared and analyzed according to the manufacturer protocol for electrospray ionization (ESI) using 1, 2, 3 or 4 mass analyzers. A biologic sample may also be analyzed using a tandem ESI mass spectrometry system.

Patterns of protein fragments, or whole proteins, are compared to known controls for a given protein and identity is determined by comparison.

### Matrix-Assisted Laser Desorption/Ionization

A biological sample which may contain proteins encoded by alternative RNA administered to the subject is prepared and analyzed according to the manufacturer protocol for matrix-assisted laser desorption/ionization (MALDI).

Patterns of protein fragments, or whole proteins, are compared to known controls for a given protein and identity is determined by comparison.

### Liquid Chromatography-Mass Spectrometry-Mass Spectrometry

A biological sample, which may contain proteins encoded by alternative RNA, may be treated with a trypsin enzyme to digest the proteins contained within. The resulting peptides are analyzed by liquid chromatography-mass spectrometry-mass spectrometry (LC/MS/MS). The peptides are fragmented in the mass spectrometer to yield diagnostic patterns that can be matched to protein sequence databases via computer algorithms. The digested sample may be diluted to achieve 1 ng or less starting material for a given protein, Biological samples containing a simple buffer background (e.g., water or volatile salts) are amenable to direct in-solution digest; more complex backgrounds (e.g., detergent, non-volatile salts, glycerol) require an additional clean-up step to facilitate the sample analysis.

Patterns of protein fragments, or whole proteins, are compared to known controls for a given protein and identity is determined by comparison.

### Example 7: In Vivo Assays with Human EPO Containing Alternative Nucleotides Formulation

Modified mRNAs encoding human erythropoietin (hEPO) are formulated in lipid nanoparticles (LNPs) comprising DLin-KC2-DMA, DSPC, Cholesterol, and PEG-DMG at 50:10:38.5:1.5 mol % respectively. The LNPs are made by direct injection utilizing nanoprecipitation of ethanol solubilized lipids into a pH 4.0 50 mM citrate mRNA solution. The EPO LNP particle size distributions are characterized by DLS. Encapsulation efficiency (EE) is determined using a Ribogreen™ fluorescence-based assay for detection and quantification of nucleic acids.

| Lipid Class | Lipid | Lipid/mol % |
|---|---|---|
| Ionizable Lipid | 2-(2,2-di((9Z,12Z)- | 50 |
| | octadeca-9,12-dien-1yl)-1,3- | |
| | diocolan-4-yl)-N,N- | |
| | dimethylethanamine | |
| | (DLin-KC2-DMA) | |
| Phospholipid | 1,2-distearoyl-sn-glycero-3- | 10 |
| | phosphocholine | |
| | (DSPC) | |
| Cholesterol | cholest-5-en-3β-ol | 38.5 |
| | (Cholesterol) | |
| PEG Lipid | 1,2-Dimyristoyl-sn- | 1.5 |
| | glycerol, | |
| | methoxypolyethylene glycol | |
| | (PEG-DMG) | |

### Methods

Female Balb/c mice (n=5) are administered 0.05 mg/kg IM (50 μl in the quadriceps) or IV (100 μl in the tail vein) of human EPO mRNA. At time 8 hours after the injection mice are euthanized and blood was collected in serum separator tubes. The samples are spun, and serum samples are then run on an EPO ELISA following the kit protocol (Stem Cell Technologies Catalog #01630).

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A non-naturally occurring mRNA encoding a polypeptide, the mRNA comprising an open reading frame (ORF) encoding the polypeptide,
wherein the ORF comprises a number of CpA dinucleotides that is greater than or equal to a theoretical minimum and less than or equal to 300% of the theoretical minimum.

2. A non-naturally occurring mRNA encoding a polypeptide, the mRNA comprising an open reading frame (ORF) encoding the polypeptide,
wherein the ORF comprises a number of CpA dinucleotides that is:
(i) greater than or equal to a theoretical minimum; and
(ii) no more than 11 CpA dinucleotides per 100 nucleotides of the ORF greater than the theoretical minimum.

3. The mRNA of claim 2, wherein the number of CpA dinucleotides per 100 nucleotides of the ORF greater than the theoretical minimum is no more than 10, no more than 9, no more than 8, no more than 7, no more than 6, no more than 5, no more than 4, no more than 3, no more than 2, or no more than 1.

4. A non-naturally occurring mRNA encoding a polypeptide, the mRNA comprising an open reading frame (ORF) encoding the polypeptide,
wherein the ORF comprises a CpA dinucleotide content of 6.5% or less.

5. The mRNA of claim 4, wherein the ORF comprises a CpA dinucleotide content of 6.0% or less, 5.5% or less, 5% or less, 4.5% or less, 4% or less, 3.5% or less, 3.0% or less, 2.5% or less, 2.0% or less, 1.5% or less, 1.0% or less, or 0.5% or less.

6. The mRNA of any one of the preceding claims, wherein:
(a) fewer than 30% of amino acids that immediately precede an isoleucine residue in the polypeptide are encoded by codons in the ORF that end in cytidine nucleotides;
(b) fewer than 30% of amino acids that immediately precede a methionine residue in the polypeptide are encoded by codons in the ORF that end in cytidine nucleotides;
(c) fewer than 30% of amino acids that immediately precede a threonine residue in the polypeptide are encoded by codons in the ORF that end in cytidine nucleotides;
(d) fewer than 30% of amino acids that immediately precede an asparagine residue in the polypeptide are encoded by codons in the ORF that end in cytidine nucleotides;
(e) fewer than 30% of amino acids that immediately precede a lysine residue in the polypeptide are encoded by codons in the ORF that end in cytidine nucleotides;
(f) fewer than 30% of amino acids that immediately precede a serine residue, wherein the serine residue is encoded by a codon in the ORF beginning with an adenosine nucleotide, are encoded by codons in the ORF that end in cytidine nucleotides; and/or
(g) fewer than 30% of amino acids that immediately precede an arginine residue, wherein the arginine residue is encoded by a codon in the ORF beginning with an adenosine nucleotide, are encoded by codons in the ORF that end in cytidine nucleotides.

7. The mRNA of any one of the preceding claims, wherein the nucleotide sequence of the mRNA comprises a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%.

8. The mRNA of any one of the preceding claims, wherein one or more nucleotides of the mRNA comprises a chemically modified nucleotide.

9. The mRNA of any one of the preceding claims, wherein each uridine nucleotide of the mRNA comprises a chemically modified nucleotide.

10. An mRNA encoding a polypeptide, the mRNA comprising an open reading frame (ORF) encoding the polypeptide,
wherein the mRNA has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%,
wherein each of the uridine nucleotides of the ORF comprises a chemical modification,
wherein:
(a) fewer than 30% of amino acids that immediately precede an isoleucine residue in the polypeptide are encoded by codons in the ORF that end in cytidine nucleotides;
(b) fewer than 30% of amino acids that immediately precede a methionine residue in the polypeptide are encoded by codons in the ORF that end in cytidine nucleotides;
(c) fewer than 30% of amino acids that immediately precede a threonine residue in the polypeptide are encoded by codons in the ORF that end in cytidine nucleotides;
(d) fewer than 30% of amino acids that immediately precede an asparagine residue in the polypeptide are encoded by codons in the ORF that end in cytidine nucleotides;
(e) fewer than 30% of amino acids that immediately precede a lysine residue in the polypeptide are encoded by codons in the ORF that end in cytidine nucleotides;
(f) fewer than 30% of amino acids that immediately precede a serine residue, wherein the serine residue is encoded by a codon in the ORF beginning with an adenosine nucleotide, are encoded by codons in the ORF that end in cytidine nucleotides; and/or
(g) fewer than 30% of amino acids that immediately precede an arginine residue, wherein the arginine residue is encoded by a codon in the ORF beginning with an adenosine nucleotide, are encoded by codons in the ORF that end in cytidine nucleotides.

11. The mRNA of any one of claim 8-9 or 10, wherein the chemically modified nucleotide comprise N1-methylpseudouridine.

12. The mRNA of any one of the preceding claims, wherein fewer than 15% of serine residues, fewer than 27% of proline residues, fewer than 28% of threonine residues, and fewer than 23% of alanine residues in the polypeptide are encoded by codons in the ORF comprising a CpA dinucleotide.

13. The mRNA of any one of the preceding claims, wherein:
(a) no serine residue of the polypeptide is encoded by a codon in the ORF comprising a CpA dinucleotide;
(b) no proline residue of the polypeptide is encoded by a codon in the ORF comprising a CpA dinucleotide;
(c) no threonine residue of the polypeptide is encoded by a codon in the ORF comprising a CpA dinucleotide; and/or
(d) no alanine residue of the polypeptide is encoded by a codon in the ORF comprising a CpA dinucleotide.

14. The mRNA of any one of the preceding claims, wherein:
(a) no amino acid that immediately precedes an isoleucine residue in the polypeptide is encoded by a codon in the ORF that ends in a cytidine nucleotide;
(b) no amino acid that immediately precedes a methionine residue in the polypeptide is encoded by a codon in the ORF that ends in a cytidine nucleotide;
(c) no amino acid that immediately precedes a threonine residue in the polypeptide is encoded by a codon in the ORF that ends in a cytidine nucleotide;
(d) no amino acid that immediately precedes an asparagine residue in the polypeptide is encoded by a codon in the ORF that ends in a cytidine nucleotide;
(e) no amino acid that immediately precedes a lysine residue in the polypeptide is encoded by a codon in the ORF that ends in a cytidine nucleotide;
(f) no amino acid that immediately precedes a serine residue, wherein the serine residue is encoded by a codon in the ORF beginning with an adenosine nucleotide, is encoded by a codon in the ORF that ends in a cytidine nucleotide; and/or
(g) no amino acid that immediately precedes an arginine residue, wherein the arginine residue is encoded by a codon in the ORF beginning with an adenosine nucleotide, is encoded by a codon in the ORF that ends in a cytidine nucleotide.

15. The mRNA of any one of the preceding claims, wherein no amino acid that immediately precedes an isoleucine, methionine, threonine, asparagine, or lysine residue in the polypeptide is encoded by a codon that ends in a cytidine nucleotide.

16. The mRNA of any one of the preceding claims, wherein no codon in the ORF beginning with an adenosine nucleotide is immediately preceded by a codon in the ORF that ends in a cytidine nucleotide.

17. The mRNA of any one of the preceding claims, wherein the ORF is codon-optimized for expression in a cell.

18. The mRNA of claim 17, wherein the cell is a mammalian cell.

19. The mRNA of any one of the preceding claims, wherein the mRNA further comprises:
(i) a 5′ untranslated region (UTR); and/or
(ii) a 3′ UTR.

20. The mRNA of claim 19, wherein the 5′ UTR is a heterologous UTR and/or the 3′ UTR is a heterologous UTR.

21. The mRNA of claim 19 or 20, wherein the 5′ UTR comprises five or fewer, four or fewer, three or fewer, two or fewer, one or fewer, or zero CpA dinucleotides.

22. The mRNA of any one of claims 19-21, wherein the 5′ UTR does not comprise a CpA dinucleotide.

23. The mRNA of any one of claims 19-22, wherein the 3′ UTR comprises five or fewer, four or fewer, three or fewer, two or fewer, one or fewer, or zero CpA dinucleotides.

24. The mRNA of any one of claims 19-23, wherein the 3′ UTR does not comprise a CpA dinucleotide.

25. The mRNA of any one of claims 19-24, wherein the last nucleotide of the 5′ UTR is not a cytidine nucleotide.

26. The mRNA of any one of claims 19-25, wherein the 5′ UTR has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%.

27. The mRNA of any one of claims 19-26, wherein the ORF has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%.

28. The mRNA of any one of claims 19-27, wherein the 3′ UTR has a % G/C content of 30-80%, 40%-70%, 50%-60%, 35%-50%, 50%-65%, 65%-70%, 40%-45%, 45%-50%, 50%-55%, 55%-70%, 70%-75%, or 75%-80%.

29. The mRNA of any of the preceding claims, wherein the mRNA further comprises:
(iii) a 5′ cap structure; and/or
(iv) a poly-A tail.

30. The mRNA of claim 29, wherein the last nucleotide of the 3′ UTR is not a cytidine nucleotide.

31. The mRNA of claim 29 or 30, wherein the 5′ cap structure comprises 7 mG(5′)ppp(5′)NlmpNp.

32. The mRNA of any one of the preceding claims, wherein the level of expression in a mammalian cell of the encoded polypeptide from the mRNA is at least 50% of the level of expression of a reference mRNA comprising a reference open reading frame (rORF) encoding the polypeptide, wherein the rORF comprises a higher number of CpA dinucleotides than the ORF.

33. The mRNA of any one of the preceding claims, wherein one or more CpA dinucleotides of the mRNA comprises a modified cytidine nucleotide and/or a modified adenosine nucleotide.

34. The mRNA of any one of the preceding claims, wherein the number of CpA dinucleotides comprising an unmodified cytidine nucleotide and an unmodified adenosine nucleotide in the ORF is 100%, 95% or less, 90% or less, 80% or less, 70% or less, 60% or less, 50% or less, 40% or less, 30% or less, 20% or less, or 10% or less of the total number of histidine and glutamine residues in the polypeptide.

35. The mRNA of any one of the preceding claims, wherein the polypeptide comprises 9-5,000, 20-4,000, 30-3,000, 40-2,000, or 50-1,500 amino acids.

36. The mRNA of any one of the preceding claims, wherein the polypeptide is a vaccine antigen or a therapeutic protein.

37. The mRNA of any one of the preceding claims, wherein a coefficient of degradation at 25° C. of the mRNA is 90% or less, 80% or less, 70% or less, 60% or less, or 50% or less, relative to an mRNA comprising a wild-type ORF encoding the polypeptide.

38. The mRNA of any one of the preceding claims, wherein a composition comprising a plurality of the mRNAs remains above 50% purity for at least 30 days, at least 60 days, at least 90 days, at least 120 days, at least 150 days, or at least 180 days longer in storage than a composition comprising a plurality of mRNAs comprising a wild-type ORF encoding the polypeptide.

39. The mRNA of claim 38, wherein storage of the mRNA is conducted at a temperature between about 2° C. to about 8° C.

40. The mRNA of claim 38 or 39, wherein the mRNA is stored in a buffer comprising 10-50 mM Tris and 5-10% sucrose, wherein the buffer has a pH of about 7.3 to about 7.6.

41. The mRNA of any one of the preceding claims, wherein the stability of the mRNA is increased relative to a reference mRNA having a higher number of CpA dinucleotides, the reference mRNA comprising a reference open reading frame (rORF) encoding the polypeptide, wherein the rORF has a higher number of CpA dinucleotides than the ORF.

42. A lipid nanoparticle comprising the mRNA of any one of the preceding claims, and an ionizable cationic lipid, a non-cationic lipid, a sterol, and a polyethylene glycol (PEG)-modified lipid.

43. The lipid nanoparticle of claim 42, wherein the lipid nanoparticle comprises 20-60% ionizable cationic lipid, and 5-25% non-cationic lipid, 25-55% cholesterol, and 0.5-15% polyethylene glycol (PEG)-modified lipid.

44. The lipid nanoparticle of claim 42 or 43, wherein a coefficient of degradation at 25° C. of the mRNA in the lipid nanoparticle is 90% or less, 80% or less, 70% or less, 60% or less, or 50% or less, relative to an mRNA comprising a wild-type ORF encoding the polypeptide.

45. The lipid nanoparticle of any one of claims 42-44, wherein a composition comprising a plurality of the lipid nanoparticles remains above 50% purity for at least 30 days, at least 60 days, at least 90 days, at least 120 days, at least 150 days, or at least 180 days longer in storage than a composition comprising a plurality of the lipid nanoparticles and mRNAs comprising a wild-type ORF encoding the polypeptide.

46. The lipid nanoparticle of claim 45, wherein storage of the lipid nanoparticle is conducted at a temperature between about 2° C. to about 8° C.

47. The lipid nanoparticle of any one of claims 42-46, further comprising a stabilizing compound of Formula (I): 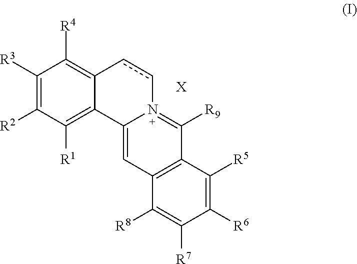
or a tautomer or solvate thereof, wherein: [symbol]
is a single bond or a double bond;
R¹ is H;
R² is OCH₃, or together with R³ is OCH₂O;
R³ is OCH₃, or together with R² is OCH₂O;
R⁴ is H;
R⁵ is H or OCH₃;
R⁶ is OCH₃;
R⁷ is H or OCH₃;
R⁸ is H;
R⁹ is H or CH₃; and
X is a pharmaceutically acceptable anion.

48. The lipid nanoparticle of claim 47, wherein the stabilizing compound is wherein the compound is of: 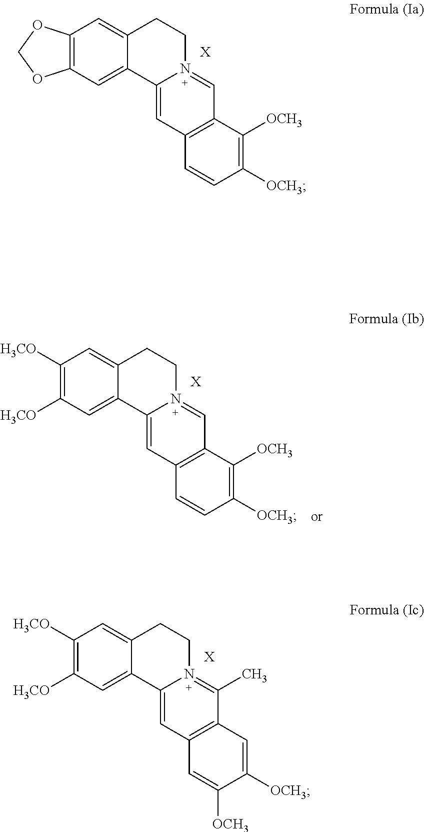
or a tautomer or solvate thereof.

49. The lipid nanoparticle of any one of claims 42-48, further comprising a stabilizing compound of Formula (II): 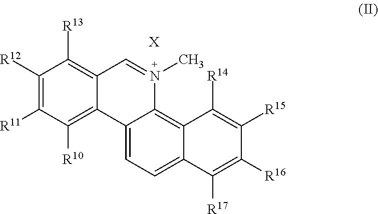
or a tautomer or solvate thereof, wherein:
R¹⁰ is H;
R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion.

50. A pharmaceutical composition comprising the lipid nanoparticle of any one of claims 42-49, and a pharmaceutically acceptable excipient.

51. A method of producing a modified mRNA sequence comprising an ORF encoding a polypeptide, the method comprising modifying a reference mRNA sequence comprising a reference ORF to produce the modified mRNA sequence by:
(a) replacing one or more codons in the reference ORF comprising a CpA dinucleotide with a codon that encodes the same amino acid but does not comprise a CpA dinucleotide; and/or
(b) replacing one or more codons in the reference ORF that:
(1) ends in a cytidine nucleotide; and
(2) is immediately followed in the reference ORF by a codon that encodes an isoleucine, methionine, threonine, asparagine, or lysine, or a codon that encodes a serine or arginine and begins with an adenosine nucleotide.
with a codon encoding the same amino acid as the replaced codon but does not end in a cytidine nucleotide.

52. The method of claim 51, wherein the reference mRNA sequence further comprises:
(i) a reference 5′ untranslated region (UTR); and/or
(ii) a reference 3′ UTR.

53. The method of claim 52, wherein the reference 5′ UTR is a heterologous 5′ UTR and/or the reference 3′ UTR is a heterologous 3′ UTR.

54. The method of claim 52 or 53, wherein the replacing comprises changing the last nucleotide of the reference 5′ UTR from a cytidine nucleotide to a non-cytidine nucleotide.

55. The method of any one of claims 52-54, wherein the reference mRNA sequence further comprises:
(iii) a 5′ cap structure; and/or
(iv) a poly-A region.

56. The method of claim 55, wherein the replacing comprises changing the last nucleotide of the reference 3′ UTR from a cytidine nucleotide to a non-cytidine nucleotide.

57. The method of any one of claims 51-56, further comprising replacing one or more cytidine nucleotides in the reference mRNA sequence with guanosine nucleotides.

58. The method of any one of claims 51-57, further comprising replacing one or more unmodified cytidine nucleotides in the reference mRNA sequence with modified cytidine nucleotides.

59. The method of any one of claims 51-58, further comprising replacing one or more unmodified adenosine nucleotides in the reference mRNA sequence with modified adenosine nucleotides.

60. The method of any one of claims 51-59, further comprising replacing one or more adenosine nucleotides in the reference mRNA sequence with uracil nucleotides.

61. The method of any one of claims 51-60, further comprising replacing one or more adenosine nucleotides in the reference mRNA sequence, that are not immediately followed by a second adenosine nucleotide, with cytidine nucleotides.

62. The method of any one of claims 51-61, further comprising replacing one or more adenosine nucleotides in the reference mRNA sequence with guanosine nucleotides.

63. The method of any one of claims 51-62, wherein the ORF of the modified mRNA sequence comprises a number of CpA dinucleotides that is greater than or equal to the theoretical minimum and less than or equal to 300% of the theoretical minimum.

64. The method of any one of claims 51-63, wherein the ORF of the modified mRNA sequences comprises a number of CpA dinucleotides that is:
(i) greater than or equal to a theoretical minimum; and
(ii) no more than 11 CpA dinucleotides per 100 nucleotides of the ORF greater than the theoretical minimum.

65. The method of claim 64, wherein the number of CpA dinucleotides per 100 nucleotides of the ORF greater than the theoretical minimum is no more than 10, no more than 9, no more than 8, no more than 7, no more than 6, no more than 5, no more than 4, no more than 3, no more than 2, or no more than 1.

66. The method of any one of claims 51-65, wherein the ORF of the modified mRNA sequence comprises a CpA dinucleotide content of 6.5% or less.

67. The method of claim 66, wherein the ORF of the modified mRNA sequence comprises a CpA dinucleotide content of 6.0% or less, 5.5% or less, 5% or less, 4.5% or less, 4% or less, 3.5% or less, 3.0% or less, 2.5% or less, 2.0% or less, 1.5% or less, 1.0% or less, or 0.5% or less.

68. The method of any one of claims 51-67, wherein, in the modified mRNA sequence:
(a) fewer than 30% of amino acids that immediately precede an isoleucine residue in the polypeptide are encoded by codons in the ORF that end in cytidine nucleotides;
(b) fewer than 30% of amino acids that immediately precede a methionine residue in the polypeptide are encoded by codons in the ORF that end in cytidine nucleotides;
(c) fewer than 30% of amino acids that immediately precede a threonine residue in the polypeptide are encoded by codons in the ORF that end in cytidine nucleotides;
(d) fewer than 30% of amino acids that immediately precede an asparagine residue in the polypeptide are encoded by codons in the ORF that end in cytidine nucleotides;
(e) fewer than 30% of amino acids that immediately precede a lysine residue in the polypeptide are encoded by codons in the ORF that end in cytidine nucleotides;
(f) fewer than 30% of amino acids that immediately precede a serine residue, wherein the serine residue is encoded by a codon in the ORF beginning with an adenosine nucleotide, are encoded by codons in the ORF that end in cytidine nucleotides; and/or
(g) fewer than 30% of amino acids that immediately precede an arginine residue, wherein the arginine residue is encoded by a codon in the ORF beginning with an adenosine nucleotide, are encoded by codons in the ORF that end in cytidine nucleotides.

69. The method of any one of claims 51-68, wherein, in the modified mRNA sequence, fewer than 15% of serine residues, fewer than 27% of proline residues, fewer than 28% of threonine residues, and fewer than 23% of alanine residues in the polypeptide are encoded by codons in the ORF that comprise a CpA dinucleotide.

70. The method of any one of claims 51-69, wherein, in the modified mRNA sequence:
(a) no serine residue of the polypeptide is encoded by a codon in the ORF comprising a CpA dinucleotide;
(b) no proline residue of the polypeptide is encoded by a codon in the ORF comprising a CpA dinucleotide;
(c) no threonine residue of the polypeptide is encoded by a codon in the ORF comprising a CpA dinucleotide; and/or
(d) no alanine residue of the polypeptide is encoded by a codon in the ORF comprising a CpA dinucleotide.

71. The method of any one of claims 51-70, wherein, in the modified mRNA sequence:
(a) no amino acid that immediately precedes an isoleucine residue in the polypeptide is encoded by a codon in the ORF that ends in a cytidine nucleotide;
(b) no amino acid that immediately precedes a methionine residue in the polypeptide is encoded by a codon in the ORF that ends in a cytidine nucleotide;
(c) no amino acid that immediately precedes a threonine residue in the polypeptide is encoded by a codon in the ORF that ends in a cytidine nucleotide;
(d) no amino acid that immediately precedes an asparagine residue in the polypeptide is encoded by a codon in the ORF that ends in a cytidine nucleotide;
(e) no amino acid that immediately precedes a lysine residue in the polypeptide is encoded by a codon in the ORF that ends in a cytidine nucleotide;
(f) no amino acid that immediately precedes a serine residue, wherein the serine residue is encoded by a codon in the ORF beginning with an adenosine nucleotide, is encoded by a codon in the ORF that ends in a cytidine nucleotide; and/or
(g) no amino acid that immediately precedes an arginine residue, wherein the arginine residue is encoded by a codon in the ORF beginning with an adenosine nucleotide, is encoded by a codon in the ORF that ends in a cytidine nucleotide.

72. The method of any one of claims 51-71, wherein, in the modified mRNA sequence, no amino acid that immediately precedes an isoleucine, methionine, threonine, asparagine, lysine residue in the polypeptide is encoded by a codon that ends in a cytidine nucleotide.

73. The method of any one of claims 51-72, wherein, in the modified mRNA sequence, no codon in the ORF beginning with an adenosine nucleotide is immediately preceded by a codon in the ORF that ends in a cytidine nucleotide.

74. The method of any one of claims 51-73, wherein the modified mRNA sequence comprises a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%.

75. The method of any one of claims 51-74, wherein one or more nucleotides of the modified mRNA sequence comprises a chemically modified nucleotide.

76. The method of any one of claims 51-74, wherein each of the uridine nucleotides of the modified mRNA sequence comprises a chemically modified nucleotide.

77. The method of claim 75 or 76, wherein the chemically modified nucleotide comprises N₁-methylpseudouridine.

78. The method of any one of claims 75-77, wherein one or more CpA dinucleotides of the modified mRNA sequence comprises a modified cytidine nucleotide and/or a modified adenosine nucleotide.

79. The method of any one of claims 51-78, wherein the number of CpA dinucleotides comprising an unmodified cytidine nucleotide and an unmodified adenosine nucleotide in the ORF of the modified mRNA sequence is 100%, 95% or less, 90% or less, 80% or less, 70% or less, 60% or less, 50% or less, 40% or less, 30% or less, 20% or less, or 10% or less of the total number of histidine and glutamine residues in the polypeptide.

80. The method of any one of claims 51-79, wherein the polypeptide comprises 9-5,000, 20-4,000, 30-3,000, 40-2,000, or 50-1,500 amino acids.

81. The mRNA of any one of claims 51-80, wherein the polypeptide is a vaccine antigen or a therapeutic protein.

82. The method of any one of claims 51-81, wherein the ORF of the modified mRNA sequence is codon-optimized for expression in a cell.

83. The method of claim 82, wherein the cell is a mammalian cell.

84. The method of claim 82 or 83, wherein the cell is a human cell.

85. The method of any one of claims 51-84, further comprising transcribing the modified mRNA sequence to produce a modified mRNA.

86. The method of claim 85, wherein a level of expression in a mammalian cell of the encoded polypeptide from the modified mRNA is at least 80% of a level of expression of the reference mRNA.

87. The method of claim 85 or 86, wherein a coefficient of degradation at 25° C. of the modified mRNA is 90% or less, 80% or less, 70% or less, 60% or less, or 50% or less, relative to an mRNA comprising the reference ORF.

88. The method of any one of claims 85-87, wherein a composition comprising a plurality of the mRNAs is remains at least above 50% purity for at least 30 days, at least 60 days, at least 90 days, at least 120 days, at least 150 days, or at least 180 days longer in storage than a composition comprising a plurality of mRNAs comprising the reference ORF.

89. The method of claim 88, wherein storage of the modified mRNA is conducted at a temperature between about 2° C. to about 8° C.

90. The method of any one of claims 85-89, wherein the modified mRNA has increased stability relative to a reference mRNA comprising the reference mRNA sequence.
